SEQUENCE LISTING (56 sequences)

SEQ ID NO: 1 - DNA, 1560 bp, Drosophila melanogaster
atgctgaggaacgaatacataagcaataacaagaaggtgctcaactcggaccaaaacaagtactacatgctaacggtcgaggaggccgatatccaaggcacgaacatgtccgacggcgtcagcatcttgcacatcaaacaggaggtggacactccatcggcgtcctgctttagtcccagctccaagtcaacggccacgcagagtggcacaaacggcctgaaatcctcgccctcggtttcgccggaaaggcagctctgcagctcgacgacctctctatcctgcgatttgcacaatgtatccttaagcaatgatggcgatagtctgaaaggaagtggtacaagtggcggcaatggcggaggaggaggtggtggtacgagtggtggaaatgcgaccaatgcgagtgccggagctggatcgggatccgtcagggacgagctccgccgattgtgtttggtttgtggcgatgtggccagtggattccactatggtgtggcgagttgtgaggcttgcaaagcgttctttaaacgcaccatccaaggcaacatcgagtacacgtgtccggcgaacaacgagtgtgagattaacaagcggagacgcaaggcctgccaagcgtgtcgcttccagaaatgtctactaatgggcatgctcaaggagggtgtgcgcttggatcgagttcgtggaggacggcagaagtaccgaaggaatcctgtatcaaactcttaccagactatgcagctgctataccaatccaacaccacctcgctgtgcgatgtcaagatactggaggtgctcaattcatatgagccggatgccttgagcgtccaaacgccgccgccgcaagtccacacgactagcataactaatgatgaggcctcatcctcctcgggcagcataaaactggagtccagcgttgttacgcccaatgggacttgcattttccaaaacaacaacaacaatgatcccaatgagatactaagcgtccttagtgatatttacgacaaggaattggtcagcgtcattggctgggccaagcagatacctggctttatagatctgccacttaacgaccagatgaagcttctccaggtgtcgtgggcagagatcctgacgctccagctgaccttccggtccctaccgttcaatggcaagttatgcttcgccacggatgtctggatggatgaacatttggccaaggagtgcggttacacggagttctactaccactgcgtccagatcgcacagcgcatggaaagaatatcgccacgaagggaggagtactacttgctaaaggcgctcctgctggccaactgcgacattctgctggatgatcagagttccctgcgcgcatttcgtgatacgattcttaattctctaaacgatgtggtctacttgctgcgtcattcgtcggccgtgtcgcatcagcaacaattgctgcttttgctgccttcgctgcggcaggcggatgatatcctgcgaagattttggcgtggaattgcacgcgatgaagtcattaccatgaagaaactgttcctcgagatgctcgagccgctggccaggtga

SEQ ID NO: 2 - protein, 519 aa, Drosophila melanogaster
MLRNEYISNNKKVLNSDQNKYYMLTVEEADIQGTNMSDGVSILHIKQEVDTPSASCFSPSSKSTATQSGTNGLKSSPSVSPERQLCSSTTSLSCDLHNVSLSNDGDSLKGSGTSGGNGGGGGGGTSGGNATNASAGAGSGSVRDELRRLCLVCGDVASGFHYGVASCEACKAFFKRTIQGNIEYTCPANNECEINKRRRKACQACRFQKCLLMGMLKEGVRLDRVRGGRQKYRRNPVSNSYQTMQLLYQSNTTSLCDVKILEVLNSYEPDALSVQTPPPQVHTTSITNDEASSSSGSIKLESSVVTPNGTCIFQNNNNNDPNEILSVLSDIYDKELVSVIGWAKQIPGFIDLPLNDQMKLLQVSWAEILTLQLTFRSLPFNGKLCFATDVWMDEHLAKECGYTEFYYHCVQIAQRMERISPRREEYYLLKALLLANCDILLDDQSSLRAFRDTILNSLNDVVYLLRHSSAVSHQQQLLLLLPSLRQADDILRRFWRGIARDEVITMKKLFLEMLEPLAR

SEQ ID NO: 3 - DNA, 1455 bp, Drosophila melanogaster
atgtccgacggcgtcagcatcttgcacatcaaacaggaggtggacactccatcggcgtcctgctttagtcccagctccaagtcaacggccacgcagagtggcacaaacggcctgaaatcctcgccctcggtttcgccggaaaggcagctctgcagctcgacgacctctctatcctgcgatttgcacaatgtatccttaagcaatgatggcgatagtctgaaaggaagtggtacaagtggcggcaatggcggaggaggaggtggtggtacgagtggtggaaatgcgaccaatgcgagtgccggagctggatcgggatccgtcagggacgagctccgccgattgtgtttggtttgtggcgatgtggccagtggattccactatggtgtggcgagttgtgaggcttgcaaagcgttctttaaacgcaccatccaaggcaacatcgagtacacgtgtccggcgaacaacgagtgtgagattaacaagcggagacgcaaggcctgccaagcgtgtcgcttccagaaatgtctactaatgggcatgctcaaggagggtgtgcgcttggatcgagttcgtggaggacggcagaagtaccgaaggaatcctgtatcaaactcttaccagactatgcagctgctataccaatccaacaccacctcgctgtgcgatgtcaagatactggaggtgctcaattcatatgagccggatgccttgagcgtccaaacgccgccgccgcaagtccacacgactagcataactaatgatgaggcctcatcctcctcgggcagcataaaactggagtccagcgttgttacgcccaatgggacttgcattttccaaaacaacaacaacaatgatcccaatgagatactaagcgtccttagtgatatttacgacaaggaattggtcagcgtcattggctgggccaagcagatacctggctttatagatctgccacttaacgaccagatgaagcttctccaggtgtcgtgggcagagatcctgacgctccagctgaccttccggtccctaccgttcaatggcaagttatgcttcgccacggatgtctggatggatgaacatttggccaaggagtgcggttacacggagttctactaccactgcgtccagatcgcacagcgcatggaaagaatatcgccacgaagggaggagtactacttgctaaaggcgctcctgctggccaactgcgacattctgctggatgatcagagttccctgcgcgcatttcgtgatacgattcttaattctctaaacgatgtggtctacttgctgcgtcattcgtcggccgtgtcgcatcagcaacaattgctgcttttgctgccttcgctgcggcaggcggatgatatcctgcgaagattttggcgtggaattgcacgcgatgaagtcattaccatgaagaaactgttcctcgagatgctcgagccgctggccaggtga

SEQ ID NO: 4 - protein, 484 aa, Drosophila melanogaster
MSDGVSILHIKQEVDTPSASCFSPSSKSTATQSGTNGLKSSPSVSPERQLCSSTTSLSCDLHNVSLSNDGDSLKGSGTSGGNGGGGGGGTSGGNATNASAGAGSGSVRDELRRLCLVCGDVASGFHYGVASCEACKAFFKRTIQGNIEYTCPANNECEINKRRRKACQACRFQKCLLMGMLKEGVRLDRVRGGRQKYRRNPVSNSYQTMQLLYQSNTTSLCDVKILEVLNSYEPDALSVQTPPPQVHTTSITNDEASSSSGSIKLESSVVTPNGTCIFQNNNNNDPNEILSVLSDIYDKELVSVIGWAKQIPGFIDLPLNDQMKLLQVSWAEILTLQLTFRSLPFNGKLCFATDVWMDEHLAKECGYTEFYYHCVQIAQRMERISPRREEYYLLKALLLANCDILLDDQSSLRAFRDTILNSLNDVVYLLRHSSAVSHQQQLLLLLPSLRQADDILRRFWRGIARDEVITMKKLFLEMLEPLAR

SEQ ID NO: 5 - DNA, 2106 bp, Drosophila melanogaster
atgaccactgtcaaggcggagaagccggtaaccaccacttgctcctactgggtgttacgcaaaaagcgccgctgcaagatgactgcaaacaagggcagtgagttttgtggagcccatgcctcatcggcagccaccactgcaacttgcgatgaaaaagctagagaagattccttccaggaacgtattccctgcccgcttgatcacaaacataccgtgtttaaaagaaaactagccaaacacttgaccatttgcaatgccagggatcaggaaagttcactgccctacattgtaaaaggagtgaattctggagacaatcttaaggaaacagacgaagatttgaacaaattcaatcaaataaaactgcacgaactggcggatgaagagttctacagtctgatcgacaaagttaagaatctgtatgacaagcacatcaattccagcatacaggaactgcagctggagcacgaatccctaaaggaggaacttagtcgcaaggactacggccaggaaacgctacgccagctcacccagaccactagcttgctgggcatcctggaacacgatcaccagttgatggatcacacgagctatatagaattcggagccggaaagggacaattggcctactttttggccaccgtattgcaggaacagaagttgagtcactcacaggtggtcctcatcgatcgaatgtccctgcggcacaagaaggacaacaagttggccaacagggaggtggtgcaacgtatccgtgctgatatcgctgatcttaagctttctgctctgccggagctaaagaaaacccaacgaacggtggctttttcgaagcacctctgtggagcagctacagacttgaccctgagatgtatactcggtgatggaaatgccagctcggactacgttctcatcgccctgtgttgtcatcatcgctgctcctggcgctcctacgtgggacgcaagtttcttcaggaagctggaattggaccacgggagttcgttatcctaaccaaaatggtcagttgggcagtttgtggcacgggcttaagccgagagcggcgtaaggccatggaatcggctgactttcagctcaccgagacgaacacgcagcgtctaactcgccaggagcgcgaacaaattggccaacagtgcaagcgggttctggattatggacgactggagcatctacgatcccacggatatcaagcagaacttaagttctatgttcccagggatgtgactttggaaaaccacatccaggtggcatataaaaccgccaatatccgggatgagctgattcagtccgcggcgactctttggctcattggcaagcagtgccaaccggtgaccatgtcgaacttcagtgcctgcgcagtgtgcggcgatcagagctccgggaagcactacggcgtgtcctgctgcgatgggtgctcctgctttttcaagcggagcgtgcggcgcgggagcagctacgcctgcatcgctctggtcgggaactgtgtggtggacaaggcgcggcggaactggtgtccctcctgccgcttccagcgatgcctggccgtgggaatgaacgctgctgcggttcaggaggagcgcggtccgcgcaaccagcaggtggctctctaccgcactggccggagacaagctccgccatctcaggcggcgccatccccgacgccccactcccaggcgctgcacttccagatcctcgcccagatccttgtcacgtgcctgcgccaggcgaaggccaacgagcagttcgctctgttggatcgctgccaacaagacgccatctttcaggtggtgtggagcgagatcttcgtcctgcgagcgtcccactggtctctggacatcagcgccatgatcgacggctgcggcgatgagcagctcaaacggctcatttgcgaggcccaccagctaagggccgacgtcctggaactcaactttatggagtccctaatcctgtgcagaaaagaattggccatcaatgcggagtatgccgttatcctgggaagccactctaaagccgccctgatctccttagcccgctacaccctgcagcaatccaactacctgcgggtggtcagggacatcttaaaaacactttag

SEQ ID NO: 6 - protein, 701 aa, Drosophila melanogaster
MTTVKAEKPVTTTCSYWVLRKKRRCKMTANKGSEFCGAHASSAATTATCDEKAREDSFQERIPCPLDHKHTVFKRKLAKHLTICNARDQESSLPYIVKGVNSGDNLKETDEDLNKFNQIKLHELADEEFYSLIDKVKNLYDKHINSSIQELQLEHESLKEELSRKDYGQETLRQLTQTTSLLGILEHDHQLMDHTSYIEFGAGKGQLAYFLATVLQEQKLSHSQVVLIDRMSLRHKKDNKLANREVVQRIRADIADLKLSALPELKKTQRTVAFSKHLCGAATDLTLRCILGDGNASSDYVLIALCCHHRCSWRSYVGRKFLQEAGIGPREFVILTKMVSWAVCGTGLSRERRKAMESADFQLTETNTQRLTRQEREQIGQQCKRVLDYGRLEHLRSHGYQAELKFYVPRDVTLENHIQVAYKTANIRDELIQSAATLWLIGKQCQPVTMSNFSACAVCGDQSSGKHYGVSCCDGCSCFFKRSVRRGSSYACIALVGNCVVDKARRNWCPSCRFQRCLAVGMNAAAVQEERGPRNQQVALYRTGRRQAPPSQAAPSPTPHSQALHFQILAQILVTCLRQAKANEQFALLDRCQQDAIFQVVWSEIFVLRASHWSLDISAMIDGCGDEQLKRLICEAHQLRADVLELNFMESLILCRKELAINAEYAVILGSHSKAALISLARYTLQQSNYLRVVRDILKTL

SEQ ID NO: 7 - DNA, 837 bp, Drosophila melanogaster
atgtcgaacttcagtgcctgcgcagtgtgcggcgatcagagctccgggaagcactacggcgtgtcctgctgcgatgggtgctcctgctttttcaagcggagcgtgcggcgcgggagcagctacgcctgcatcgctctggtcgggaactgtgtggtggacaaggcgcggcggaactggtgtccctcctgccgcttccagcgatgcctggccgtgggaatgaacgctgctgcggttcaggaggagcgcggtccgcgcaaccagcaggtggctctctaccgcactggccggagacaagctccgccatctcaggcggcgccatccccgacgccccactcccaggcgctgcacttccagatcctcgcccagatccttgtcacgtgcctgcgccaggcgaaggccaacgagcagttcgctctgttggatcgctgccaacaagacgccatctttcaggtggtgtggagcgagatcttcgtcctgcgagcgtcccactggtctctggacatcagcgccatgatcgacggctgcggcgatgagcagctcaaacggctcatttgcgaggcccaccagctaagggccgacgtcctggaactcaactttatggagtccctaatcctgtgcagaaaagaattggccatcaatgcggagtatgccgttatcctgggaagccactctaaagccgccctgatctccttagcccgctacaccctgcagcaatccaactacctgcggttcggacaactgctccttggtctgaggcagctgtgcctgaggcgcttcgactgcgcgctttcttgtatgtttcgcagcgtggtcagggacatcttaaaaacactttaa

SEQ ID NO: 8 - protein, 278 aa, Drosophila melanogaster
MSNFSACAVCGDQSSGKHYGVSCCDGCSCFFKRSVRRGSSYACIALVGNCVVDKARRNWCPSCRFQRCLAVGMNAAAVQEERGPRNQQVALYRTGRRQAPPSQAAPSPTPHSQALHFQILAQILVTCLRQAKANEQFALLDRCQQDAIFQVVWSEIFVLRASHWSLDISAMIDGCGDEQLKRLICEAHQLRADVLELNFMESLILCRKELAINAEYAVILGSHSKAALISLARYTLQQSNYLRFGQLLLGLRQLCLRRFDCALSCMFRSVVRDILKTL

SEQ ID NO: 9 - DNA, 1626 bp, Drosophila melanogaster
atgcacggacaggcgcctccacctacatcaacgggcgtggccccgcccacacagccaccgccccctcatcccgccgccccaaacgtgcccaatggtcgattgctgagctggaatcacagtgccgctgcagctgctgcggcggcggcagcccaagcggcagccaactccatgaaccactcgtcggcggcggagggttcatcgatgacccggattaagggtcagaacctgggcctcatctgcgtggtgtgcggcgacaccagctcgggaaagcactacggaatcctagcctgcaatggctgctccggattcttcaaacgcagcgcgggaacgggacgctgtgtggtggacaaagctcatcggaatcaatgccaggcctgcaggctcaagaagtgccttcaaatgggaatgaacaaggacgccgtgcaaaatgagcgacagccccgcaacacggccactatacggccggagactctgcgggagatggagcacggacgggcgctgcgagaggccgccgtggccgtcggggttttcgggccacccgtgctactgtctccgccctgctacggctccggactcctgccgccgccctcactgggcagtttgccgacgggacgattgctgcaccacaaccacctcacctcctcaatgcaattggctgcgaaccacatgggcgccggcagctttcccatgttcaacgcagccggcgtgcaccactcgcccaaggaaaaggcctacggcatggagatggccaccagtggcaatgtctcccactcgaccaactcgagcagcaaccactccatcgacccgagctcaccggcgccggaaaacgccaaggagataaacatcgccggcggcagtgtctcctccgtcagctcttccagtcccaccatggaaaatgacaatgatgacgactccatagatgtaaccaacgacaacgaggagccgcatgcagtcagcagatcggattcgagtttcattatgccgcagttcatgtcgcccaatctgtacacccatcaacacgaaacagtttacgagacaagtgcccggctgctcttcatggccgtcaagtgggccaagaacctgcccagctttgcaagactttcctttcgggatcaggtgattttgctggaggagtcctggtcggagctgttcctgctgaacgcaatccaatggtgcattcccctggatcccaccggctgcgccctcttctcggtggcggagcactgcaataatctagagaacaatgccaatggcgacacttgcataacaaaggaggagctggcggcggatgtgcgaacgctccacgagatcttctgcaaatacaaggcggtgctggtggaccccgctgaattcgcgtgcctcaaggcgatagttctcttccggccggaaacgcgcggacttaaagatccggcgcagatagagaatcttcaggatcaggcgcacgtaatgctgtcgcagcacacaaagacgcagttcaccgcccagatagccagattcggacgactccttctcatgctgccgttgctgcgcatgatcagctcccacaagattgagtccatctattttcagcgcactattgggaacacgcccatggaaaaggtgctctgtgacatgtataagaactag

SEQ ID NO: 10 - protein, 541 aa, Drosophila melanogaster
MHGQAPPPTSTGVAPPTQPPPPHPAAPNVPNGRLLSWNHSAAAAAAAAAAQAAANSMNHSSAAEGSSMTRIKGQNLGLICVVCGDTSSGKHYGILACNGCSGFFKRSAGTGRCVVDKAHRNQCQACRLKKCLQMGMNKDAVQNERQPRNTATIRPETLREMEHGRALREAAVAVGVFGPPVLLSPPCYGSGLLPPPSLGSLPTGRLLHHNHLTSSMQLAANHMGAGSFPMFNAAGVHHSPKEKAYGMEMATSGNVSHSTNSSSNHSIDPSSPAPENAKEINIAGGSVSSVSSSSPTMENDNDDDSIDVTNDNEEPHAVSRSDSSFIMPQFMSPNLYTHQHETVYETSARLLFMAVKWAKNLPSFARLSFRDQVILLEESWSELFLLNAIQWCIPLDPTGCALFSVAEHCNNLENNANGDTCITKEELAADVRTLHEIFCKYKAVLVDPAEFACLKAIVLFRPETRGLKDPAQIENLQDQAHVMLSQHTKTQFTAQIARFGRLLLMLPLLRMISSHKIESIYFQRTIGNTPMEKVLCDMYKN

SEQ ID NO: 11 - DNA, 1599 bp, Drosophila melanogaster
atggcgaccgggcgttctctgctctttcgagtgccttggtatgtgtgcttgtgtgtgtgcgcagagagcgcagagccgggtgtttattggagattgcgattgcggcttggcttacccacactcgcagggccgcacaccaacacactaacactaacagcgaggacaagctcctgccgcagcatcaagaaggaacgaatcaaagcaagccaacaagcaaatgcgccaccagagttgccactaaaagtctccgttgacgttaacatcatcatcgcggcacactcgcagcgccgtcggatcggattggttcggtttcatcagcgggaatcagaggaccgtccacttgccgtcgcctctccacgattgcaaattaatatggagcctactgcgatgaacccgaaaaaactccacagtccgcagcggcattgctacactccgccgccggcgccgatgcacggacaggcgcctccacctacatcaacgggcgtggccccgcccacacagccaccgccccctcatcccgccgccccaaacgtgcccaatggtcgattgctgagctggaatcacagtgccgctgcagctgctgcggcggcggcagcccaagcggcagccaactccatgaaccactcgtcggcggcggagggttcatcgatgacccggattaagggtcagaacctgggcctcatctgcgtggtgtgcggcgacaccagctcgggaaagcactacggaatcctagcctgcaatggctgctccggattcttcaaacgcagcgtgcggcggaaactcatttatcgctgccaggcgggaacgggacgctgtgtggtggacaaagctcatcggaatcaatgccaggcctgcaggctcaagaagtgccttcaaatgggaatgaacaaggacgacgactccatagatgtaaccaacgacaacgaggagccgcatgcagtcagcagatcggattcgagtttcattatgccgcagttcatgtcgcccaatctgtacacccatcaacacgaaacagtttacgagacaagtgcccggctgctcttcatggccgtcaagtgggccaagaacctgcccagctttgcaagactttcctttcgggatcaggtaattttgctggaggagtcctggtcggagctgttcctgctgaacgcaatccaatggtgcattcccctggatcccaccggctgcgccctcttctcggtggcggagcactgcaataatctagagaacaatgccaatggcgacacttgcataacaaaggaggagctggcggcggatgtgcgaacgctccacgagatcttctgcaaatacaaggcggtgctggtggaccccgctgaattcgcgtgcctcaaggcgatagttctcttccggccggaaacgcgcggacttaaagatccggcgcagatagagaatcttcaggatcaggcgcaccacacaaagacgcagttcaccgcccagatagccagattcggacgactccttctcatgctgccgttgctgcgcatgatcagctcccacaagattgagtccatctattttcagcgcactattgggaacacgcccatggaaaaggtgctctgtgacatgtataagaactag

SEQ ID NO: 12 - protein, 532 aa, Drosophila melanogaster
MATGRSLLFRVPWYVCLCVCAESAEPGVYWRLRLRLGLPTLAGPHTNTLTLTARTSSCRSIKKERIKASQQANAPPELPLKVSVDVNIIIAAHSQRRRIGLVRFHQRESEDRPLAVASPRLQINMEPTAMNPKKLHSPQRHCYTPPPAPMHGQAPPPTSTGVAPPTQPPPPHPAAPNVPNGRLLSWNHSAAAAAAAAAAQAAANSMNHSSAAEGSSMTRIKGQNLGLICVVCGDTSSGKHYGILACNGCSGFFKRSVRRKLIYRCQAGTGRCVVDKAHRNQCQACRLKKCLQMGMNKDDDSIDVTNDNEEPHAVSRSDSSFIMPQFMSPNLYTHQHETVYETSARLLFMAVKWAKNLPSFARLSFRDQVILLEESWSELFLLNAIQWCIPLDPTGCALFSVAEHCNNLENNANGDTCITKEELAADVRTLHEIFCKYKAVLVDPAEFACLKAIVLFRPETRGLKDPAQIENLQDQAHHTKTQFTAQIARFGRLLLMLPLLRMISSHKIESIYFQRTIGNTPMEKVLCDMYKN

SEQ ID NO: 13 - DNA, 4563 bp, Drosophila melanogaster
atgacactgagccgtggcccgtacagcgagctcgataaaatgagcctttttcaagacctcaaactcaaacggcgcaaaatcgattcgcgatgcagcagtgacggcgagtccatagcggacacgtccacctcgtcgccggacctgctggcgcccatgtcgccgaagctctgcgacagcggctcggcgggggcgtcgctgggggcatcgctgcccctgccgctggccctgcccctgccaatggccctgccactgcccatgtcgctgcccctgcccctcacggcggcatcttcggcggtcaccgtttcgctggcagcggtcgtggccgcggtggccgagacgggtggcgcgggcgcgggaggagctgggacagcagtaacagcgtcgggagcaggaccatgcgtctccacgtcgtctacgacggcagcggcagccacatcctcgacctcctcgctctcgtcctcctcctcttcgtcatcctccacgtcctccagcacttcctccgcctcgccgacagctggagcctcctccacggccacctgccccgccagcagcagcagcagcagtggaaacggaagtgggggcaaaagtggtagcatcaagcaggagcacacggagatacactcgtcgagcagtgcgatttcggcggccgccgcctcaacggtgatgtcaccgccgcccgctgaggcgacgagatccagtccagccacgcccgagggaggcggaccagctggcgacggaagtggagcaacgggaggcggaaacacgagcggcggatcaacggctggagtggccattaatgaacaccaaaacaatggcaatggcagcggcgggagcagtcgagcctctcccgattcgctggaagagaagccctctaccacaacgaccacaggtcgtccaacgctcacgcccacgaatggggtgctgtcctccgcctcggcgggcacggggatttccacaggaagcagcgccaagctgagcgaggctggtatgagtgtgatacggtccgtgaaggaggagcgcttgctcaacgtatccagcaagatgctggtgttccatcagcagcgggagcaagagaccaaagcagtggcggctgcagcagcagcagcagcggcgggccatgtgacggttctagtgacgccatcgcgcatcaaatcggagccaccgccgccggcttcaccctcctctacatccagcacacaaagggaaagggaacgggaacgcgatcgagagagggatcgcgaaagggaacgcgagcgggaccgggaccgggaacgggaacgggaacagtccatcagctcctcgcagcagcacctaagtcgggtctccgccagtccacccactcagctgtcccacggcagcctgggacccaacattgtgcagacgcaccatcttcaccagcaactcacacagccgctgacgctgcgcaagagcagcccgcccacagagcacctgctcagtcagtccatgcaacatctcacacagcagcaggcgatccacctgcatcacctacttggccagcagcagcagcagcagcaggcgtcgcatccccagcagcaacagcagcagcaacactcgccccactccctggtgcgggtgaaaaaggaaccgaatgttggtcagcggcacttatcgccgcatcaccaacaacagtcgccactcctgcagcaccaccaacagcagcagcagcagcaacaacaacagcaacagcatctgcatcagcaacagcaacagcagcagcatcaccagcagcagccccaggcactggccctgatgcatccggcttccctggcgctaaggaacagcaatcgggatgcggccattctgtttcgggtgaagagcgaagtgcaccagcaggtggccgccgggctgccgcatctgatgcagtccgctggtggggcagcggccgccgccgcagcagctgtggccgctcagcgaatggtatgcttcagcaatgccaggatcaatggcgttaagccggaggtgattggaggaccgctgggcaacctgcggcccgtgggcgtcggtggcggaaacggaagtggctccgtgcagtgcccctcgccgcatccatcctcctcgtcgtcatcctcgcagctgtcgccgcagacgccctcccagacgccgccccgaggcacgcccaccgtcataatgggcgagagctgcggggtgcgcaccatggtctggggctacgagcctccgccaccctcggcgggccagtcccacggccagcacccgcaacagcaacagcagtcgccccaccaccagccgcaacaacaacagcagcagcaacaacagcagtcgcagcagcaacagcaacagcagcagcaacagtcgctgggccagcagcagcactgcctctcctcgccgtcggcgggatcgctgacgccctcctcttcgtccggcggtggttcggtatctggcggcggagtgggcggaccactcacaccctcctcggtggcgccgcagaataacgaggaggccgcccaactcctgcttctccctgggacagacacgcatccaggacatgagatcacgggccacacccccttccgcacaccgcacgcccttaatatggagcggctgtgggcgggagactactcgcaattgccgcccggccagctgcaggctctgaatctcagtgcccaacagcagcagtggggcagcagcaactccacgggtcttggtggcgtaggcggcggcatgggcggacgcaacctggaggcgccgcacgagccgaccgacgaggacgaacagccgctcgtttgcatgatctgcgaggacaaggccaccggcctgcactacggcatcatcacctgcgaggggtgcaagggcttcttcaagcggacggtgcagaaccgacgagtctacacctgcgtggcggacggcacctgcgagataaccaaagcacagcgcaaccgttgtcagtattgtcgatttaagaagtgcatcgagcagggcatggtgctgcaagccgttcgcgaggatcgcatgccgggcggtcgcaacagtggcgccgtctacaatttgtacaaggtgaagtacaagaagcacaagaagaccaatcagaagcagcagcagcaggccgcccagcagcagcagcagcaggcggcggcgcagcagcagcaccagcaacagcagcagcatcaacagcaccagcaacatcagcaacagcagttgcactcgccgctccaccatcaccaccaccagggccaccagtcgcaccacgcgcagcagcagcaccacccacagctgtcgccgcaccacctgctgtcgccgcagcagcagcaacttgccgccgcggtggcagcagctgcgcagcaccaacagcaacagcaacaacagcagcaacagcagcagcaggccaagctgatgggcggcgtggtggacatgaagcccatgttcctcggccccgctttgaagccggagttgctgcaagcaccccccatgcacagtccggcccagcaacaacaacagcagcagcagcagcagcagcaacagcaggcctcgccgcatctctcgcttagctcaccgcaccagcagcagcagcagcagcagggacagcaccaaaaccaccaccagcaacaaggtgggggtggcggaggagctggtggaggagctcaactgccgccgcacctggtgaacggaacgatactgaagacggccctaaccaatcccagcgagattgtacatctgcgccaccgcctcgactcggcggtcagttcgtccaaggaccgacagatctcgtacgagcacgccttaggcatgatccagacactgatcgactgcgacgcgatggaggacatagccacactgccgcacttcagcgagttccttgaggacaagtcggagattagcgagaaactgtgcaacatcggcgattccatagtccacaagctggtgtcgtggacaaaaaagttgcccttctacctggagatcccggtggagatacataccaaactactgacggacaagtggcacgagatccttatcctgaccacggccgcctaccaggcgttgcatggcaagcggcgtggcgagggaggaggcagcaggcatggttcgccggcgtcaacgccactgagcacgcccactggtacgccgttgagcacaccgataccctcgcccgcccagccactgcacaaggacgacccggagtttgtcagcgaggtgaactcgcacctgagcacactgcaaacctgcttgaccacgctaatgggccagccgatagcgatggagcagctgaagctggacgtcgggcacatggtggacaagatgacccagatcaccatcatgttccggcgaatcaagctcaagatggaggagtacgtctgcctgaaggtttacatactgctaaacaaagcagaagtggaactggagagcatccaggagcggtacgtccaggtgctgcgctcctacctgcaaaactcctcgccgcagaatccgcaggcgaggctcagtgaactgctctcccacataccagagatccaggctgcggctagcctgctgctcgagagcaagatgttctatgtgcccttcgtgctcaactcggcgagcataaggtag

SEQ ID NO: 14 - protein, 1520 aa, Drosophila melanogaster
MTLSRGPYSELDKMSLFQDLKLKRRKIDSRCSSDGESIADTSTSSPDLLAPMSPKLCDSGSAGASLGASLPLPLALPLPMALPLPMSLPLPLTAASSAVTVSLAAVVAAVAETGGAGAGGAGTAVTASGAGPCVSTSSTTAAAATSSTSSLSSSSSSSSSTSSSTSSASPTAGASSTATCPASSSSSSGNGSGGKSGSIKQEHTEIHSSSSAISAAAASTVMSPPPAEATRSSPATPEGGGPAGDGSGATGGGNTSGGSTAGVAINEHQNNGNGSGGSSRASPDSLEEKPSTTTTTGRPTLTPTNGVLSSASAGTGISTGSSAKLSEAGMSVIRSVKEERLLNVSSKMLVFHQQREQETKAVAAAAAAAAAGHVTVLVTPSRIKSEPPPPASPSSTSSTQRERERERDRERDRERERERDRDREREREQSISSSQQHLSRVSASPPTQLSHGSLGPNIVQTHHLHQQLTQPLTLRKSSPPTEHLLSQSMQHLTQQQAIHLHHLLGQQQQQQQASHPQQQQQQQHSPHSLVRVKKEPNVGQRHLSPHHQQQSPLLQHHQQQQQQQQQQQQHLHQQQQQQQHHQQQPQALALMHPASLALRNSNRDAAILFRVKSEVHQQVAAGLPHLMQSAGGAAAAAAAAVAAQRMVCFSNARINGVKPEVIGGPLGNLRPVGVGGGNGSGSVQCPSPHPSSSSSSSQLSPQTPSQTPPRGTPTVIMGESCGVRTMVWGYEPPPPSAGQSHGQHPQQQQQSPHHQPQQQQQQQQQQSQQQQQQQQQQSLGQQQHCLSSPSAGSLTPSSSSGGGSVSGGGVGGPLTPSSVAPQNNEEAAQLLLLPGTDTHPGHEITGHTPFRTPHALNMERLWAGDYSQLPPGQLQALNLSAQQQQWGSSNSTGLGGVGGGMGGRNLEAPHEPTDEDEQPLVCMICEDKATGLHYGIITCEGCKGFFKRTVQNRRVYTCVADGTCEITKAQRNRCQYCRFKKCIEQGMVLQAVREDRMPGGRNSGAVYNLYKVKYKKHKKTNQKQQQQAAQQQQQQAAAQQQHQQQQQHQQHQQHQQQQLHSPLHHHHHQGHQSHHAQQQHHPQLSPHHLLSPQQQQLAAAVAAAAQHQQQQQQQQQQQQQAKLMGGVVDMKPMFLGPALKPELLQAPPMHSPAQQQQQQQQQQQQQQASPHLSLSSPHQQQQQQQGQHQNHHQQQGGGGGGAGGGAQLPPHLVNGTILKTALTNPSEIVHLRHRLDSAVSSSKDRQISYEHALGMIQTLIDCDAMEDIATLPHFSEFLEDKSEISEKLCNIGDSIVHKLVSWTKKLPFYLEIPVEIHTKLLTDKWHEILILTTAAYQALHGKRRGEGGGSRHGSPASTPLSTPTGTPLSTPIPSPAQPLHKDDPEFVSEVNSHLSTLQTCLTTLMGQPIAMEQLKLDVGHMVDKMTQITIMFRRIKLKMEEYVCLKVYILLNKAEVELESIQERYVQVLRSYLQNSSPQNPQARLSELLSHIPEIQAAASLLLESKMFYVPFVLNSASIR

SEQ ID NO: 15 - protein, 693 aa, Drosophila melanogaster
MGTAGDRLLDIPCKVCGDRSSGKHYGIYSCDGCSGFFKRSIHRNRIYTCKATGDLKGRCPVDKTHRNQCRACRLAKCFQSAMNKDAVQHERGPRKPKLHPQLHHHHHHAAAAAAAAHHAAAAHHHHHHHHHAHAAAAHHAAVAAAAASGLHHHHHAMPVSLVTNVSASFNYTQHISTHPPAPAAPPSGFHLTASGAQQGPAPPAGHLHHGGAGHQHATAFHHPGHGHALPAPHGGVISNPGGNSSAISGSGPGSTLPFPSHLLHHNLIAEAASKLPGITATAVAAVVSSTSTPYASAAQASSPSSNNHNYSSPSPSNSIQSISSIGSRSGGGEEGLSLGSESPRVNVETETPSPSNSPPLSAGSISPAPTLTTSSGSPQHRQMSRHSLSEATTPPSHASLMICASNNNNNNNNNNNNNNGEHKQSSYTSGSPTPTTPTPPPPRSGVGSTCNTASSSSGFLELLLSPDKCQELIQYQVQHNTLLFPQQLLDSRLLSWEMLQETTARLLFMAVRWVKCLMPFQTLSKNDQHLLLQESWKELFLLNLAQWTIPLDLTPILESPLIRERVLQDEATQTEMKTIQEILCRFRQITPDGSEVGCMKAIALFAPETAGLCDVQPVEMLQDQAQCILSDHVRLRYPRQATRFGRLLLLLPSLRTIRAATIEALFFKETIGNVPIARLLRDMYTMEPAQVDK

SEQ ID NO: 16 - DNA, 3049 bp, Drosophila melanogaster
gtcagcccaggcgatccgcatttgcgtccgcagcaggtttccgatttcagaactctgattccagcggcagcgaatcgcgtcggcatctgaacatttgaaaataatctaaaattgcaagtgactttgtgcaccggttacactaaaattgttaacaaatcgccatatattctgaatttaaatttaaagtgcgcagtgcggaatataaatcggagcaaactggatacgttagggttcaaatacttccatcaacggaaaatgggcacagcgggcgatcgcctgttggacattccctgcaaggtgtgtggcgatcgcagctccggcaagcactatggaatctacagctgcgatggctgctccggttttttcaagcggagcattcatcgcaatcggatttacacctgtaaggccaccggcgatctcaagggtcgctgtccggtggacaagacccatcggaatcagtgtcgcgcctgtcgcctggccaagtgcttccagtcggccatgaacaaggatgctgtgcagcacgagcgcggtcctaggaaacccaagttgcacccgcaactgcatcatcatcatcatcatgctgctgccgccgccgctgcagcgcatcatgcagcagccgcccatcaccatcaccatcatcaccaccacgcccacgcagcggccgcccatcatgcggcagtggctgcagcggctgcctccgggctgcatcaccaccaccacgccatgcccgtctcgctggtgaccaatgtctcggcctcgttcaactatacgcagcacatctccacgcatccgcctgctccggcggcgccacccagtggctttcacctgacggccagtggcgcccagcagggaccagctccaccagctggccacctgcaccatggtggagccggacatcagcacgccacggccttccaccatccgggacatggacacgcgttgcctgccccacatggcggcgtgatcagcaatcccggcggcaactcgagcgcaatctccggcagcggacccggctccacgctgcccttcccctcgcacctgctgcaccacaatctgatagcggaggcggccagcaagctgccgggcatcactgccacagccgttgcggcggtggtgtcctccactagcacgccctacgcctcggcggcccaggcgtcgtcgcctagtagcaacaaccacaactactcctcgccctcgcccagcaactccatccagtccatctcgagcattggatcgcgcagcggtggtggcgaggagggcctcagcctgggcagcgagagtccgcgcgtcaatgtggaaactgagacaccttcgccctcaaactcgccgcccctgagtgctggtagcatttcgccagcgcccacgttgaccacctcgtcgggatcgccgcagcaccgccagatgtcgcggcacagcctcagtgaggcaaccacgccgcccagccacgcctctctcatgatttgcgccagcaacaacaacaacaacaacaataataataacaacaacaataatggagagcacaagcagtcgagctacacatccggatcaccgacacccacaacgcccacgccgccaccgccgcgttctggtgtaggttccacctgcaacacggccagcagctccagcggcttcctggagctgctgctcagtccggacaagtgccaggagctcatccagtaccaggtgcagcacaacacgctgctcttcccgcagcagctgctggactcgcggctgctctcctgggagatgctgcaggagacgacggcgcgactgctcttcatggcggtgcgctgggtcaagtgcctcatgccgttccagacgctctccaagaacgaccagcatctgctgctccaggaatcctggaaggagctctttctgcttaatctcgcccaatggactataccgctggatctaacgcccatactggaatcaccgctcatccgcgaacgggtgctgcaggacgaggccacacagacggagatgaagacgatccaggagatcctgtgccgcttccgccagatcacacccgacggcagcgaggttggctgcatgaaggccatcgccctgttcgcacccgaaaccgccggcctgtgcgacgtgcagccggtggagatgttgcaggatcaggcgcagtgcatcctctccgaccatgtgcgactgcgctacccgcgccaagcaacccgcttcggcaggctgttgctcctgctgccctcgctgcgcaccatccgggcggccaccatcgaggcgctgttcttcaaggagaccatcggcaatgtgcccattgctcgactgctgcgcgacatgtacaccatggaaccggcacaggtggacaagtgaaccggccacgcatggccgtcgaaatgaaatcaaaatcgattccctagcacctaagcgccacccatcggtcgtcgtcatatgcgaacttatttgtattccaatgggacccgaatcgtattcagattcactgcggcaggaggcggtccaaatgtggggcggaagctgcagatgctatggttcgcaggacgccatgtaatggaggcgtatgtactaaccgcgctcctccattggcgatgcagtccgcgatgatggcgcactcccacacccacaccagtacccacaccttgatttatcgccggcaatgcgtcggagtctccttactttcgcttcgttttctaacatttgtatccttattttatttcatctttttccacggatttttcgttttgactgcctgggcggcactctttatttatctttcattcgacgttttgtcgtcgcttttctaaaaattccccatgttatttcaacctggcaaggacctcgcaatcccattcccgcgcccttacttacaaatcacttcccatcccacatccagcaattccgtggtttgaattctttcgtgcattgactacgaaataccctttaatcagacaaataaagaatattagttgtaattcttttttctgcaatccagctctaaaacgggtttcttaatcgaaatcgataaatgtaaaaattatacatatcctttaccaacattgtttgccta

SEQ ID NO: 17 - DNA, 1974 bp, Heliothis virescens
ccgagcggcacgcgtgccgcttgatgctaactattctcattgcgatcgtaacataccacgcgctgtcaacacatgttcgaacagtagttatagtttctttggtgactgacacagacgcttaacgcctgtaaacttcttacaccgctcgtatagaacaaaatgtattggatttatgtgcttcctacaatagtgcttgaacaggtgcagcccgcgagcggtagctagtgttgcggccagaccgctagtgaactcggtgactgtgtgaacgttgttgcggttgaacgccgccttagggatttgtaataaacagccgcactgagcggccgagcatgacgatggaccagcagacaggcctcatgtccctcaatatgtccccgtttgatctgagcccaggccccgaagggtctggttcaggtgggggtccctcgagtgcatcgcaacagtacgtgccgcaaggcgccgcgtatcaatgcccccctgaacaacaatctttcggctatgccaatctggatgcttcatatctgtttccaacaggcgccggtggcgaaccaggcgcctacttgcctgcagccggcaccgtgtgtgatcagacagacaccaaggatgtgatcgaagaactgtgtcccgtctgtggagacaaagtcagcggttatcactacgggctactgacgtgcgagtcttgtaagggcttcttcaaaagaaccgttcaaaacaagaaggtgtacacgtgcgtcgctgaacgagcctgccacatagacaaaacacaacggaaacgctgcccattttgccgcttccaaaagtgcctcgatgttggcatgaaactcgaagctgtacgagctgaccgcatgcgcggtggtcgtaacaagttcggccccatgtacaaacgggatcgtgcccgcaaactgcagatgatgcgacagcgacaaatcgcagtgcagacattgcgcggctcacttggtgacagcggcctggtgctcggcttcgggtctccttacgccacagttccagtcaagcaagagatacagatccctcaagtgtcgtctctcacgtcttcacccgagtcgtcgcctgggccggccttactggccgcccagccgcagccaccacagccgccgccgccgccagctcacgacaagtgggaagcacattcgccgcactccgcatctccggacgccttcgcgtttgacgcgccagccacagcagccgcaactccatccagcaccgccgaacccacaagcactgaaaccctgcgagtctcacccatgatacgcgaatttgtacaaactatcgatgatcgcgagtggcagaattcgctatttgggctcttgcagagccagacttacaatcagtgtgaggtcgatctctttgagttaatgtgcaaagtattggaccaaaacttattttcacaagtggattgggcgagaaacaccgtgttctttaagtatctaaaggtcgatgatcaaatgaagctattgcaacactcgtggtcggacatgctggtgttagatcacctacaccagaggatgcacaatggcctgcctgatgagactaccctccacaacggacagaagtttgacctcctttgtctgggactcctgggcgttccatccctggctgatcacttcaacgaactccagaacaaattagcagagctcaaattcgatgtaccggactacatatgtgttaaattcttgcttctattaaatcctgaggtgagaggcatcgtgaatgtgaagtgcgtccgagatggttatcagacagtccaagccgcacttctggactatacgctgtcctgttatcctacgatacaggataaattcgacaaattagtgatggtagtacctgagatccatgccctagcagctcgaggagaagagcacctataccagcggcactgtgcaggccaggcgcccacacaaacactcctaatggaaatgctacacgcgaagcgcaaatcttgaagtctcagtatgg

SEQ ID NO: 18 - protein, 543 aa, Heliothis virescens
MTMDQQTGLMSLNMSPFDLSPGPEGSGSGGGPSSASQQYVPQGAAYQCPPEQQSFGYANLDASYLFPTGAGGEPGAYLPAAGTVCDQTDTKDVIEELCPVCGDKVSGYHYGLLTCESCKGFFKRTVQNKKVYTCVAERACHIDKTQRKRCPFCRFQKCLDVGMKLEAVRADRMRGGRNKFGPMYKRDRARKLQMMRQRQIAVQTLRGSLGDSGLVLGFGSPYATVPVKQEIQIPQVSSLTSSPESSPGPALLAAQPQPPQPPPPPAHDKWEAHSPHSASPDAFAFDAPATAAATPSSTAEPTSTETLRVSPMIREFVQTIDDREWQNSLFGLLQSQTYNQCEVDLFELMCKVLDQNLFSQVDWARNTVFFKYLKVDDQMKLLQHSWSDMLVLDHLHQRMHNGLPDETTLHNGQKFDLLCLGLLGVPSLADHFNELQNKLAELKFDVPDYICVKFLLLLNPEVRGIVNVKCVRDGYQTVQAALLDYTLSCYPTIQDKFDKLVMVVPEIHALAARGEEHLYQRHCAGQAPTQTLLMEMLHAKRKS

SEQ ID NO: 19 - DNA, 2526 bp, Heliothis virescens
cgccgccggccgcacgcgccccatccgcgcggctcatgcgtctcccgccgctcccgctgcccgacatgactgtgaccgagtgccagcgccgactcctggagcccagcgccgcagagccgccgccccccgcgccccccactgattccgacgtgctgctcggcagagttctcgcagagtttgacggcactacggtgctttgccgcgtttgcggtgataaagctagtggatttcattacggcgtgcattcctgtgaaggttgcaagggtttcttcaggcggtctatccaacagaagatccaatatagaccctgcacgaaaaaccaacagtgctcaatcctccggatcaacagaaatcggtgccagtactgccgattgaaaaaatgcatcgcagtaggcatgagcagagatgccgtgcggttcggacgtgttccaaaacgcgagaaagcgcgtatccttgcagcaatgcagcaatcttctacgtcacgagcaaatgagcaggctgcggccgcagagctcgatgatgctccgagattgctggcgcgcgtggtgcgcgctcacctcgacacctgcgagttcacgcgggatcgcgtcgccgctatgcgtgcacgtgctcgcgactgtcccatctactcgcaacccactctggcttgcccactaaacccggcgccagagcttcagtctgagaaggaattctctcagcgatttgcacacgttattcgtggtgtaatagacttcgctggcctcatccccggcttccagcttcttacacaggatgacaagtttacgctgctcaaaagtggactatttgatgcgttatttgtgcggctaatctgtatgtttgacgcgccactcaatagtataatttgtctaaatggccaagtcatgaaacgggactccatacaaagtggtgccaatgcaaggttccttgtggattctacttttaaattcgccgagcgcatgaactctatgaacttaacagatgcagaaatcgcccttttctgcgctatagtgctcatcactccggataggccgggacttcgcaatgtagaactcgtggaaagaatgcatgcgcgtctgaaggcatgcctgcaaactgtggtcgcccagaatagaccggaccgaccaggtttcctcagggaacttatggatacccttcccgacctccgcacattaagcacactccatactgaaaagctagtagttttccgtactgaacacaaagaattactacggcagcagatgtggaccgatgaagaaggtgtgatgtcctggggtgattctggtgccgatgagtcagcacgcagccccattgggtctgtgtccagcagcgagtccggcgaagccgtcggtgactgcggcacgccactcttggccgctacactggccggacgccggcgactcgactctcgtggatccgtagatgaagaagcactaggcgtggcacatctagctcacaacggactcactgtaacaccagtccgcccgcctccacgataccgcaaacttgattcgccaactgattctggcatcgagtcgggaaatgagaagcacgagaggatagttggccccggatcaggatgctcaagcccgcgatcttccctagaggagcacacggatgataggcggccacctccatcagccgatgacatgcctgtgctaaaacgcgtgctagaagcgccaccgctgtaccacaccacttcattaatggacgaggcgtataaacctcacaagaagttccgtgcaatgcgccgtgacaccggcgaagctgaggcccgtgtggtcaggccagcgccctccacgcagccaccgcagcacccgcaccccgcgagcccggcgcacccggcgcactcgccgcgcccactgcgcgcctcgctgtcatccacgcactccgtgctggcgaagagcctgatggaaggcccgcgtatgaccccggagcagctgaagcgcaccgacatcatccagcagtacatgcggcgcggcgaggcgggcgcgcccgacggctgccccatgcgcacgggtgggctgctcacgtgctaccgcggcgcctcgcccgcgccgcagcccgtcatggcgctgcaggtggacgtgtccgacgctgacgcgccccttaacctctccaagaagtcgccgtcgccgccgcgctcctttatgccacagatgttggaggcgtgagatcggcccgcggagccacttgtctccgccaccccaccccgacctactttaaattaaataatctagttaatagtaatcgttcgcagatcgttttaatatattatatgtgataatttgattaggacgcgtgagtgcgtggccggcgccggacgaggagagcgcaatagactgaaaaataatttaagactatataagtgtaatttataaatgcaatgcagcgccggtcgcggtgtgtttgttgtgtgtattgtatgtgcgtatgtgagcgtgctcagtcatttgtttgttaatataatgacaggttgtcgaactaaaataaaagaatcttgtaaaaattaaaaaaaaaaaaaaaaaa

SEQ ID NO: 20 - protein, 711 aa, Heliothis virescens
MRLPPLPLPDMTVTECQRRLLEPSAAEPPPPAPPTDSDVLLGRVLAEFDGTTVLCRVCGDKASGFHYGVHSCEGCKGFFRRSIQQKIQYRPCTKNQQCSILRINRNRCQYCRLKKCIAVGMSRDAVRFGRVPKREKARILAAMQQSSTSRANEQAAAAELDDAPRLLARVVRAHLDTCEFTRDRVAAMRARARDCPIYSQPTLACPLNPAPELQSEKEFSQRFAHVIRGVIDFAGLIPGFQLLTQDDKFTLLKSGLFDALFVRLICMFDAPLNSIICLNGQVMKRDSIQSGANARFLVDSTFKFAERMNSMNLTDAEIALFCAIVLITPDRPGLRNVELVERMHARLKACLQTVVAQNRPDRPGFLRELMDTLPDLRTLSTLHTEKLVVFRTEHKELLRQQMWTDEEGVMSWGDSGADESARSPIGSVSSSESGEAVGDCGTPLLAATLAGRRRLDSRGSVDEEALGVAHLAHNGLTVTPVRPPPRYRKLDSPTDSGIESGNEKHERIVGPGSGCSSPRSSLEEHTDDRRPPPSADDMPVLKRVLEAPPLYHTTSLMDEAYKPHKKFRAMRRDTGEAEARVVRPAPSTQPPQHPHPASPAHPAHSPRPLRASLSSTHSVLAKSLMEGPRMTPEQLKRTDIIQQYMRRGEAGAPDGCPMRTGGLLTCYRGASPAPQPVMALQVDVSDADAPLNLSKKSPSPPRSFMPQMLEA

SEQ ID NO: 21 - DNA, 2951 bp, Heliothis virescens
gcacgagtcaaattaacgttttattacgagtgtgattcataatatcttcaaagacatttgtgtacagcaagggggtgtattgtttcatcttcttataggtatatttagttcaacgaccttgttactgacaggttcatcgttagccactgaactattctatgaaagtggtatatcgtagacgatagataaattaaatccaatgatggagccctcgagagattcagggctaaacttggagggaggttttatgtcgccgatgtcaccgccggagatgaagccagacacggcgatgctagacggcctgcgagacgactccaccccacccccagctttcaagaactaccccccgaaccatcccctaagtggttctaagcacctctgttctatatgtggagatagagcgtcggggaaacattatggagtatacagttgtgaaggttgcaaaggtttcttcaaaaggacggtaagaaaagacttaacgtacgcgtgccgcgaagaacgcaactgcatcatagacaaacgtcagaggaacagatgccagtactgcaggtaccagaaatgtctcgcgtgcggcatgaagagggaagcggtgcaggaggagaggcagagggccgccagaggtacggaggatgcacatccgagcagctcggtgcaggtacaggagttatcaatcgagcggttgctggagatggagtcactggtagctgatcccagcgaagagttccagttccttcgtgtgggacccgacagcaatgtgccacctaagttccgcgcccctgtctccagcctttgtcaaataggcaacaaacaaatagcggcgctagtggtgtgggcgcgcgacatcccgcacttcagtcagctggagatggaagatcagatcctgctcatcaaaggctcctggaacgaactgctgctcttcgctatagcgtggcggtctatggagttcctgacagaagagcgagatggcgtggacggcactgggaacagaaccacatcgccgccacaactcatgtgcctcatgcctggtatgacgctgcaccgcaactcagcgctgcaggcgggcgtggggcagatcttcgaccgcgtgctgtcggagctgtcgctgaagatgcgcaccctgcgcgtcgaccaggccgagtacgtcgcgctcaaggccatcatactgctcaacccagatgtgaagggactgaaaaacaggcaagaagtggaagttttacgagaaaagatgttcctgtgcctggacgagtactgccgccgctcgcgcagttcggaggagggtcggttcgcggcgctgctgctgcgcctgcccgcgttacgttccatttcactcaagagcttcgagcacctgttcttcttccacctggtggccgacaccagcatcgccggctacatccgcgacgcgctgcgcaaccacgcgccgcccatcgacaccaacatgatgtaaccggcacacggtgttactacatctggcaactaaacgaacgcgttcgacaagcgatatcgcgaccgaatgtgcagctacataacgaactattgtctaatattgttgtttgttgaaagactcatggcaattactgagggggatttttaatcaacgatggtataactatgctggaaataaatgaaatatgccatcggtagtaataaagtagtgctaaagcagttcaaatctatttctcaaaatggaaactgctatgagttttttgacaaacttttcgtctctatttcataaaattctatcattatacgaagctaccatttgccggtcaaaggttcgaggttaatgtcctggctacggtcgctaattagagtttattatatggaaagttagctttagcagacgtggggagggcgtgtcgaagtggttatgaacattttcgaggtcttagattataatcttgaaacctcacattatgtgcgtgcgtgtaaccgctgcgcgacctctaacgcgatgtaggtgtttatcgaataaacggtaccgaatacatttgttgctataatttgtaggaattttggtatacgtttcgacggataaaccttataatttcggtaaatttacaaatcaggcctttatttttcgagacagcagacttgatgttatgttagctctaaagatcaaatagttaacgttggtgaaaaccttcagctataagtacctgggttataaatagcccttattgtaaaggccctttatgttatgttcatgtttgagtacgagcacatcaccgttgtatgaagcgcgggcacgcgacggcagtcgtgtactaggtgtccgtgtgggcacggtatcgacacacgacgttaggtagtgtaaatggggccgccttttttattacgtagaatatgattaacgtaaattagtcattcatttattagggaattttaatcgtacaggtaggtgtccatagtgaattaatataatgttagactaatgtgtaattttaacatttaccaaatctctcttcaaagcacgtacattaattattacgtatatttaatggattgactatgtgtccactgagccagtgataaaaatcatgttgattcgtaaaaacgaaaatgaaagaaaaatatacaaaattatgcttcatgtatttcccgtattatacacatgttggtgctttaacatgcattatttacatattatgcatatcaaatatgcatagtaatccttaaatttaatgttacttgataaatccttattttcattacgattagtatagaaagttataaaaactgtaaatatatctttgcatgtacaatgtatttttattatgtttagacgagtaacttatattgcatattaatatatacctcaccagtctcgggtggtgggactaaacagatagaatggcctacacgctacatttataaattataacttgtatttctactgacaggccactttatttttgtaccttgtgagtgccacgcattttccaattcgctat

SEQ ID NO: 22 - protein, 420 aa, Heliothis virescens
IDKLNPMMEPSRDSGLNLEGGFMSPMSPPEMKPDTAMLDGLRDDSTPPPAFKNYPPNHPLSGSKHLCSICGDRASGKHYGVYSCEGCKGFFKRTVRKDLTYACREERNCIIDKRQRNRCQYCRYQKCLACGMKREAVQEERQRAARGTEDAHPSSSVQVQELSIERLLEMESLVADPSEEFQFLRVGPDSNVPPKFRAPVSSLCQIGNKQIAALVVWARDIPHFSQLEMEDQILLIKGSWNELLLFAIAWRSMEFLTEERDGVDGTGNRTTSPPQLMCLMPGMTLHRNSALQAGVGQIFDRVLSELSLKMRTLRVDQAEYVALKAIILLNPDVKGLKNRQEVEVLREKMFLCLDEYCRRSRSSEEGRFAALLLRLPALRSISLKSFEHLFFFHLVADTSIAGYIRDALRNHAPPIDTNMM

SEQ ID NO: 23 - DNA, 645 bp, Heliothis virescens
gcacgaggtggcgaccgctttaccggcaaacattatggagcctcgtcctgcgatggctgcaagggcttcttcaggcgaagcgtcaggaaaaatcatctgtacacatgcaggttcagcagaaattgtgtggtggacaaagacaagagaaaccagtgcagatactgtaggctaaggaaatgcttcaaggcgggcatgaagaaagaagcggtgcagaacgagcgggaccgcatcaactgccgaagaccttcgtatgaggagccgacgcaggccaacgggctgtcggtagtgtcgctactcaatgctgaactgctcagtaggaaggttattgatgagacaaacaacgtgacagacgcggagatcaacaaccgcaagctggctaagatcaacgacgtgtgcgacagcatcaagcagcagctgctcatactggtcgagtgggccaagtacatacccgccttcactgagctgcatcttgatgatcaggtggcgctgctgcgcgcgcacgcaggcgagcacctgctgctgggctgcgcgcgccgctcgctgcacctcagcgacatcctgctgctcggcaacaactgcatcatcaccaagcacaatatcgatggccgcatggacatagacatcagcatgatcggcatgcgcgtg

SEQ ID NO: 24 - protein, 215 aa, Heliothis virescens
ARGGDRFTGKHYGASSCDGCKGFFRRSVRKNHLYTCRFSRNCVVDKDKRNQCRYCRLRKCFKAGMKKEAVQNERDRINCRRPSYEEPTQANGLSVVSLLNAELLSRKVIDETNNVTDAEINNRKLAKINDVCDSIKQQLLILVEWAKYIPAFTELHLDDQVALLRAHAGEHLLLGCARRSLHLSDILLLGNNCIITKHNIDGRMDIDISMIGMRV

SEQ ID NO: 25 - DNA, 716 bp, Heliothis virescens
  misc_feature (1)..(716): n is a, c, g, or t
gcacgagtgcaagggcttctttagacgattacaaagcacagtggtgaactaccaatgtccacggaataaagcctgcgtggtggaccgcgtcaacaggaatcgctgccagtactgcagattgcagaaatgcctcaaacttggaatgagccgtgatgcggtgaagtttggacggatgtcgaaaaagcaacgtgagaaggtggaagatgaggtgagattccatcgtgcacagatgagagcgcagactgacacagcgcccgattcagtatacgacgcccaacaacagacgccgagctcaagcgaccagttccatggtcactacaatggctacccgggctacggatccccattgtcctcatacgggtacaacaacgcgggcccagcattacagtccaacatgggcggcatacagccccaaccgccccaacagcagccctacgacgtgtctgctgactacgtggactccaccacagcttacgagccgaaacagaaccggggattcttggaccctgattntattagtcatgcggagggtgacatcagcaaagttctggtgaagagtttggcagaagcacatgcgaataccaaccttanactggnantcattcacgagatttcagaaagccacaaagacgtcttcccaagctttnctatactatagntcctatgacgtaccgaaaggnaaaaagggaaaaaaatattttttttttggggggggc

SEQ ID NO: 26 - protein, 218 aa, Heliothis virescens
  misc_feature (1)..(218): Xaa is any amino acid
HECKGFFRRLQSTVVNYQCPRNKACVVDRVNRNRCQYCRLQKCLKLGMSRDAVKFGRMSKKQREKVEDEVRFHRAQMRAQTDTAPDSVYDAQQQTPSSSDQFHGHYNGYPGYGSPLSSYGYNNAGPALQSNMGGIQPQPPQQQPYDVSADYVDSTTAYEPKQNRGFLDPDXISHAEGDISKVLVKSLAEAHANTNLXLXXIHEISESHKDVFPSFXIL

SEQ ID NO: 27 - DNA, 5206 bp, Drosophila melanogaster
ggctcgcccattggagggcccctgtcctgtggcagcagcttgcccagcttccaggagacctactccttgaagtacaacagcagcagcggtagcagcccccagcaggcgtcctcctcctccaccgccgcccccacgcccactgaccaggtgctgaccctcaagatggacgaggactgcttcccgcctctgtccggcggctggagtgccagtccgcccgccccctcccagctccagcagctgcacaccctgcagtctcaggcccagatgtcgcatcccaacagcagcaacaacagcagcaacaacgcgggcaacagccacaacaacagtgggggctacaactaccacggccacttcaatgccatcaatgccagcgccaatctgtcgcccagctcctcggccagttccctctacgaatataatggtgtttccgcagcggacaacttctacggacaacagcagcagcagcaacagcaaagctatcagcaacataactacaactcgcacaatggcgagcgttactcgctgcccacgtttcccacgatttcggagctggctgcggccactgctgctgtcgaagctgcggcggcggccacagtgggcggtccgccgccagtacgccgagcatcgctgccggttcagcgaaccgttctgccagccggctccacggcgcagagccccaagctggccaagatcacactgaaccagcggcactcacatgcccatgcccatgccctacagctcaactcggcacccaattcggcggcaagttcgccagcgagtgcggatctgcaggcgggccgtttgctccaggctccgtcgcagctgtgtgctgtttgtggcgacaccgccgcctgtcagcattatggagtgcgaacctgcgagggatgcaagggatttttcaagcggaccgtgcagaagggctccaagtacgtctgcctggcggacaagaattgcccggtggacaagaggcgccgcaaccgttgccagttctgccggttccagaagtgcctggtcgtaggcatggtcaaggaagtggtgcgcaccgactcgttgaagggtcgccgcggaagactgccctcaaaaccgaaatcgccccaggagtcgccaccatcaccacccatctcgttgatcacggccctggtgcgcagccatgtcgacacgactccggatccctcgtgcctggactatagccactacgaggagcagtcgatgagcgaggcagataaggtgcaacagttttaccagctgctgaccagctccgtggacgtgatcaaacagttcgccgagaagattcccggctacttcgatctcctgccggaggatcaggagctgctcttccagagcgcatcgctggaactgttcgtcctgcggctggcctatcgcgccaggatcgatgacaccaagctgatcttctgcaacggcacggtgctccaccgcacccagtgcctgcgctctttcggcgagtggctcaacgacatcatggagttcagccgcagcctgcacaacctggagatcgacatctccgccttcgcctgcctctgtgccctaaccctcatcacagaacgccatggcctgcgggagccgaagaaggtggagcagctccagatgaagatcattggcagtctgcgcgaccacgtcacctacaatgccgaggcccagaagaagcagcactacttcagccgcctgctgggcaagctgccggagctgaggtccctgagtgtccagggactgcagaggatcttctacctgaagctggaggacctggtacccgcaccagctctcatcgagaacatgttcgtcaccacattgcccttctagaggcgatcatcaagcgtatcatcacaacttgcttccttaaaactagcccctaagtatgcctcctaggatatacagagaaaggaccccataggacggacgcaactagctttagtagaaccctgaaataaataaatctcacaacagcaaaaacaaaaccgaaccgaacagaaatgaagcgaatagcagacccaggccatatctttagtgtagagctaggtagttaccgggacagcccggctccttcgataattacggacatgcatatttgagagggggtttccagtgcacagcctatggctcctgcgtgactcgtcagcacgcgagctccaacttgttgacgttaattgttaaattgtttaatttcaactgtcaaaaccggaatcaacggccgggcacgcaatggcaacactttctatccccggacttcgaagcctgctcaacattcggcactacggacggacaaacaacggacagaaacagaactcactcttgctctcttgccttttactaacttctagtcaattgatttaggcgaatcaaataaataaataaataaaataagggcgtgcagcagtagtgttatataatttatatccagaccccagcggttctcttcaaggaaatcccccaatgagttgcacaaattgggataaagtacgatagcctattattcttatatttcttttaaaagctcgaagatagatgagaactgtgttggaaattccactatcatatcatatagttgctataagccgtgcttgccctaagctaagttagacccgcataaagttgatagcccaaccaagtatttcggttatttcctagactaaggtcctaatagttataggctaagactattctgttcgattatcaatgcaccaaacagtgcacaatgagagtataagtaccttcttgtgatgattgtgtctgacacagagagagttgcacacaagcacacaaactagccgataagttactaaatacgatctaatatctaatatatataatataatataatatatataagtccaagtattcggaaatccaagaacccttgcataaccgcagttcgtacgttccaaacgagaaaagaactttatttaatcctagacccactcatctaagcttctaaagaatcgtatgtggatcgttggatctgtctctctatatatgtgtgtgtgtgttatctcgatagaaaacccctctatgtgattttgtgatagattggcattgaactctatatatttatatatatatatgtctataatatatatacacgcataaatatatatttttatgtctaacttttgtatggtttattttatacgtaccacttttctttgataacaaaaagtaaaaaactcgttagatagcaaatatttcaaaggtatgttacgaggacttttcaaaggtatgttcgaggacttttcaaagtaccagtctttagcgactttccaattaacgttcgtattaacgaaagacagattttctatgtgttaaattgaaagacttctataactataactaaatgcaagctaagagcaaaaacacaaatccacaaatccccaaagtgaataacatatctcttcaagctttcgagtgctcggaacacgtagaaccgaaacccaagtgttactaaatccatttaataatcgcaagccgggggcgtcggcgtggttaatacgttctcattacctatacaatttagatagatcattattaaattattgtacatgtagcacatgaaatgttcgacaactagattttgtaccatcttaaagaagaacctaggccaagctaaactaagtataaactatgatctgcatgcggctagctgtagctatgagaaatatacctgcgtggatctaagtgaaatgggacactttgaatttagatatgaaacgttctaaacgcgacgactaactctcccaactgcgaactctaccaattaagagaaattcccagaaaatgtgtcaggatttcaaagcgtcccatctcacttgaacccacccaatcaacaaatacaaatcctagggaagttgagaggttcagcaaccatagagcaatatttcataagaaaacgcaccttaaattaccgaaaaacatagattaacctgatcttgtaacgtttgggagcgataataagccaggattaaacaggaacagttaggtgaccaaatcagttcgaaacgaagatgatagatagttcggttcgaaaccctaaacgcgatgccattttagccgttacaacattggatatcaatcatgcacatgaatatgaatatgaatatgaatattatagagatatatctagctataggaacctactttgtacctacacgacatggaaacatcaaacctacatgcatatttacacacatatattttgaatagagcgacgacttttacaagttgcgtagaaagctatagctatagcttgatatggccatcccagagcgagcatatacatatattttgggttattgttcttttgtaattttataaatgcatacatatttatgtactacgtgaatgtcaagtgtggattcatatttttgagatacagctacaaaaacgaaacaaaagaaaataaaacaaaacagaagagtaaacggtgaaattttttcgatgaaacaattttaaatgagaactttttaatattgctattaaaggatatacatatacacactaacatacaatatatattttactatgtaacggatagaattaagctagatgcagcgcataaagctttatacaacaaattgaaaagcaacagaagaaattggcacaaattaaatttatatagcataattagacgtccttcgcaagataatgttattgctaataagagcgtcaatcggtacatcgggcgctatttcccactacacccccaaccacacaatagataacctaagcaatgtatgtacattagctatgtatatccagcccacttatgcgcctactactagaaatgcagaaagcagaaagagaggtgaaacctatagacgctatcacaaatgtctatctgatagacatcggtactaccaatgctatattgccagttgtgtaatttactcttatttgatcgtttcatttaccagttaagaacccaaatcatataagtgttatgatggaagaactataacttgcaattcaattaactctgcatacgataacaagcaaagcgaatcatttcatttcgatttaatctttaattatatatacttaaacgatgtaagcccaaaacaaacgttttttctatatctgtcttttgagcaaattagttatacgcaaaaccaaaccgtatttacataaatgtatacaaaacaaatcgtatattttcattggtttgaaataaatacataaaacaaaaaaaaaaaaaaaa

SEQ ID NO: 28 - protein, 551 aa, Drosophila melanogaster
MDEDCFPPLSGGWSASPPAPSQLQQLHTLQSQAQMSHPNSSNNSSNNAGNSHNNSGGYNYHGHFNAINASANLSPSSSASSLYEYNGVSAADNFYGQQQQQQQQSYQQHNYNSHNGERYSLPTFPTISELAAATAAVEAAAAATVGGPPPVRRASLPVQRTVLPAGSTAQSPKLAKITLNQRHSHAHAHALQLNSAPNSAASSPASADLQAGRLLQAPSQLCAVCGDTAACQHYGVRTCEGCKGFFKRTVQKGSKYVCLADKNCPVDKRRRNRCQFCRFQKCLVVGMVKEVVRTDSLKGRRGRLPSKPKSPQESPPSPPISLITALVRSHVDTTPDPSCLDYSHYEEQSMSEADKVQQFYQLLTSSVDVIKQFAEKIPGYFDLLPEDQELLFQSASLELFVLRLAYRARIDDTKLIFCNGTVLHRTQCLRSFGEWLNDIMEFSRSLHNLEIDISAFACLCALTLITERHGLREPKKVEQLQMKIIGSLRDHVTYNAEAQKKQHYFSRLLGKLPELRSLSVQGLQRIFYLKLEDLVPAPALIENMFVTTLPF

SEQ ID NO: 29 - DNA, 4778 bp, Drosophila melanogaster
gaaaaaacaaacattttgctacttcgtcgcaggcgggactgtgttgcgtcgtgtgatcgctagagcggttgtggaatcggattcgagcgcaaaacaccgttcatgctgtgaaaaatccgatatttgtcgtgcaataatttcctcgattggcatcaagtggcttccagtcgggtacatattgcacaagaaatgttatacgcataatgtgcacgcaaattaaacgaattctctatgaaaatgtgactagaatgtgagtcgaacaaaacgagtaaaacgtgaaatcccaactggcttttgggtaacaaatcttatcaacacagcaacggaaatacattaaaatcttgatagactgagaaagggacaattggaatacttttagttatttttaaatgttttacaacacaatggaactgcatcaacgacacctctcaaacttttacaaattgcacaactgagaaatagtctttgataaataaataaaatataagaaatcgctactgaaacaagatgccaaacatgtccagcatcaaagcggagcagcaaagcggtcctcttggaggaagtagcggctatcaagtaccggtcaacatgtgcaccaccacagtcgcgaatacgacgaccactttgggaagctccgccgggggagccactggctcccggcacaacgtctccgtgacaaacatcaagtgcgaactagacgaactaccgtcaccgaacggcaacatggtgccggttatcgcaaactacgttcacggtagcttgcgcattccactcagtggacattcaaatcatagggagtccgattcggaggaggagctggcaagtattgagaacttgaaggttcggcgaaggacggcggcggacaaaaatggtcctcgtccaatgtcctgggagggcgagctgagcgatactgaggtcaacgggggcgaagagctgatggaaatggagccaacaattaagagtgaggtggtccctgctgttgcacccccacaacccgtctgcgcactacaaccgataaaaacagagctagagaacattgcaggcgagatgcagattcaagggaagtgttacccccagtccaacacacaacatcacgctgccacaaaattaaaagtggccccgacgcaaagtgatccgatcaatctcaagttcgaaccgcctctgggagacaattctccgctactggctgcacgtagcaagtccagcagtggaggccacctaccactgccaacgaatcccagtcccgactccgccatacattccgtctacacgcacagctccccctcgcagtcgcctctgacgtcgcgccacgccccctacactccgtctctgagccgcaacaacagcgacgcctcgcacagtagctgctacagctatagctccgaattcagtcccacacactcgcccattcaagcgcgtcatgccccacccgccggcacgctctatggcaaccaccatggtatttaccgccagatgaaggtggaagcctcatccactgtgccgtccagtgggcaggaggcgcagaacctgagtatggactctgcctctagcaatctggatacagtgggcttaggatcttcgcaccccgcatctccggcgggcatatcacgtcagcagttgatcaactcgccctgccccatctgcggtgacaagatcagcggatttcattactttattttctcctgcgagtcttgcaagggcttcttcaagcgcaccgtgcaaaatcgcaagaactacgtgtgcgtgcgtggtggaccatgtcaggtcagcatttccacgcgcaagaaatgtccagcctgccgcttcgagaagtgtctgcagaagggaatgaaactagaagcgattcgggaggaccgaacccgtggcggccgctccacataccagtgctcctacacgctgcccaactcaatgcttagtccgctgcttagtcctgatcaagcggcagcagctgccgccgcagcagcagtggcaagtcagcagcagccgcaccagcgactacatcaactaaatggatttggaggtgtacccattccctgctctacttctcttccagccagccctagtttggcaggaacttcggtcaagtcggaagagatggcggagacgggcaagcaaagcctccgaacgggaagcgtaccaccactactgcaggaaatcatggatgtagagcatctgtggcagtacaccgatgcagagctggcccgcatcaaccaaccactgtccgcattcgcctctggcagctcttcgtcgtcgtcatcgtcaggtacatcctcaggcgcccatgcacaactcaccaatccactactggctagtgctggtctctcgtccaatggcgagaatgccaatcctgatcttatcgctcatctctgcaacgtggctgatcaccgtctttataaaatcgtcaaatggtgcaagagcttgccgctttttaagaacatttcgatcgatgaccaaatctgcttgctcattaactcgtggtgcgagctgttgctcttctcctgctgttttagatcaattgatactcctggagagattaaaatgtcacaaggcaggaagataaccctatcgcaggccaaatcaaatggcttgcagacttgcattgaacggatgctcaacctaacagatcacctgaggcgattgcgcgttgatcgctacgaatatgttgccatgaaagttattgtgctgttgcagtcagatacgacagagttacaggaagcggtaaaggtgcgcgagtgtcaggaaaaagctttgcagagcttgcaagcttacaccctggcgcattatcctgacacgcaatccaagtttggggagcttttgctacgcattcctgatttgcagcgaacgtgccagcttggcaaggagatgttgacgatcaagactcgcgatggagctgatttcaatttgctaatggagcttttgcgcggagagcattgacaattgataactaagacggaaatcttttaccattggcaaaacaagtttcacatatttagtattagatatatatattctatagataagatccttactgtaagttctgaaaacatgtgcctaaaaaccaaagccacgatagcagtcacatcaggcccactggtcgagattaaatccaagagcaagattgccaaatttttacaccaatatatattttgatatgagccatgtgcagggcctcagatcgctgttgttgtcggctaaagtttcagtaaggaaagtatatattgattttgctatttatacatatttgacttatgtatagtgtaaactaaagcacacatggaaaatgaaaagactaaacaaatttatttaaaaattacttttactattatagaaaaaggggaaaaataaaaaacacaaaggcagagaagaaaatttagttacaacaggtagcgacatttttatattttcttatataaggaaatattcaatgtattttaaatataaagccaaacccgatttggtttggggaagagctactgaaatttttgatatctatatattcattactagaagacgaatgaatgtatccaatgtttaaatgttgtagcgtttagttttagtgcaatttcacacatgtctacatacatgaatattcagcgagatatgtctgcaaactattataaagcaaaagaccactcgaaatcgccatcactggttggctaagactattccagttatgctgtttgttgcataaaaaaccacaactacgtacatcaataaaatgtataattttttattggagttttagatttgtattaacttcttccttataattacgattattattattattactaattttatgattattgtgtaacactgacttaaatagctgaaacaaatcctgcaacaggatttaaaacacctgaatacacaaaacattataacatgaatacattttgcttatggcctagatagtttgatatgtactttgatatgtatgcatgtgtctatatgtgagtgcgtccatacaaattcctgtcccaccagaaaaatcacacgcaataaaaaattccaaaatactaagctcgtatctacaaagaaagattaaaagacaaattgatgaataggaatatgttgccggaagtccaagagatttggctgaaagtatcgacaaattttcaacacatcgttcatggatattgtgctaacactctcagtttgaaaatcattttctgttaaactttctatataataagttctccattcgattttgtatttacaatttgtttctttaattttcctttatcagttgtatctatgaaacatgaggatcacagttcatattgatcgtgttcttctgccgtacaccgcttctgtccgttaatgtaaaccataagtataaatgaaattagttaaatgtttatttataaataaagcgctataataaatttcaatacatttatcatagttaactgattaagaccactgaaatcaaaaatattttatttactaagcaaagcacacgcaaacaatttataatgtttattacgttaacaacaaactcatttttaataattctttatgaatacacaaagttacgcaattttccctctaggcgcattgcttaaatagttaaagaaaaataataaacccatagcgcaatatttaatgtaaaacagttttccttgcgtgtgatgtttcctctagctacgtacaaattcatcatttattaaatttaaaactcaattttgcttttaaataaatttaataagtaaaattcaacaataattgatatacaattgtcaatgcaatattttgtaataaaaatgcgaaaaatc

SEQ ID NO: 30 - protein, 808 aa, Drosophila melanogaster
MPNMSSIKAEQQSGPLGGSSGYQVPVNMCTTTVANTTTTLGSSAGGATGSRHNVSVTNIKCELDELPSPNGNMVPVIANYVHGSLRIPLSGHSNHRESDSEEELASIENLKVRRRTAADKNGPRPMSWEGELSDTEVNGGEELMEMEPTIKSEVVPAVAPPQPVCALQPIKTELENIAGEMQIQGKCYPQSNTQHHAATKLKVAPTQSDPINLKFEPPLGDNSPLLAARSKSSSGGHLPLPTNPSPDSAIHSVYTHSSPSQSPLTSRHAPYTPSLSRNNSDASHSSCYSYSSEFSPTHSPIQARHAPPAGTLYGNHHGIYRQMKVEASSTVPSSGQEAQNLSMDSASSNLDTVGLGSSHPASPAGISRQQLINSPCPICGDKISGFHYFIFSCESCKGFFKRTVQNRKNYVCVRGGPCQVSISTRKKCPACRFEKCLQKGMKLEAIREDRTRGGRSTYQCSYTLPNSMLSPLLSPDQAAAAAAAAAVASQQQPHQRLHQLNGFGGVPIPCSTSLPASPSLAGTSVKSEEMAETGKQSLRTGSVPPLLQEIMDVEHLWQYTDAELARINQPLSAFASGSSSSSSSSGTSSGAHAQLTNPLLASAGLSSNGENANPDLIAHLCNVADHRLYKIVKWCKSLPLFKNISIDDQICLLINSWCELLLFSCCFRSIDTPGEIKMSQGRKITLSQAKSNGLQTCIERMLNLTDHLRRLRVDRYEYVAMKVIVLLQSDTTELQEAVKVRECQEKALQSLQAYTLAHYPDTQSKFGELLLRIPDLQRTCQLGKEMLTIKTRDGADFNLLMELLRGEH

SEQ ID NO: 31 - DNA, 2561 bp, Drosophila melanogaster
ctacgcaaaataaaacgtacatgaaatgttattagaaatggatcagcaacaggcgaccgtacagtttatatcgtcgctgaatatatcgccgttcagcatgcagctggagcagcagcagcagccctccagtcccgctctggccgccggtggcaacagcagcaacaacgcggccagcggtagcaacaacaacagcgccagcggcaacaacaccagcagcagcagcaacaacaacaacaataacaacgacaatgatgcacacgttctaacgaaattcgagcacgaatacaatgcctacacgttgcagttggccggaggcggtgggagtggcagcggcaatcagcagcaccacagcaaccacagcaaccacggcaaccaccaccagcagcagcagcaacaacagcaacagcagcagcaacatcagcagcagcagcaagaacactaccagcagcaacagcagcagaatatcgccaacaatgccaatcaattcaactcctcgtcctactcgtatatatacaatttcgattcacagtatatattccctacaggctaccaggacaccacctcctcacactcgcaacagagcggaggaggcggtggcggcggcggtggcaacctgctaaacggcagctccggcggcagctccgccggcggtggctacatgctgctcccccaggcggccagctccagtggcaataatggcaacccgaatgccggccacatgtcctccggttccgtgggcaatggcagcggaggcgctggcaatggcggagcgggtggcaactccggtcccggcaatcccatgggcggcacgagcgccacgccgggacacggcggcgaggtgatcgacttcaagcacctgttcgaggagctttgccccgtgtgtggcgacaaggtgagcggctaccactacggcctgctcacctgcgagtcctgcaagggcttcttcaagcgcaccgtgcagaacaagaaggtctacacctgtgtggcggagcggtcgtgccacatcgacaagacgcagcgcaagcggtgtccctactgccgattccagaagtgcctcgaggtgggcatgaagctagaggctgttcgagcggatagaatgcgtggtggacgcaacaaattcggacccatgtacaaacgggatcgcgcgcggaagttgcaagtgatgcggcagcggcagttggcgctgcaagcgctgcgcaactcgatgggtccggacatcaagccaacgccgatctcgccgggctaccagcaagcatatccaaatatgaacattaagcaggaaattcaaatacctcaggtatcctcactcacccaatctccggactcgtcgcccagccccatagcaattgcgttgggacaggtgaacgcgagcacgggcggtgttatagccacgcccatgaacgccggcactggcggcagtgggggcggtggtctgaacggaccaagttccgtgggcaacggcaatagcagcaacggcagcagcaacggcaacaacaacagcagcacgggcaacggaacgtccggaggaggaggtggcaataatgcgggcggcggaggaggaggaaccaattccaacgatggcctgcatcgcaacggcggcaatgacagcagcagttgccacgaggctggaataggatctctgcagaacacggccgactcgaaattgtgcttcgattctggcacacatccatcgagcacagccgacgcgctaatcgagccattaagagtctcaccgatgattcgtgaatttgtgcaatcgattgacgatcgggaatggcagacgcaactgtttgccctgctgcagaagcaaacctacaaccaggtggaagtggatctcttcgagctgctgatgtgcaaagtgctcgaccagaatttgttctcgcaagtagactgggcacggaacaccgtcttcttcaaggatctgaaggtcgacgaccaaatgaagctgctgcagcattcctggtcggacatgcttgttctggatcacctgcatcatcgaatccataacggcctgcccgacgagacgcaactgaacaatggtcaggtgttcaatctgatgagtctgggtttgttgggagtgccacaacccggcgattacttcaacgagctgcagaacaagctgcaggacctgaaattcgatatgggcgactatgtctgcatgaaattcctaatcctgttgaatccaagtgtacggggtattgtcaaccggaagaccgtatccgagggacatgataatgtgcaagccgctttgctggactacaccctcacctgctatccgtcagtgaatgacaaattcagagggctagttaacatcttaccggaaatccatgccatggccgttcgcggcgaggatcacctgatcacctgtacaccaagcactgtgccggcagtgcgcccacccaaacgctgctcatggagatgctgcacgccaagcgcaagggatagaggtcgggagaacgtgacacggaatacttaatcatttatgaaatgtaaataacaaggcgggaaggccctcggggcaaccgggacatggaaggcgaacgaaggatacagcagaattc

SEQ ID NO: 32 - protein, 816 aa, Drosophila melanogaster
MLLEMDQQQATVQFISSLNISPFSMQLEQQQQPSSPALAAGGNSSNNAASGSNNNSASGNNTSSSSNNNNNNNDNDAHVLTKFEHEYNAYTLQLAGGGGSGSGNQQHHSNHSNHGNHHQQQQQQQQQQQQHQQQQQEHYQQQQQQNIANNANQFNSSSYSYIYNFDSQYIFPTGYQDTTSSHSQQSGGGGGGGGGNLLNGSSGGSSAGGGYMLLPQAASSSGNNGNPNAGHMSSGSVGNGSGGAGNGGAGGNSGPGNPMGGTSATPGHGGEVIDFKHLFEELCPVCGDKVSGYHYGLLTCESCKGFFKRTVQNKKVYTCVAERSCHIDKTQRKRCPYCRFQKCLEVGMKLEAVRADRMRGGRNKFGPMYKRDRARKLQVMRQRQLALQALRNSMGPDIKPTPISPGYQQAYPNMNIKQEIQIPQVSSLTQSPDSSPSPIAIALGQVNASTGGVIATPMNAGTGGSGGGGLNGPSSVGNGNSSNGSSNGNNNSSTGNGTSGGGGGNNAGGGGGGTNSNDGLHRNGGNDSSSCHEAGIGSLQNTADSKLCFDSGTHPSSTADALIEPLRVSPMIREFVQSIDDREWQTQLFALLQKQTYNQVEVDLFELLMCKVLDQNLFSQVDWARNTVFFKDLKVDDQMKLLQHSWSDMLVLDHLHHRIHNGLPDETQLNNGQVFNLMSLGLLGVPQPGDYFNELQNKLQDLKFDMGDYVCMKFLILLNPSVRGIVNRKTVSEGHDNVQAALLDYTLTCYPSVNDKFRGLVNILPEIHAMAVRGEDHLITCTPSTVPAVRPPKRCSWRCCTPSARDRGRENVTRNT

SEQ ID NO: 33 - DNA, 5970 bp, Drosophila melanogaster
acttactagtgaaaaacatgataataaacaacttgccaaaaaaaatccaatgaaattgacacttatgttaaaaaaataggtgagattgtaaccgttgatgtacacttacgaagtacgtaacaagttcatgaactgatttcgtgagcaggtctctccataatcgccgtatctgtgggatcgcgcgctcctgctcgcactcgctgggtggatggcagcacatgttcgaagtgcgagagagtgcaaagcggagagcgccgacgtcgacgccgaaaaaactgaacaagatccgccgcgaatgttgattttcctttcattgactaactgccactcgcagcgcgcagatcgtcggctccgcttgttccgttccgttcgtttcgtttcgtttcgttcgatctacttcgagtcgcgagttttaagcagtgtagtgagtgccccgtgaaaaggataacccaaaaagtgatttctactattttccaatagtttttatcagtgtgaagaaaacatgtaaacttggctcaaaaagggctttaaaagatacaaagcttcaatgcgaaggataaaataatatcgcaccagtgcttcaaaaaccaaaactatgcctaaggctggaaatttaaattaaaatttttttaataaatattccaaaaatattgcccctgaaaagtgttgataaacccccaaccgagcaaaatgttaatgtccgcggacagttcagatagcgccaagacttctgtgatctgcagcacggtgagtgccagcatgctagcaccaccagctccagaacagcccagcaccacagcaccacccattttgggggtaacaggtcgatctcacctggaaaatgccctgaaactaccgccaaacacaagtgtttcggcttactaccagcacaacagcaagctgggcatgggccagaattacaatccggaattcaggagcctggtagcacctgtcacagatctggatactgtgccacccacaggtgtgaccatggcgagttcttcgaattctcccaactcctccgtcaagctgccccacagcggcgtgatctttgtcagcaaatcgagtgccgtcagcaccaccgatggtcccactgcagtgttgcaacagcagcagccgcagcagcaaatgccccagcacttcgagtccctgccccaccaccacccccagcaggaacaccagccacagcagcagcagcaacaacatcaccttcagcaccacccacatccacatgtgatgtatccgcacggatatcagcaggccaatctgcaccactcgggtggtattgctgtggttccggcggattcgcgtccccagactcccgagtacatcaagtcctacccagttatggatacaactgtggctagttcggtaaagggggaaccagaactcaacatagaattcgatggcaccacagtgctgtgccgcgtttgcggggataaggcctccggtttccattacggcgtgcattcctgcgagggttgcaagggattcttccgccgctccatccagcaaaagatccagtatcgcccgtgcaccaagaatcagcagtgcagcattctgcgcatcaatcgcaatcgttgtcaatattgccgcctgaaaaagtgcattgccgtgggcatgagtcgcgatgctgtgcgttttggacgcgtgccgaagcgcgaaaaggcgcgtatcctggcggccatgcaacagagcacccagaatcgcggccagcagcgagccctcgccaccgagctggatgaccagccacgcctcctcgccgccgtgctgcgcgcccacctcgagacctgtgagttcaccaaggagaaggtctcggcgatgcggcagcgggcgcgggattgcccctcctactccatgcccacacttctggcctgtccgctgaaccccgcccctgaactgcaatcggagcaggagttctcgcagcgtttcgcccacgtaattcgcggcgtgatcgactttgccggcatgattcccggcttccagctgctcacccaggacgataagttcacgctcctgaaggcgggactcttcgacgccctgtttgtgcgcctgatctgcatgtttgactcgtcgataaactcaatcatctgtctaaatggccaggtgatgcgacgggatgcgatccagaacggagccaatgcccgcttcctggtggactccaccttcaatttcgcggagcgcatgaactcgatgaacctgacagatgccgagataggcctgttctgcgccatcgttctgattacgccggatcgccccggtttgcgcaacctggagctgatcgagaagatgtactcgcgactcaagggctgcctgcagtacattgtcgcccagaataggcccgatcagcccgagttcctggccaagttgctggagacgatgcccgatctgcgcaccctgagcaccctgcacaccgagaaactggtagttttccgcaccgagcacaaggagctgctgcgccagcagatgtggtccatggaggacggcaacaacagcgatggccagcagaacaagtcgccctcgggcagctgggcggatgccatggacgtggaggcggccaagagtccgcttggctcggtatcgagcactgagtccgccgacctggactacggcagtccgagcagttcgcagccacagggcgtgtctctgccctcgccgcctcagcaacagccctcggctctggccagctcggctcctctgctggcggccaccctctccggaggatgtcccctgcgcaaccgggccaattccggctccagcggtgactccggagcagctgagatggatatcgttggctcgcacgcacatctcacccagaacgggctgacaatcacgccgattgtgcgacaccagcagcagcaacaacagcagcagcagatcggaatactcaataatgcgcattcccgcaacttgaatgggggacacgcgatgtgccagcaacagcagcagcacccacaactgcaccaccacttgacagccggagctgcccgctacagaaagctagattcgcccacggattcgggcattgagtcgggcaacgagaagaacgagtgcaaggcggtgagttcggggggaagttcctcgtgctccagtccgcgttccagtgtggatgatgcgctggactgcagcgatgccgccgccaatcacaatcaggtggtgcagcatccgcagctgagtgtggtgtccgtgtcaccagttcgctcgccccagccctccaccagcagccatctgaagcgacagattgtggaggatatgcccgtgctgaagcgcgtgctgcaggctccccctctgtacgataccaactcgctgatggacgaggcctacaagccgcacaagaaattccgggccctgcggcatcgcgagttcgagaccgccgaggcggatgccagcagttccacttccggctcgaacagcctgagtgccggcagtccgcggcagagcccagtcccgaacagtgtggccacgcccccgccatcggcggccagcgccgccgcaggtaatcccgcccagagccagctgcacatgcacctgacccgcagcagccccaaggcctcgatggccagctcgcactcggtgctggccaagtctctcatggccgagccgcgcatgacgcccgagcagatgaagcgcagcgatattatccaaaactacttgaagcgcgagaacagcacagcagccagcagcaccaccaatggcgtgggcaaccgcagtcccagcagcagctccacaccgccgccgtcggcggtccagaatcagcagcgttggggcagcagctcggtgatcaccaccacctgccagcagcgccagcagtccgtgtcgccgcacagcaacggttccagctccagttcgagctctagctccagctccagttcgtcatcctcctccacatcctccaactgcagctccagctcggccagcagctgccagtatttccagtcgccgcactccaccagcaacggcaccagtgcaccggcgagctccagttcgggatcgaacagcgccacgcccctgctggaactgcaggtggacattgctgactcggcgcagcctctcaatttgtccaagaaatcgcccacgccgccgcccagcaagctgcacgctctggtggccgccgccaatgccgttcaaaggtatcccacattgtccgccgacgtcacagtgacagcctccaatggcgggtcctccgtcggcggcggcgagtccggccgccagcagcagtccgccggcgagtgtgggctcccccaatccgggcctgagcgccgccgtgcacaaggtaatgctggaggcgtaagagcgggaggaggtaggtggttttacgcggagaagtgggagagacagagactgggagtggcagttcagcgaagcaggaagcaggatcacttggagcggcgggagttgaattaaattattttaccatttaattgagacgtgtacaaagtttgaaagcaaaaccaacatgcatgcaatttaaaactaatatttaaagcaacaacaaacaaaacaactacaagttattaatttaaaaaacaaacaaacaaacaaacaacaaaaaacccaagcttgaatggtattacaaaagaaaaagaaaaacagaaaaaatataaatatattttagcagttaaactttaacgtagcaagaaaccaacaaacccaaggcagcgctctgatttcgcattaacttttcttcagctgctaccgaaaacgcccctcacctcccccccacccaacccttcctccacacaccaaccgtctttcgacccctgattgttttataagttttaagctcttgttgtacatattaattacgtttattggtaactatgtttagcgctttagttgtagttggagcaaaactactttgcttttttggatgttttttgaaaaaactgcaaattattattattaaatttttaaatacctaaaaacaaaacaatgtgtgtgaaattttttattgtgcgatctccaagcagaatgaagtgcagtttgcaacaaattttaactacgattaagttgataacgattcattttttatgaatttaactaattttatgaatttgttatagttttccacccttctatagatctttctatctgatcatctagctacccgtattcctgatttctcctttggcacaaagctcttctctatgctaaagaatcaagtggaataaatattgttttctaattttaaaactaccacaaaaatacgattaaaatatacacgaagtaattgaaaatcaaacaaaatgcttaaagttttagcagcaagcagtaaaacgacgatgaagaagagaaacccaacgttaaatatatctgttgtgtacatagttaaatgttaaattaaacacaaaaacatatttaaagtacatataaatacacataattattaatgaagaaacctatgcttaaaagattcaatgtttgattggcatcttagaaaaccaagcgaaaaatacaaaaaaaaatcaacaaacaaaaattatgatatattatttaaaagtaaagtatacatttacattacagaaaaacaaaagagaaaacttgcggtagcaacaaaactattatattaattacattttaattatgctgtactattatgattattaattattatgattaattaattacgatttttatgcttagacaaaccaacaaaaaacaaatatgcaaaaaccattaaaaaaaaaaacaaaaaacaagcaaaaaattacactggcgcagaaatttgtattgtcaaatcaagaaaaaatttgtttaaaaatttcaaaagaaagcctcttaagttttttcatttcaatttccttttcagttgaacactttttcttaaaatgttcagttttaccgttactttgctttggagtggtaagattttgggtttcacttgatttcattttcgttttgactatcgtgctgtaaaaatatttactaaattatatcgaaagtatttatatcataattaaataaacaaga

SEQ ID NO: 34 - protein, 1237 aa, Drosophila melanogaster
MLMSADSSDSAKTSVICSTVSASMLAPPAPEQPSTTAPPILGVTGRSHLENALKLPPNTSVSAYYQHNSKLGMGQNYNPEFRSLVAPVTDLDTVPPTGVTMASSSNSPNSSVKLPHSGVIFVSKSSAVSTTDGPTAVLQQQQPQQQMPQHFESLPHHHPQQEHQPQQQQQQHHLQHHPHPHVMYPHGYQQANLHHSGGIAVVPADSRPQTPEYIKSYPVMDTTVASSVKGEPELNIEFDGTTVLCRVCGDKASGFHYGVHSCEGCKGFFRRSIQQKIQYRPCTKNQQCSILRINRNRCQYCRLKKCIAVGMSRDAVRFGRVPKREKARILAAMQQSTQNRGQQRALATELDDQPRLLAAVLRAHLETCEFTKEKVSAMRQRARDCPSYSMPTLLACPLNPAPELQSEQEFSQRFAHVIRGVIDFAGMIPGFQLLTQDDKFTLLKAGLFDALFVRLICMFDSSINSIICLNGQVMRRDAIQNGANARFLVDSTFNFAERMNSMNLTDAEIGLFCAIVLITPDRPGLRNLELIEKMYSRLKGCLQYIVAQNRPDQPEFLAKLLETMPDLRTLSTLHTEKLVVFRTEHKELLRQQMWSMEDGNNSDGQQNKSPSGSWADAMDVEAAKSPLGSVSSTESADLDYGSPSSSQPQGVSLPSPPQQQPSALASSAPLLAATLSGGCPLRNRANSGSSGDSGAAEMDIVGSHAHLTQNGLTITPIVRHQQQQQQQQQIGILNNAHSRNLNGGHAMCQQQQQHPQLHHHLTAGAARYRKLDSPTDSGIESGNEKNECKAVSSGGSSSCSSPRSSVDDALDCSDAAANHNQVVQHPQLSVVSVSPVRSPQPSTSSHLKRQIVEDMPVLKRVLQAPPLYDTNSLMDEAYKPHKKFRALRHREFETAEADASSSTSGSNSLSAGSPRQSPVPNSVATPPPSAASAAAGNPAQSQLHMHLTRSSPKASMASSHSVLAKSLMAEPRMTPEQMKRSDIIQNYLKRENSTAASSTTNGVGNRSPSSSSTPPPSAVQNQQRWGSSSVITTTCQQRQQSVSPHSNGSSSSSSSSSSSSSSSSSTSSNCSSSSASSCQYFQSPHSTSNGTSAPASSSSGSNSATPLLELQVDIADSAQPLNLSKKSPTPPPSKLHALVAAANAVQRYPTLSADVTVTASNGGSSVGGGESGRQQQSAGECGLPQSGPERRRAQGNAGGVRAGGGRWFYAEKWERQRLGVAVQRSRKQDHLERRELN

SEQ ID NO: 35 - DNA, 25 bp, Drosophila melanogaster
  misc_feature (1)..(25): n is a, c, g, or t
cgggatcctgyaarggnttyttyaa

SEQ ID NO: 36 - DNA, 25 bp, Drosophila melanogaster
  misc_feature (1)..(25): y is t/u or g
cgggatcctgycayathgayaarac

SEQ ID NO: 37 - DNA, 30 bp, Drosophila melanogaster
  misc_feature (1)..(30): n is a, c, g, or t
aargtngaygaycaratgaaraagcttggg

SEQ ID NO: 38 - DNA, 27 bp, Drosophila melanogaster
  misc_feature (1)..(27): n is a, c, g, or t
aayacngtnttyttyaaraagcttggg

SEQ ID NO: 39 - DNA, 27 bp, Heliothis virescens
aatgtctgcactgcgatttgtcgctgt

SEQ ID NO: 40 - DNA, 28 bp, Heliothis virescens
acagatccctcaagtgtcgtctctcacg

SEQ ID NO: 41 - DNA, 27 bp, Heliothis virescens
attgtaagtctggctctgcaagagccc

SEQ ID NO: 42 - DNA, 28 bp, Heliothis virescens
cgtgagagacgacacttgagggatctgt

SEQ ID NO: 43 - protein, 462 aa, Bombyx mori
MSSVAKKDKRTMSVTALINRAWPMTPSPQQQQQMVPSTQHSNFLHAMATPSTTPNVELDIQWLNIESGFMSPMSPPEMKPDTAMLDGFRDDSTPPPPFKNYPPNHPLSGSKHLCSICGDRASGKHYGVYSCEGCKGFFKRTVRKDLTYACREDKNCIIDKRQRNRCQYCRYQKCLACGMKREAVQEERQRAARRTEDAHPSSSVQELSIERLLELEALVADSAEELQILRVGPESGVPAKYRAPVSSLCQIGNKQIAALIVWARDIPHFGQLEIDDQILLIKGSWNELLLFAIAWRSMEFLNDERENVDSRNTAPPQLICLMPGMTLHRNSALQAGVGQIFDRVLSELSLKMRSLRMDQAECVALKAIILLNPDVKGLKNKQEVDVLREKMFLCLDEYCRRSRGGEEGRFAALLLRLPALRSISLKSFEHLYLFHLVAEGSVSSYIRDALCNHAPPIDTNIM

SEQ ID NO: 44 - protein, 461 aa, Manduca sexta
MSSVAKKDKRTMSVTALINRAWPLTPAPHQQQSMPSSQPSNFLQPLATPSTTPSVELDIQWLNIEPGFMSPMSPPEMKPDTAMLDGLRDDSTPPPAFKNYPPNHPLSGSKHLCSICGDRASGKHYGVYSCEGCKGFFKRTVRKDLTYACREDRNCIIDKRQRNRCQYCRYQKCLACGMKREAVQEERQRAARGTEDAHPSSSVQELSIERLLEIESLVADPPEEFQFLRVGPESGVPAKYRAPVSSLCQIGNKQIAALVVWARDIPHFGQLELEDQILLIKNSWNELLLFAIAWRSMEYLTDERENVDSRSTAPPQLMCLMPGMTLHRNSALQAGVGQIFDRVLSELSLKMRTLRMDQAEYVALKAIILLNPDVKGLKNKPEVVVLREKMFSCLDEYVRRSRCAEEGRFAALLLRLPALRSISLKCFEHLYFFHLVADTSIASYIHDALRNHAPSIDTSIL

SEQ ID NO: 45 - protein, 472 aa, Choristoneura fumiferana
MSSVAKKDKPTMSVTALINWARPAPPGPPQPQSASPAPAAMLQQLPTQSMQSLNHIPTVDCSLDMQWLNLEPGFMSPMSPPEMKPDTAMLDGLRDDATSPPNFKNYPPNHPLSGSKHLCSICGDRASGKHYGVYSCEGCKGFFKRTVRKDLSYACREERNCIIDKRQRNRCQYCRYQKCLACGMKREAVQEERQRNARGAEDAHPSSSVQVSDELSIERLTEMESLVADPSEEFQFLRVGPDSNVPPRYRAPVSSLCQIGNKQIAALVVWARDIPHFGQLELDDQVVLIKASWNELLLFAIAWRSMEYLEDERENGDGTRSTTQPQLMCLMPGMTLHRNSAQQAGVGAIFDRVLSELSLKMRTLRMDQAEYVALKAIVLLNPDVKGLKNRQEVDVLREKMFSCLDDYCRRSRSNEEGRFASLLLRLPALRSISLKSFEHLYFFHLVAEGSISGYIREALRNHAPPIDVNAMM

SEQ ID NO: 46 - protein, 389 aa, Locusta migratoria
MEGSERGISLENNLSISSMGPQSPLDMKPDTASLISSGSFSPTGGPNSPGSFTIGHSSLLNNSSSNQAKGSSSQYPPNHPLSGSKHLCSICGDRASGKHYGVYSCEGCKGFFKRTVRKDLSYACREDKNCIIDKRQRNRCQYCRYQKCLAMGMKREAVQEERQRTKERDQNEVESTSSLHTDMPVERILEAEKRVECKAENQVEYELVEWAKHIPHFTSLPLEDQVLLLRAGWNELLIAAFSHRSVDVKDGIVLATGLTVHRNSAHQAGVGTIFDRVLTELVAKMREMKMDKTELGCLRSVILFNPEVRGLKSAQEVELLREKVYAALEEYTRTTHPDEPGRFAKLLLRLPSLRSIGLKCLEHLFFFRLIGDVPIDTFLMEMLESPSDS

SEQ ID NO: 47 - protein, 552 aa, Chironomus tentans
MLKKEKPMMTVAAIIEQAQNRWMDHPLVYNSRSLQFQGSYCIDSSLLGHMGPLSPPDLKPDISLLNCNNNNNNTNNNNSNSSHNNLNHHNTSPLPVLGANTFSPIQSLNNNGPSSPLSSIGNGSGTIVTFNQIKLQSPSPSNASSSSTLSGPLTTTPPATNANNILGMGNGNCGNTANGKQSQYPPNHPLSGSKHLCSICGDRASGKHYGVYSCEGCKGFFKRTVRKDLSYACREERNCVIDKKQRNRCQYCRYQKCLNCGMKREAVQEERQRGGKSQKGDDMSISSTQSLVNNGPGRDITVERLMEADQMSEARCGDKSIQYLRVAASNTMIPPEYRAPVSAICAMVNKQVFQHMDFCRRLPHFTKLPLNDQMYLLKQSLNELLILNIAYMSIQYVEPDRRNADGSLERRQISQQMCLSRNYTLGRNMAVQAGVVQIFDRILSELSVKMKRLDLDATELCLLKSIVVFNPDVRTLDDRKSIDLLRSRIYASLDEYCRQKHPNEDGRFAQLLLRLPALRSISLKCLDHLFYFQLIDDKNVENSVIEEFHKLN

SEQ ID NO: 48 - protein, 508 aa, Drosophila melanogaster
MDNCDQDASFRLSHIKEEVKPDISQLNDSNNSSFSPKAESPVPFMQAMSMVHVLPGSNSASSNNNSAGDAQMAQAPNSAGGSAAAAVQQQYPPNHPLSGSKHLCSICGDRASGKHYGVYSCEGCKGFFKRTVRKDLTYACRENRNCIIDKRQRNRCQYCRYQKCLTCGMKREAVQEERQRGARNAAGRLSASGGGSSGPGSVGGSSSQGGGGGGGVSGGMGSGNGSDDFMTNSVSRDFSIERIIEAEQRAETQCGDRALTFLRVGPYSTVQPDYKGAVSALCQVVNKQLFQMVEYARMMPHFAQVPLDDQVILLKAAWIELLIANVAWCSIVSLDDGGAGGGGGGLGHDGSFERRSPGLQPQQLFLNQSFSYHRNSAIKAGVSAIFDRILSELSVKMKRLNLDRRELSCLKAIILYNPDIRGIKSRAEIEMCREKVYACLDEHCRLEHPGDDGRFAQLLLRLPALRSISLKCQDHLFLFRITSDRPLEELFLEQLEAPPPPGLAMKLE

SEQ ID NO: 49 - protein, 555 aa, Bombyx mori
MHEDAPKMSIAQSLAASTSQPKGDIVTEIPLEFAMSSMETKSIETTNVELKITYVDPTTGTGGEPGAYLPTAGTVCDQTDTKDVIEELCPVCGDKVSGYHYGLLTCESCKGFFKRTVQNKKVYTCVAERACHIDKTQRKRCPFCRFQKCLDVGMKLEAVRADRMRGGRNKFGPMYKRDRARKLQMMRQRQIAVQTLRGSLGDGGLVLGFGSPYTAVSVKQEIQIPQVSSLTSSPESSPGPALLRAQPQPPQPPPPPTHDKWEAHSPHSASPDAFTFDTQSNTAATPSSTAEATSTETLRVSPMIREFVQTVDDREWQNALFGLLQSQTYNQCEVDLFELMCKVLDQNLFSQVDWARNTVFFKYLKVDDQMKLLQDSWSVMLVLDHLHQRMHNGLPDETTLHNGQKFDLLCLGLLGVPSLADHFNELQNKLAELKFDVPDYICVKFMLLLNPEVRGIVNVKCVREGYQTVQAALLDYPYLLSTIQDKFGKLVMVVPEIHALRLGEKSTCTSGIVQARHLPRLFSWKCCTQNANLEVPVTNKVEELRSAKPRRHHNK

SEQ ID NO: 50 - protein, 699 aa, Manduca sexta
MTLVMSPDSSYGRYDAPTPTDVTSPVHREREPELHIEFDGTTVLCRVCGDKASGFHYGVHSCEGCKGFFRRSIQQKIQYRPCTKNQQCSILRINRNRCQYCRLKKCIAVGMSRDAVRFGRVPKREKARILAAMQQSSTSRAHEQAAAAELDDAPRLLARVVRAHLDTCEFTRDRVAAMRARARDCPTYSQPTLACPLNPAPELQSEKEFSQRFAHVIRGVIDFAGLIPGFQLLTQDDKFTLLKSGLFDALFVRLICMFDAPLNSIICLNGQLMKRDSIQSGANARFLVDSTFKFAERMNSMNLTDAEIGLFCAIVLITPDRPGLRNVELVERMHTRLKACLQTVIAQNRPDRPGFLRELMDTLPDLRTLSTLHTEKLVVFRTEHKELLRQQMWSEEEAVSWVDSGADELARSPIGSVSSSESGEAVGDCGTPLLAATLAGRRRLDSRGSVDEEALGVAHLAHNGLTVTPVRQPPRYRKLDSPTDSGIESGNEKHERIVGTGSGCSSPRSSLEEHNEDRRPPVSADDMPVLKRVLQAPPLYGGTPSLMDEAYRRHKKFRALRRDTGEAEARTVRPTPSPQPQHPHPANPAHPAHSPRPQRASLSSTHSVLAKSLMEGPRMTPEQLKRTDIIQQYMRRGESSAPAEGCPLRAGGLLTCYRGASPAPQPVLALQVDVTDAPLNLSKKSPSPPRTYMPQMLEA

SEQ ID NO: 51 - protein, 1394 aa, Drosophila melanogaster
MVCAMQEVAAVQHQQQQQQLQLPQQQQQQQQTTQQQHATTIVLLTGNGGGNLHIVATPQQHQPMHQLHHQHQHQHQHQQQAKSQQLKQQHSALVKLLESAPIKQQQQTPKQIVYLQQQQQQPQRKRLKNEAAIVQQQQQTPATLVKTTTTSNSNSNNTQTTNSISQQQQQHQIVLQHQQPAAAATPKPCADLSAKNDSESGIDEDCPNSDEDCPNANPAGTSLEDSSYEQYQCPWKKIRYARELLKQRELEQQQTTGGSNAQQQVEAKPAAIPTSNIKQLHCDSPFSAQTHKEIANLLRQQSQQQQVVATQQQQQQQQQHQHQQQRRDSSDSNCSLMSNSSNSSAGNCCTCNAGDDQQLEEMDEAHDSGCDDELCEQHHQRLDSSQLNYLCQKFDEKLDTALSNSSANTGRNTPAVTANEDADGFFRRSIQQKIQYRPCTKNQQCSILRINRNRCQYCRLKKCIAVGMSRDAVRFGRVPKREKARILAAMQQSTQNRGQQRALATELDDQPRLLAAVLRAHLETCEFTKEKVSAMRQRARDCPSYSMPTLLACPLNPAPELQSEQEFSQRFAHVIRGVIDFAGMIPGFQLLTQDDKFTLLKAGLFDALFVRLICMFDSSINSIICLNGQVMRRDAIQNGANARFLVDSTFNFAERMNSMNLTDAEIGLFCAIVLITPDRPGLRNLELIEKMYSRLKGCLQYIVAQNRPDQPEFLAKLLETMPDLRTLSTLHTEKLVVFRTEHKELLRQQMWSMEDGNNSDGQQNKSPSGSWADAMDVEAAKSPLGSVSSTESADLDYGSPSSSQPQGVSLPSPPQQQPSALASSAPLLAATLSGGCPLRNRANSGSSGDSGAAEMDIVGSHAHLTQNGLTITPIVRHQQQQQQQQQIGILNNAHSRNLNGGHAMCQQQQQHPQLHHHLTAGAARYRKLDSPTDSGIESGNEKNECKAVSSGGSSSCSSPRSSVDDALDCSDAAANHNQVVQHPQLSVVSVSPVRSPQPSTSSHLKRQIVEDMPVLKRVLQAPPLYDTNSLMDEAYKPHKKFRALRHREFETAEADASSSTSGSNSLSAGSPRQSPVPNSVATPPPSAASAAAGNPAQSQLHMHLTRSSPKASMASSHSVLAKSLMAEPRMTPEQMKRSDIIQNYLKRENSTAASSTTNGVGNRSPSSSSTPPPSAVQNQQRWGSSSVITTTCQQRQQSVSPHSNGSSSSSSSSSSSSSSSSSTSSNCSSSSASSCQYFQSPHSTSNGTSAPASSSSGSNSATPLLELQVDIADSAQPLNLSKKSPTPPPSKLHALVAAANAVQRYPTLSADVTVTASNGGSSVGGGESGRQQQSAGECGLPQSGPERRRAQGNAGGVRAGGGRWFYAEKWERQRLGVAVQRSRKQDHLERRELN

SEQ ID NO: 52 - protein, 685 aa, Manduca sexta
MVRAMSCGAELRERHSVLVSMLEARRESSDSGCSSDDGSDVERDCKCRCDPQGFFRRSIQQKIQYRPCTKNQQCSILRINRNRCQYCRLKKCIAVGMSRDAVRFGRVPKREKARILAAMQQSSTSRAHEQAAAAELDDAPRLLARVVRAHLDTCEFTRDRVAAMRARARDCPTYSQPTLACPLNPAPELQSEKEFSQRFAHVIRGVIDFAGLIPGFQLLTQDDKFTLLKSGLFDALFVRLICMFDAPLNSIICLNGQLMKRDSIQSGANARFLVDSTFKFAERMNSMNLTDAEIGLFCAIVLITPDRPGLRNVELVERMHTRLKACLQTVIAQNRPDRPGFLRELMDTLPDLRTLSTLHTEKLVVFRTEHKELLRQQMWSEEEAVSWVDSGADELARSPIGSVSSSESGEAVGDCGTPLLAATLAGRRRLDSRGSVDEEALGVAHLAHNGLTVTPVRQPPRYRKLDSPTDSGIESGNEKHERIVGTGSGCSSPRSSLEEHNEDRRPPVSADDMPVLKRVLQAPPLYGGTPSLMDEAYRRHKKFRALRRDTGEAEARTVRPTPSPQPQHPHPANPAHPAHSPRPQRASLSSTHSVLAKSLMEGPRMTPEQLKRTDIIQQYMRRGESSAPAEGCPLRAGGLLTCYRGASPAPQPVLALQVDVTDAPLNLSKKSPSPPRTYMPQMLEA

SEQ ID NO: 53 - protein, 1443 aa, Drosophila melanogaster
MHGGGPGSSGSNIIRRSSGSFPGSGSGSASKLIKTEPIDFEMLHLEENERQQDIEREPSSSNSNSNSNSLTPQRYTHVQVQTVPPRQPTGLTTPGGTQKVILTPRVEYVQQRATSSTGGGMKHVYSQQQGTAASRSAPPETTALLTTTSGTPQIIITRTLPSNQHLSRRHSASPSALHHYQQQQQPQRQQSPPPLHHQQQQQQQHVRVIRDGRLYDEATVVVAARRHSVSPPPLHHHSRSAPVSPVIARRGGAAAYMDQQYQQRQTPPLAPPPPPPPPPPPPPPPQQQQQQYISTGVPPPTAAARKFVVSTSTRHVNVIASNHFQQQQQQHQAQQHQQQHQQHVIASVSSSSSSSAIGSGGSSSSHIFRTPVVSSSSSSNMHHQQQQQQQQSSLGNSVMRPPPPPPPPKVKHASSSSSGNSSSSNTNNSSSSSNGEEPSSSIPDLEFDGTTVLCRVCGDKASGFHYGVHSCEGCKGFFRRSIQQKIQYRPCTKNQQCSILRINRNRCQYCRLKKCIAVGMSRDAVRFGRVPKREKARIWRPCNRAPRIAASSDPSPPSWMTSHASSPPCCCAHLETCEFTKEKVSAMRHGRGLPSTPCHTSGLSAEPAPELQSEQEFSQRFAHVIRGVIDFAGMIPGFQLLTQDDKFTLLKAGLFDALFVRLICMFDSSINSIICLNGQVMRRDAIQNGANARFLVDSTFNFAERMNSMNLTDAEIGLFCAIVLITPDRPGLRNLELIEKMYSRLKGCLQYIVAQNRPDQPEFLAKLLETMPDLRTLSTLHTEKLVVFRTEHKELLRQQMWSMEDGNNSDGQQNKSPSGSWADAMDVEAAKSPLGSVSSTESADLDYGSPSSSQPQGVSLPSPPQQQPSALASSAPLLAATLSGGCPLRNRANSGSSGDSGAAEMDIVGSHAHLTQNGLTITPIVRHQQQQQQQQQIGILNNAHSRNLNGGHAMCQQQQQHPQLHHHLTAGAARYRKLDSPTDSGIESGNEKNECKAVSSGGSSSCSSPRSSVDDALDCSDAAANHNQVVQHPQLSVVSVSPVRSPQPSTSSHLKRQIVEDMPVLKRVLQAPPLYDTNSLMDEAYKPHKKFRALRHREFETAEADASSSTSGSNSLSAGSPRQSPVPNSVATPPPVAASAAAGNPAQSQLHMHLTRSSPKASMASSHSVLAKSLMAEPRMTPEQMKRSDIIQNYLKRENSTAASSTTNGLGNRSPSSSSTPPPSVQNQQRWGSSSVITTTCQQRQQSVSPHSNGSSSSSSSSSSSSSSSSSTSSNCSSSSASSCQYFQSPHSTSIGTGEPDGAPVRDRTAPRPCWNCRWTLLTRRTSQFVQEIAHAAAQQAARSGGRRQCRSKVSHIVRRRHSDSLQWRSSVGGGESGAQQQSAGECGLPQSGPERRRAQGNAGGVRAGGGRWFYAEKWERQRLGVAVQRSRKQDHLERRELN

SEQ ID NO: 54 - protein, 690 aa, Choristoneura fumiferana
MTLVMSPDSSYGRYDAQPPVDGGMVNPVHREREPELHIEFDGTTVLCRVCGDKASGFHYGVHSCEGCKGFFRRSIQQKIQYRPCTKNQQCSILRINRNRCQYCRLKKCIAVGMSRDAVRFGRVPKREKARILAAMQQSSSSRAHEQAAAAELDDAPRLLARVVRAHLDTCEFTRDRVAAMRARARDCPTYSQPTLACPLNPAPELQSEKEFSQRFAHVIRGVIDFAGLIPGFQLLTQDDKFTLLKSGLFDALFVRLICMFDAPLNSIICLNGQLMKRDSIQSGANARFLVDSTFKFAERMNSMNLTDAEIGLFCAIVLITPDRPGLRNIELVERMHARLKSCLQTVIAQNRADRPGFLRELMDTLPDLRTLSTLHTEKLVVFRTEHKELLRQQMWGDEEVCPWADSGVDDSARSPLGSVSSSESGEAPSDCGTPLLAATLAGRRRLDSRGSVDEEALGVAHLAHNGLTVTPVRPPPRYRKLDSPTDSGIESGNEKHERIVGPGSGCSSPRSSLEEHMEDRRPLAADDMPVLKRVLQAPPLYDASSLMDEAYKPHKKFRAMRRDTGEAEARPMRPTPSPQPMHPHPGSPAHPAHPAHSPRPLRAPLSSTHSVLAKSLMEGPRMTPEQLKRTDIIQQYMRRGEAGEECRAGLLLYRGASPLQVDVADAPQPLNLSKKSPSPPRSFMPPMLEA

SEQ ID NO: 55 - protein, 711 aa, Galleria mellonella
MTLVMSPDSSYGRYDAPAPADNRIMSPVHKEREPELHIEFDGTTVLCRVCGDKASGFHYGVHSCEGCKGFFRRSIQQKIQYRPCTKNQQCSILRINRNRCQYCRLKKCIAVGMSRDAVRFGRVPKREKARILAAMQSSTTRAHEQAAAAELDDGPRLLARVVRAHLDTCEFTRDRVAAMRNGARDCPTYSQPTLACPLNPAPELQSEKEFSQRFAHVIRGVIDFAGLIPGFQLLTQDDKFTLLKSGLFDALFVRLICMFDAPLNSIICLNGQLMKRDSIQSGANARFLVDSTFKFAERMNSMNLTDAEIGLFCAIVLITPDRPGLRNVELVERMHSRLKSCLQTVIAQNRSDGPGFLRELMDTLPDLRTLSTLHTEKLVVFRTEHKELLRQQMWVEDEGALWADSGADDSARSPIGSVSSSESSETTGDCGTPLLAATLAGRRRLDSRGSVDEEALGVAHLAHNGLTVTPVRPPPRYRKLDSPTDSGIESGNEKHERIVGPESGCSSPRSSLEEHSDDRRPIAPADDMPVLKRVLQAPPLYDASSLMDEAYKPHKKFRAMRRDTWSEAEARPGRPTPSPQPPHHPHPASPAHPAHSPRPIRAPLSSTHSVLAKSLMEGPRMTPEQLKRTDIIQQYMRRGETGAPTEGCPLRAGGLLTCFRGASPAPQPVIALQVDVAETDAPQPLNLSKKSPSPSPPPPPPRSYMPPMLPA

SEQ ID NO: 56 - protein, 711 aa, Metapenaeus ensis; identical to SEQ ID NO: 55

(19) United States
(12) Patent Application Publication
(10) Pub. No.: US 2005/0176928 A1
(43) Pub. Date: Aug. 11, 2005

(54) Insect nuclear receptor genes and uses thereof

(75) Inventors: Julie A Broadus, Bethesda, MD (US); Blanche Covington Brown, Chapel Hill, NC (US); Lynn F. Stam, Raleigh, NC (US); Kim P. Kamdar, Boston, MA (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 10/467,555

(22) PCT Filed: Mar. 22, 2002
(86) PCT No.: PCT/US02/11257

Related U.S. Application Data
(60) Provisional application No. 60/278,336, filed on Mar. 23, 2001.

(51) Int. Cl. C07K 14/705; C07H 21/04; C12N 5/06

(52) U.S. Cl. 530/350; 435/69.1; 435/320.1; 435/348; 536/23.5

(57) ABSTRACT

The present invention provides isolated nucleic acids encoding insect nuclear receptor polypeptides, isolated insect nuclear polypeptides, and uses thereof. The disclosed insect nuclear receptor nucleic acids and polypepides can be used in screening assays to identify insecticidal compounds. The disclosed insect nuclear receptor nucleic acids and polypeptides can further be used as components of a chimeric expression cassette for inducible gene expression.

### FIELD OF THE INVENTION

The present invention generally relates to nuclear receptor genes. More particularly, the present invention provides novel nuclear receptor nucleic acid and polypeptide sequences, chimeric genes comprising the disclosed nuclear receptor sequences, antibodies that specifically recognize the disclosed nuclear receptor polypeptides, modulators of nuclear receptor nucleic acids and polypeptides, and uses thereof.

Table of Abbreviations

| | |
|---|---|
| ATCC | American Tissue Culture Collection |
| βFTZ-F1 | fushi tarazu transcription factor 1, β |
| | isoform |
| CDS | coding sequence |
| DBD | DNA-binding domain |
| DERR | Drosophila estrogen-related receptor |
| DFAX1 | Drosophila fax-related gene 1 |
| DFAX2 | Drosophila fax-related gene 2 |
| DHR38 | Drosophila hormone receptor 38 |
| DHR39 | Drosophila hormone receptor 39 |
| DHR4 | Drosophila hormone receptor 4 |
| DSF | dissatisfaction |
| dsRNA | double-stranded RNA |
| dsRNAi | double-stranded RNA interference |
| E75A | ecdysone-inducible gene E75, A isoform |
| EcR | ecdysone receptor |
| EGON | eagle nuclear receptor |
| DHR3 | Drosophila hormone receptor 3 |
| DHR78 | Drosophila hormone receptor 78 |
| DHR96 | Drosophila hormone receptor 96 |
| E78 | Drosophila ecdysone-inducible protein 78 |
| EGON | eagle |
| FCS | Fluorescence Correlation Spectroscopy |
| GST | glutathione S transferase |
| HMM | Hidden Markov Model |
| DHNF4 | Drosophila hepatic nuclear factor 4 |
| HR3 | hormone receptor 3 |
| hs | Drosophila hsp70 promoter |
| IPM | integrated pest management |
| JH | juvenile hormone |
| LBD | ligand-binding domain |
| PCR | polymerase chain reaction |
| PEG | polyethylene glycol |
| PTTH | prothoracicotropic hormone |
| RACE | rapid amplification of cDNA ends |
| SELDI-TOF MS | Surface-Enhanced Laser Desorption/ |
| | Ionization Time-of-Flight Mass |
| | Spectroscopy |
| Sf9 cells | Spodoptera frugiperda cells |
| SPR | Surface Plasmon Resonance |
| USP | ultraspiracle |

### BACKGROUND ART

Insects contribute or cause many human and animal diseases, and are responsible for substantial agricultural and property damage. The societal costs associated with insect pests in dollars, time, and suffering are monumental. To combat these problems, insecticidal compounds have been developed and employed. The total worldwide market size for insecticide crop protection is over $5 billion, and insecticide products comprise approximately 32% of world consumption of pesticides.

Insecticide development has been guided predominantly by leadfinding efforts for new chemical structures. According to this strategy, chemical derivatization of a known insecticide is performed, and the synthesized compounds are analyzed for insecticidal activity. An alternative approach relies on methods for detecting molecular interactions between a candidate compound and a target molecule. An ideal target molecule is precisely regulated during insect development, such that modulation of the activity or level of activity of the target molecule results in organismal lethality. High throughput screening methods have enabled rapid screening of diverse and populous compound libraries for an ability to interact with a target molecule. The novel modulators discovered by such methods are useful as insecticides.

A target molecule can be further selected based on modulation of the target molecule activity that results in lethality during larval development. The insect life cycle requires successive larval or nymph stages that are devoted to growth such that the animal can increase mass by several thousand-fold. To sustain this growth, immature insects feed unabated for prolonged periods, and thus are particularly deleterious to agricultural crops during this developmental stage.

Each larval instar concludes with molting of the larval cuticle to accommodate the changing size of the larvae. The apolysis of the old cuticle and the synthesis of new cuticle are regulated by the coordinate action of juvenile hormone and ecdysone (20-hydroxyecdysone, hereafter referred to as ecdysone). The neuropeptide PTTH directs a transient rise in hemolymph titer of ecdysone, which is the trigger for molting. A concomitant high level of juvenile hormone signals resynthesis of larval cuticle, while low juvenile hormone levels signal the synthesis of pupal cuticle and commitment to metamorphosis. See Nijhout (1994) Insect Hormones, Princeton University Press, Princeton, N.J.

Classical endocrinology studies demonstrated that a premature rise in hemolymph ecdysone achieved by feeding larvae ecdysone was sufficient to trigger premature molting. More recently, experiments in Drosophila have provided genetic evidence for ecdysone control of larval molting. Mutation of the ultraspiracle (usp) gene, which encodes the heterodimeric partner of EcR to form the functional ecdysone receptor, results in larval lethality with supernumerary spiracles indicative of an incomplete molt (Perrimon et al. (1985) Genetics 11:23-41; Oro et al. (1992) Development 115(2):449-462). The ecdysoneless gene encodes a protein required for ecdysone synthesis, and animals carrying mutations in ecdysoneless show defective larval molting and death (Henrich et al. (1987) Dev Biol 120(1):50-55; Henrich et al. (1993) Dev Genet 14(5):469-477). Similarly, mutations of the dare and dre4 genes, which encode proteins required for ecdysone synthesis, result in defective molting (Freeman et al. (1999) Development 126:4591-4602; Sliter & Gilbert (1992) Genetics 130:555-568). These studies demonstrate that misregulated ecdysone signaling—either premature or absent—disrupts larval molting and leads to larval death.

Molecular cloning of the ecdysone receptor has provided a foundation for understanding the mechanism of ecdysone signaling and the mode of action for growth regulatory insecticides. The functional ecdysone receptor is a heterodimer of the ecdysone receptor (EcR) and ultraspiracle (USP) nuclear receptor proteins (Koelle et al. (1991) Cell 67:59-77; Koelle (1992) Ph.D. Thesis, Stanford University, Stanford, California; Yao (1993) Cell 71:63-72; Thomas et al. (1993) Nature 362:471-475). Ecdysone enters cells by virtue of its hydrophobic structure, and binds the EcR/USP heterodimer. The hormone-bound or activated receptor binds DNA at regulatory sequences called response elements and therein directs gene transcription. A subset of the immediate gene targets of the ecdysone signal, the primary response genes, are transcription factors that turn on a large group of secondary response genes that ultimately lead to altered cell functions. Thus, the hormone signal elicits a transcription cascade that both amplifies and diversifies the initial signal.

Several insecticides are known to elicit insect lethality by Interfering with nuclear receptor signaling. The non-steroidal ecdysone agonists RH5849 (Wing (1988) Science 241:467-469), RH2485 (methoxyfenozide, Dhadialla et al. (1998) Annu Rev Entom 43: 545-569), and RH5992 (tebufenozide, also known as the insecticide MIMIC®), are chemical ligands for the ecdysone receptor (EcR). See also, Dhadialla et al. (1998) Annu Rev Entom 43: 545-569, incorporated herein by reference, which describes several insecticides with ecdysteroidal and juvenile hormone activity.

At least seven other nuclear receptor genes are regulated by ecdysone, implicating their participation in larval growth and molting as well. Thummel (1997) BioEssays 19(8):669-672. At a molecular level, these nuclear receptors might function in yet undiscovered hormone signaling pathways. Alternatively or in addition, they might modulate ecdysone signaling by heterodimerization and transregulation. See White et al. (1997) Science 276(5309:114-117; Sutherland et al. (1995) Proc Natl Acad Sci USA 92(17):7966-7970.

Functional analysis of orphan nuclear receptors can be addressed using standard molecular and genetic techniques in Drosophila melanogaster (herein after “Drosophila”). In addition to EcR and USP, fourteen nuclear receptor genes have been identified in Drosophila: knirps, knirps-related (knrl), egon/eagle (eg), seven-up (svp), tailless (tll), hepatic nuclear factor 4 (DHNF4), fushi tarazu factor 1 (FTZ-F1), ecdysone-inducible protein 75 (E75), ecdysone-inducible protein 78 (E78), hormone receptor 3 (DHR3), hormone receptor 38 (DHR38), hormone receptor 39 (DHR39), hormone receptor 78 (DHR78), and hormone receptor 96 (DHR96) (reviewed in Thummel (1995) Cell 83:1-20). All such receptors, with the exception of EcR and USP, are designated orphan nuclear receptors to signify that corresponding endogenous ligands have not been identified. The insect proteins KNIRPS, KNIRPS-RELATED, and EGON lack a discernible ligand binding domain, but are classified as nuclear receptors based on the distinctive sequence of their DNA binding domains (Nauber et al. (1988) Nature 336(6198):489-492; Oro et al. (1988) Nature 336(61989):493-496; Higashijima et al. (1996) Development 122(2):527-536).

There exists a continuing demand for insecticides that show improved efficacy and new modes of action. To this end, the present invention discloses a functional characterization of nuclear receptors during Drosophila larval development. Nuclear receptors that confer larval lethality when misregulated are used in biochemical assays as targets for insecticide development. The present invention also discloses several novel insect nuclear receptor polypeptides and nucleic acid molecules encoding the same that are further useful as components of gene switch technology for inducible gene expression.

### SUMMARY OF THE INVENTION

The present invention discloses isolated insect nuclear receptor polypeptides and isolated nucleic acid molecules encoding the same. Preferably, an isolated insect nuclear receptor polypeptide, or functional portion thereof, comprises a polypeptide encoded by the nucleic acid molecule of any one of SEQ ID NOs:1, 5, 9, 13, 17, 19, 21, 23, and 25; a polypeptide encoded by a nucleic acid molecule that is substantially identical to any one of SEQ ID NOs:1, 5, 9, 13, 17, 19, 21, 23, and 25; a polypeptide having an amino acid sequence of any one of SEQ ID NOs:2, 6, 10, 14, 18, 20, 22, 24, and 26; a polypeptide that is a biological equivalent of any one of SEQ ID NOs:2, 6, 10, 14, 18, 20, 22, 24, and 26; or a polypeptide that is immunologically cross-reactive with an antibody that shows specific binding with a polypeptide comprising some or all amino acids of any one of SEQ ID NOs:2, 6, 10, 14, 18, 20, 22, 24, and 26.

The present invention further teaches chimeric genes having a heterologous promoter that drives expression of a nucleic acid sequence encoding an insect nuclear receptor polypeptide. Preferably, the chimeric gene is carried in a vector and introduced into a host cell so that an insect nuclear receptor polypeptide of the present invention is produced. Preferred host cells include but are not limited to a bacterial cell, an insect cell, and a plant cell.

In another aspect of the Invention, a method is provided for detecting a nucleic acid molecule that encodes an insect nuclear receptor polypeptide. According to the method, a biological sample having nucleic acid material is hybridized under stringent hybridization conditions to an insect nuclear receptor nucleic acid molecule of the present invention. Such hybridization enables a nucleic acid molecule of the biological sample and the insect nuclear receptor nucleic acid molecule to form a detectable duplex structure. Preferably, the insect nuclear receptor nucleic acid molecule includes some or all nucleotides of any one of SEQ ID NOs:1, 5, 9, 13, 17, 19, 21, 23, and 25. The present invention further teaches an antibody that specifically recognizes an insect nuclear receptor polypeptide. Preferably, the antibody recognizes some or all amino acids of any one of SEQ ID NOs:2, 6, 10, 14, 18, 20, 22, 24, and 26. A method for producing an insect nuclear receptor antibody is also disclosed, and the method comprises recombinantly or synthetically producing an insect nuclear receptor polypeptide, or portion thereof, as set forth in any one of SEQ ID NOs:2, 6, 10, 14, 18, 20, 22, 24, and 26; formulating the insect nuclear receptor polypeptide so that it is an effective immunogen; immunizing an animal with the formulated polypeptide to generate an immune response that includes production of insect nuclear receptor antibodies; and collecting blood serum from the immunized animal containing antibodies that specifically recognize an insect nuclear receptor polypeptide. Antibody-producing cells can be optionally fused with an immortal cell line whereby a monoclonal antibody that specifically recognizes an insect nuclear receptor polypeptide can be selected.

A method is also provided for detecting a level of insect nuclear receptor polypeptide using an antibody that recognizes an insect nuclear receptor polypeptide of any of SEQ ID NOs:2, 6, 10, 14, 18, 20, 22, 24, and 26. According to the method, a biological sample is obtained from an experimental subject and a control subject, and an insect nuclear receptor polypeptide is detected in the sample by immunochemical reaction with the insect nuclear receptor antibody. Preferably, the antibody recognizes amino acids of any one of SEQ ID NOs:2, 6, 10, 14, 18, 20, 22, 24, and 26; and is prepared according to a method of the present invention for producing such an antibody.

The present invention further discloses a method for identifying a compound that modulates nuclear receptor function. The method comprises: (a) exposing an isolated insect nuclear receptor polypeptide of any one of SEQ ID NOs:2, 6, 10, 14, 18, 20, 22, 24, 26 to one or more compounds, and (b) assaying binding of a compound to the isolated insect nuclear receptor polypeptide. A compound is selected that demonstrates specific binding to the isolated insect nuclear receptor polypeptide. Preferably, the modulator is a chemical compound, a protein, a peptide, a nucleic acid, or an antibody, and was prepared according to a method disclosed herein.

The present invention also provides a method for identifying an insecticidal compound that modulates nuclear receptor function. The method comprises: (a) isolating an insect nuclear receptor polypeptide of any one of even numbered SEQ ID NOs:2-34, wherein modulation of the insect nuclear receptor polypeptide confers lethality of an insect during a larval stage; (b) exposing the isolated insect nuclear receptor polypeptide to a plurality of substances; (c) assaying binding of a substance to the isolated nuclear receptor polypeptide; and (d) selecting a substance that demonstrates specific binding to the isolated insect nuclear receptor polypeptide. Preferably, the modulator is a chemical compound, a protein, a peptide, a nucleic acid, or an antibody, and was prepared according to a method disclosed herein.

The present invention further provides a method for preventing or treating an insect infestation of a plant, the method comprising: (a) preparing an insecticidal composition that is a modulator of an insect nuclear receptor set forth as any one of even-numbered SEQ ID NOs:2-34; and (b) contacting an effective dose of the insecticidal composition with a plant, whereby an insect infestation of the plant is prevented or abrogated. Preferably, the insecticidal composition comprises a chemical compound, a protein, a peptide, a nucleic acid, or an antibody, and was prepared according to a method disclosed herein. Preferably, the insect infestation is abrogated by lethality of the insect. In one embodiment, the insecticidal composition also displays nematicide activity, such that contacting an effective dose of the insecticidal composition with a plant prevents or abrogates a nematode infestation of the plant.

The present invention further provides a method for preventing or abrogating an insect infestation of a plant, the method comprising: (a) expressing in a plant an insect nuclear receptor modulator that modulates the activity of an insect nuclear receptor polypeptide of any one of even-numbered SEQ ID NOs:2-34, whereby an insect infestation of a plant is prevented or abrogated. Preferably, the insecticidal composition comprises a protein, a peptide, a nucleic acid, or an antibody. In one embodiment, the insecticidal composition additionally displays nematicidal activity, such that expression of insect nuclear receptor modulator in a plant prevents or abrogates a nematode infestation of the plant. The present invention further embodies plants, plant tissues, plant seeds, and plant cells that express an insect nuclear receptor modulator and that are therefore able to inhibit plant parasitic infestation.

The present invention also discloses a chimeric nuclear receptor cassette comprising a DNA binding domain, a ligand binding domain, a hinge domain, and an activation or repression domain, wherein one or more of the DNA binding domain, ligand binding domain, hinge domain, or activation of repression domain is identical or substantially identical to a portion of any one of SEQ ID NOs:2, 6, 10, 14, 18, 20, 22, 24, and 26.

Also disclosed is a method of inducing expression of a target nucleic acid sequence. In a preferred embodiment, the method comprises: (a) constructing a chimeric nuclear receptor expression cassette wherein one or more of the DNA binding domain, ligand binding domain, and activation/repression domains is identical or substantially identical to a portion of any one of SEQ ID NOs:2, 6, 10, 14, 18, 20, 22, 24, and 26; (b) constructing a target expression cassette having a target nucleotide sequence and a cis-regulatory element that is recognized by a DNA binding domain of the chimeric nuclear; (c) expressing the chimeric nuclear receptor expression cassette and the target expression cassette in a heterologous organism; and (d) contacting a ligand that binds to the ligand binding domain of the chimeric nuclear receptor with the organism, whereby the target nucleotide sequence is expressed. In one embodiment, the method is performed to induce gene expression in a plant. The present invention also encompasses plants, plant tissues, plant seeds, and plant cells comprising a disclosed nuclear receptor expression cassette and a target expression cassette.

Accordingly, it is an object of the present invention to provide novel insect nuclear receptor nucleic acids and polypeptides, and novel methods relating thereto. This object is achieved in whole or in part by the present invention.

An object of the invention having been stated above, other objects and advantages of the present invention will become apparent to those skilled in the art after a study of the following description of the invention, Figures, and non-limiting Examples.

### BRIEF DESCRIPTION OF THE DRAWINGS

FIG. 1 is a neighbor-joining tree generated using the pileup feature of the GCG sequence analysis program (Devereux et al. (1984) Nuc Acids Res 12:387-395). The tree depicts relationships among the homeodomain regions of Drosophila nuclear receptors (eagle, GenBank Accession No. D43634; knirps-related, GenBank Accession No. X14153; knirps, GenBank Accession No. X13331; DHR4, GenBank Accession No. AL035245 and SEQ ID NO:14; dissatisfaction, GenBank Accession No. AF106677; tailless, GenBank Accession No. AF019362; FTZ-F1, GenBank Accession No. M98397; DHR39, GenBank Accession No. L07551; E78, GenBank Accession No. U01087; E75, GenBank Accession No. X51548; DHR3, GenBank Accession No. M90806; DHR78, GenBank Accession No. U36791; sevenup, GenBank Accession No. M28863; USP, GenBank Accession No. X53417; DHNF4, GenBank Accession No. U70874; DHR38, GenBank Accession No. X89246; EcR, GenBank Accession No. M74078; DHR96, GenBank Accession No. U36792; DERR, SEQ ID NO:2; DFAX1, SEQ ID NO:6; DFAX2, SEQ ID NO:10).

FIGS. 2A-2B presents an alignment among USP proteins derived from the indicated insect species. B. mori, Bombyx mori USP (GenBank Accession No. MC13750; SEQ ID NO:43); M. sexta, Manduca sexta USP (GenBank Accession No. MB64234; SEQ ID NO:44); C. fumiferana, Choristoneura fumiferana USP (GenBank Accession No. MC31795; SEQ ID NO:45); H. virescens, Heliothis virescens USP (SEQ ID NO:20); L. migratoria, Locusta migratoria USP (GenBank Accession No. AAF00981; SEQ ID NO:46); C. tentans, Chironomus tentans USP (GenBank Accession No. MC03056; SEQ ID NO:47); D. melanogaster, Drosophila melanogaster USP (GenBank Accession No. X53417; SEQ ID NO:48). The core DBD is underlined.

FIGS. 3A-3B presents an alignment among FTZ-F1 proteins derived from the indicated insect species. hv.bftz, Heliothis FTZ-F1 (SEQ ID NO:18); bmftz, Bombyx mori FTZ-F1 (GenBank Accession No. P49867; SEQ ID NO:49); dmftz, Drosophila βFTZ-F1 (GenBank Accession No. M98397; SEQ ID NO:32).

FIGS. 4A-4F presents an alignment among E75 proteins derived from the indicated insect species. D. melanogaster (A), Drosophila E75A (GenBank Accession No. A34598; SEQ ID NO:34); M. sexta (A), Manduca sexta E75A (GenBank Accession No. Q08893; SEQ ID NO:50); D. melanogaster (B), Drosophila E75B (GenBank Accession No. B34598; SEQ ID NO:51); M. sexta (B), Manduca sexta E75B (GenBank Accession No. C56591; SEQ ID NO:52); D. melanogaster (C), Drosophila E75C (GenBank Accession No. P13055; SEQ ID NO:53); C. fumiferana, Choristoneura fumiferana E75 (GenBank Accession No. 001639; SEQ ID NO:54); G. mellonella, Galleria mellonella E75 (GenBank Accession No. P50239; SEQ ID NO:55); M. ensis, Metapenaeus ensis E75 (GenBank Accession No. AAC71770; SEQ ID NO:56); H. virescens, Heliothis virescens (SEQ ID NO:18). The core DBD is underlined.

FIG. 5 is a bar graph that depicts percentage survival of Drosophila following injection of dsRNA corresponding to the indicated nuclear receptors as described in Example 5 herein below. Solid bar, DHR3; gray bar, DHR4; open bar, EGON; cross-hatched bar, FTZ-F1; wavy bar, DSF; checkerboard bar, DERR; stippled bar, FAX1; vertical line bar, FAX2; horizontal line bar, buffer (control).

FIG. 6 is a bar graph that depicts Drosophila larval lethality induced by overexpression of the indicated nuclear receptors. Gray bars, larvae that were not heat treated; solid bars, larvae that were heat treated at 0-2 hours post-hatching; open bars, larvae that were heat treated at 20-22 hours post-hatching; control, w¹¹¹⁸ larvae; DHR38, yw; P[hs-DHR38 w⁺]-II larvae; DHR39, w¹¹¹⁸; P[hs-DHR39-6 w⁺] or w¹¹¹⁸; P[hs-DHR39-3 w⁺] larvae; E75A, w¹¹¹⁸; +/SM5; P[hs-E75A w⁺]/TM3 or w¹¹¹⁸; +/SM5; P[hs-E75A w⁺]/TM3 larvae. Error bars indicate standard deviation.

### BRIEF DESCRIPTION OF SEQUENCES IN THE SEQUENCE LISTING

Odd-numbered SEQ ID NOs:1-31 are nucleotide sequences described in Table 1.

Even-numbered SEQ ID NOs:2-32 are protein sequences encoded by the immediately preceding nucleotide sequence, e.g., SEQ ID NO:2 is the protein encoded by the nucleotide sequence of SEQ ID NO:1, SEQ ID NO:4 is the protein encoded by the nucleotide sequence of SEQ ID NO:3, etc.

SEQ ID NOs:35-42 are PCR primers.

SEQ ID NOs:43-56 are protein sequences available from GenBank that are presented in the Figures for comparison with novel sequences disclosed herein.

TABLE 1
Sequence Listing Summary

| SEQ ID NO | description |
|---|---|
| 1-2 | Drosophila gm estroten-related receptor (gmDERR) |
| 3-4 | Drosophila estrogen-related receptor (DERR) |
| 5-6 | Drosophila fax-related receptor 1 (gmDFAX1) |
| 7-8 | Drosophila fax-related receptor 1 (DFAX1) |
| 9-10 | Drosophila gm fax-related receptor 2 (gmDFAX2) |
| 11-12 | Drosophila fax-related receptor 2 (DFAX2) |
| 13-14 | Drosophila hormone receptor 4 (DHR4) |
| 15-16 | Drosophila dissatisfaction (DSF) |
| 17-18 | Heliothis fushi tarazu factor 1 (FTZ-F1) |
| 19-20 | Heliothis ecdysone-inducible protein 75 (E75) |
| 21-22 | Heliothis ultraspiracle (USP) |
| 23-24 | Heliothis hepatic nuclear factor 4 (HNF4) |
| 25-26 | Heliothis hormone receptor 3 (HR3) |
| 27-28 | Drosophila hormone receptor 38 (DHR38) |
| 29-30 | Drosophila hormone receptor 39 (DHR39) |
| 31-32 | Drosophila fushi tarazu factor 1, β isoform (βFTZ-F1) |
| 33-34 | Drosophila E75A |
| 35 | βFTZ-F1 degenerate forward primer A |
| 36 | βFTZ-F1 degenerate forward primer B |
| 37 | βFTZ-F1 degenerate reverse primer A |
| 38 | βFTZ-F1 degenerate reverse primer B |
| 39 | βFTZ-F1 forward primer A |
| 40 | βFTZ-F1 forward primer B |
| 41 | βFTZ-F1 reverse primer A |
| 42 | βFTZ-F1 reverse primer B |
| 43 | Bombyx ultraspiracle (USP) |
| 44 | Manduca ultraspiracle (USP) |
| 45 | Chorisoneura ultraspiracle (USP) |
| 46 | Locusta ultraspiracle (USP) |
| 47 | Chironomus ultraspiracle (USP) |
| 48 | Drosophila ultraspiracle (USP) |
| 49 | Bombyx FTZ-F1 |
| 50 | Manduca E75A |
| 51 | Drosophila E75B |
| 52 | Manduca E75B |
| 53 | Drosophila E75C |
| 54 | Choristoneura E75 |
| 55 | Galleria E75 |
| 56 | Matapenaeus E75 |

### DETAILED DESCRIPTION OF THE INVENTION

I. Definitions

While the following terms are believed to be well understood by one of ordinary skill in the art, the following definitions are set forth to facilitate explanation of the invention.

I.A. Nucleic Acids

The nucleic acid molecules provided by the present invention include the isolated nucleic acid molecules of any one of SEQ ID NOs:1, 5, 9, 13, 17, 19, 21, 23, and 25; sequences substantially identical to sequences of any one of SEQ ID NOs:1, 5, 9, 13, 17, 19, 21, 23, and 25; conservative variants thereof, subsequences and elongated sequences thereof, complementary DNA molecules, and corresponding RNA molecules. The present invention also encompasses genes, cDNAs, chimeric genes, and vectors comprising disclosed nuclear receptor nucleic acid sequences.

The term “nucleic acid molecule” refers to deoxyribonucleotides or ribonucleotides and polymers thereof in either single- or double-stranded form. Unless specifically limited, the term encompasses nucleic acids containing known analogues of natural nucleotides that have similar properties as the reference natural nucleic acid. Unless otherwise indicated, a particular nucleotide sequence also implicitly encompasses conservatively modified variants thereof (e.g., degenerate codon substitutions), complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism.

The term “isolated”, as used in the context of a nucleic acid molecule, indicates that the nucleic acid molecule exists apart from its native environment and is not a product of nature. An isolated DNA molecule can exist in a purified form or can exist in a non-native environment such as a transgenic host cell.

The term “purified”, when applied to a nucleic acid, denotes that the nucleic acid is essentially free of other cellular components with which it is associated in the natural state. Preferably, a purified nucleic acid molecule is a homogeneous dry or aqueous solution. The term “purified” denotes that a nucleic acid gives rise to essentially one band in an electrophoretic gel. Particularly, it means that the nucleic acid is at least about 50% pure, more preferably at least about 85% pure, and most preferably at least about 99% pure.

The term “substantially identical”, in the context of two nucleotide sequences, refers to two or more sequences or subsequences that have at least 60%, preferably about 70%, more preferably about 80%, more preferably about 90-95%, and most preferably about 99% nucleotide identity, when compared and aligned for maximum correspondence, as measured using one of the following sequence comparison algorithms (described herein below under the heading “Nucleotide and Amino Acid Sequence Comparisons” or by visual inspection. Preferably, the substantial identity exists in nucleotide sequences of at least 50 residues, more preferably in nucleotide sequence of at least about 100 residues, more preferably in nucleotide sequences of at least about 150 residues, and most preferably in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

A preferred nucleotide sequence employed for hybridization studies or assays includes probe sequences that are complementary to or mimic at least an about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any of those set forth as SEQ ID NOs:1, 5, 9, 13, 17, 19, 21, 23, and 25. Such fragments can be readily prepared by, for example, directly synthesizing the fragment by chemical synthesis, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC, SM NaCl at 65° C. An example of stringent wash conditions is 15 minutes in 0.2×SSC buffer at 65° C. (See Sambrook et al., eds (1989) Molecular Cloning: A Laboratory Manual, Cold Spring Harbor Laboratory Press, Cold Spring Harbor, N.Y. for a description of SSC buffer). Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the present invention: a probe nucleotide sequence preferably hybridizes to a target nucleotide sequence in 7% sodium dodecyl sulfate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulfate (SDS), 0.5M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, are biologically functional equivalents, or are immunologically cross-reactive. These terms are defined further under the heading “Polypeptides” herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues (Batzer et al. (1991) Nuc Acids Res 19:5081; Ohtsuka et al. (1985) J Biol Chem 260: 2605-2608; Rossolini et al. (1994) Mol Cell Probes 8: 91-98).

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression” generally refers to the cellular processes by which a biologically active polypeptide is produced from a DNA sequence.

The present invention also encompasses chimeric genes comprising the disclosed nuclear receptor sequences. The term “chimeric gene”, as used herein, refers to a promoter region operatively linked to a nuclear receptor coding sequence, a nucleotide sequence producing an antisense RNA molecule, a RNA molecule having tertiary structure, such as a hairpin structure, or a double-stranded RNA molecule.

The term “operatively linked”, as used herein, refers to a promoter region that is connected to a nucleotide sequence in such a way that the transcription of that nucleotide sequence is controlled and regulated by that promoter region. Techniques for operatively linking a promoter region to a nucleotide sequence are known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native cis-regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

The term “transcription factor” generally refers to a protein that modulates gene expression by interaction with the cis-regulatory element and cellular components for transcription, including RNA Polymerase, Transcription Associated Factors (TAFs), chromatin-remodeling proteins, and any other relevant protein that impacts gene transcription.

The present invention further includes vectors comprising the disclosed nuclear sequences, including plasmids, cosmids, and viral vectors. The term “vector”, as used herein refers to a DNA molecule having sequences that enable its replication in a compatible host cell. A vector also includes nucleotide sequences to permit ligation of nucleotide sequences within the vector, wherein such nucleotide sequences are also replicated in a compatible host cell. A vector can also mediate recombinant production of a nuclear receptor polypeptide, as described further herein below. A preferred host cell is a bacterial cell, an insect cell, or a plant cell.

Nucleic acids of the present invention can be cloned, synthesized, recombinantly altered, mutagenized, or combinations thereof. Standard recombinant DNA and molecular cloning techniques used to isolate nucleic acids are known in the art. Exemplary, non-limiting methods are described by Sambrook et al., eds (1989) Molecular Cloning, Cold Spring Harbor Laboratory Press, Cold Spring Harbor, N.Y.; by Silhavy et al. (1984) Experiments with Gene Fusions, Cold Spring Harbor Laboratory Press, Cold Spring Harbor, N.Y.; by Ausubel et al. (1992) Current Protocols in Molecular Biology, John Wylie and Sons, Inc., New York, N.Y.; and by Glover, ed (1985) DNA Cloning: A Practical Approach, MRL Press, Ltd., Oxford, United Kingdom. Site-specific mutagenesis to create base pair changes, deletions, or small insertions are also known in the art as exemplified by publications. See e.g., Adelman et al. (1983) DNA 2:183; Sambrook et al., eds (1989) Molecular Cloning, Cold Spring Harbor Laboratory Press, Cold Spring Harbor, N.Y.

Sequences detected by methods of the invention can be detected, subcloned, sequenced, and further evaluated by any measure known in the art using any method usually applied to the detection of a specific DNA sequence including but not limited to dideoxy sequencing, PCR, oligomer restriction (Saiki et al. (1985) Bio/Technology 3:1008-1012), allele-specific oligonucleotide (ASO) probe analysis (Conner et al. (1983) Proc Natl Acad Sci USA 80:278), and oligonucleotide ligation assays (OLAs) (Landgren et al. (1988) Science 241:1007). See also Landgren et al. (1988) Science 242:229-237.

I.B. Polypeptides

The polypeptides provided by the present invention include the isolated polypeptides set forth as SEQ ID NOs:2, 6, 10, 14, 18, 20, 22, 24, and 26; polypeptides substantially identical to SEQ ID NOs:2, 6, 10, 14, 18, 20, 22, 24, and 26; nuclear receptor polypeptide fragments (preferably biologically functional fragments, e.g. the domains described herein), fusion proteins comprising the disclosed nuclear receptor amino acid sequences, biologically functional analogs, and polypeptides that cross-react with an antibody that specifically recognizes a disclosed nuclear receptor polypeptide.

The term “isolated”, as used in the context of a polypeptide, indicates that the polypeptide exists apart from its native environment and is not a product of nature. An isolated polypeptide can exist in a purified form or can exist in a non-native environment such as, for example, in a transgenic host cell.

The term “purified”, when applied to a polypeptide, denotes that the polypeptide is essentially free of other cellular components with which it is associated in the natural state. Preferably, a polypeptide is a homogeneous solid or aqueous solution. Purity and homogeneity are typically determined using analytical chemistry techniques such as polyacrylamide gel electrophoresis or high performance liquid chromatography. A polypeptide that is the predominant species present in a preparation is substantially purified. The term “purified” denotes that a polypeptide gives rise to essentially one band in an electrophoretic gel. Particularly, it means that the polypeptide is at least about 50% pure, more preferably at least about 85% pure, and most preferably at least about 99% pure.

The term “substantially identical” in the context of two or more polypeptide sequences is measured as polypeptide sequences having about 35%, or 45%, or preferably from 45-55%, or more preferably 55-65% of identical or functionally equivalent amino acids. Even more preferably, two or more “substantially identical” polypeptide sequences will have about 70%, or even more preferably about 80%, still more preferably about 90%, still more preferably about 95%, and most preferably about 99% identical or functionally equivalent amino acids. Percent “identity” and methods for determining identity are defined herein below under the heading “Nucleotide and Amino Acid Sequence Comparisons”.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites. See Henikoff et al. (2000) Electrophoresis 21(9):1700-1706; Huang et al. (2000) Pac Symp Biocomput 230-241; Saqi et al. (1999) Bioinformatics 15(6):521-522; and Barton (1998) Acta Crystallogr D Biol Crystallogr 54:1139-1146.

The term “functionally equivalent” in the context of amino acid sequences is known in the art and is based on the relative similarity of the amino acid side-chain substituents. See Henikoff & Henikoff (2000) Adv Protein Chem 54:73-97. Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte et al. (1982) J Mol Biol 157:105). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those that are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101 states that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those that are within ±1 of the original value are particularly preferred, and those within ±0.5 of the original value are even more particularly preferred.

The present invention also encompasses nuclear receptor polypeptide fragments or functional portions of a nuclear receptor polypeptide. Such functional portion need not comprise all or substantially all of the amino acid sequence of a native nuclear receptor gene product. The term “functional” includes any biological activity or feature of nuclear receptor, including immunogenicity.

The present invention also includes longer sequences of a nuclear receptor polypeptide, or portion thereof. For example, one or more amino acids can be added to the N-terminus or C-terminus of a nuclear receptor polypeptide. Fusion proteins comprising nuclear receptor polypeptide sequences are also provided within the scope of the present invention. Methods of preparing such proteins are known in the art.

The present invention also encompasses functional analogs of a nuclear receptor polypeptide. Functional analogs share at least one biological function with a nuclear receptor polypeptide. An exemplary function is immunogenicity. In the context of amino acid sequence, biologically functional analogs, as used herein, are peptides in which certain, but not most or all, of the amino acids can be substituted. Functional analogs can be created at the level of the corresponding nucleic acid molecule, altering such sequence to encode desired amino acid changes. In one embodiment, changes can be introduced to improve a biological function of the polypeptide, e.g., to improve the antigenicity of the polypeptide. In another embodiment, a nuclear receptor polypeptide sequence is varied so as to assess the activity of a mutant nuclear receptor polypeptide.

The present invention also encompasses recombinant production of the disclosed nuclear receptor polypeptides. Briefly, a nucleic acid sequence encoding a nuclear receptor polypeptide, or portion thereof, is cloned into an expression cassette, the cassette is introduced into a host organism, where it is recombinantly produced.

The term “expression cassette” as used herein means a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The expression cassette comprising the nucleotide sequence of interest can be chimeric. The expression cassette can also be one that is naturally occurring but has been obtained in a recombinant form useful for heterologous expression.

The expression of the nucleotide sequence in the expression cassette can be under the control of a constitutive promoter or an inducible promoter that initiates transcription only when the host cell is exposed to some particular external stimulus. Exemplary promoters include Simian virus 40 early promoter, a long terminal repeat promoter from retrovirus, an action promoter, a heat shock promoter, and a metallothien protein. In the case of a multicellular organism, the promoter and promoter region can direct expression to a particular tissue or organ or stage of development. Suitable expression vectors which can be used include, but are not limited to, the following vectors or their derivatives: viruses such as vaccinia virus or adenovirus, baculovirus vectors, yeast vectors, bacteriophage vectors (e.g., lambda phage), plasmid and cosmid DNA vectors, and transposon-mediated transformation vectors.

The term “host cell”, as used herein, refers to a cell into which a heterologous nucleic acid molecule has been introduced. Transformed cells, tissues, or organisms are understood to encompass not only the end product of a transformation process, but also transgenic progeny thereof.

A host cell strain can be chosen which modulates the expression of the inserted sequences, or modifies and processes the gene product in the specific fashion desired. For example, different host cells have characteristic and specific mechanisms for the translational and post-transactional processing and modification (e.g., glycosylation, phosphorylation of proteins). Appropriate cell lines or host systems can be chosen to ensure the desired modification and processing of the foreign protein expressed. Expression in a bacterial system can be used to produce a non-glycosylated core protein product. Expression in yeast will produce a glycosylated product. Expression in insect cells can be used to ensure “native” glycosylation of a heterologous protein.

Expression constructs are transfected into a host cell by any standard method, including electroporation, calcium phosphate precipitation, DEAE-Dextran transfection, liposome-mediated transfection, transposon-mediated transformation and infection using a retrovirus. The nuclear receptor-encoding nucleotide sequence carried in the expression construct can be stably integrated into the genome of the host or it can be present as an extrachromosomal molecule.

Isolated polypeptides and recombinantly produced polypeptides can be purified and characterized using a variety of standard techniques that are known to the skilled artisan. See e.g., Ausubel et al. (1992) Current Protocols in Molecular Biology, John Wylie & Sons, Inc., New York, N.Y.; Bodanszky, et al. (1976) Peptide Synthesis, John Wiley and Sons, Second Edition, New York, N.Y.; and Zimmer et al. (1993) Peptides, pp. 393-394, ESCOM Science Publishers, B. V.

I.C. Nucleotide and Amino Acid Sequence Comparisons

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain at least some of biological activity of the natural gene, gene product, or sequence. Such sequences include “mutant” sequences, or sequences wherein the biological activity is altered to some degree but retains at least some of the original biological activity. The term “naturally occurring”, as used herein, is used to describe a composition that can be found in nature as distinct from being artificially produced by man. For example, a protein or nucleotide sequence present in an organism, which can be isolated from a source in nature and which has not been intentionally modified by man in the laboratory, is naturally occurring.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981) Adv Appl Math 2:482, by the homology alignment algorithm of Needleman & Wunsch (1970) J Mol Biol 48:443, by the search for similarity method of Pearson & Lipman (1988) Proc Natl Acad Sci USA 85:2444-2448, by computerized implementations of these algorithms (GAP, BESTFIT, FASTA, and TFASTA in the Wisconsin Genetics Software Package, Genetics Computer Group, Madison, Wis.), or by visual inspection. See generally, Ausubel et al. (1992) Current Protocols in Molecular Biology, John Wylie & Sons, Inc., New York, N.Y.

A preferred algorithm for determining percent sequence identity and sequence similarity is the BLAST algorithm, which is described in Altschul et al. (1990) J Mol Biol 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff (1989) Proc Natl Acad Sci USA 89:10915.

In addition to calculating percent sequence identity, the BLAST algorithm also performs a statistical analysis of the similarity between two sequences. See e.g., Karlin & Altschul (1993) Proc Natl Acad Sci USA 90:5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

I.D. Antibodies

Also provided is an antibody that specifically binds an insect nuclear receptor polypeptide of the present invention. The term “antibody” indicates an immunoglobulin protein, or functional portion thereof, including a polyclonal antibody, a monoclonal antibody, a chimeric antibody, a single chain antibody, Fab fragments, and a Fab expression library. “Functional portion” refers to the part of the protein that binds a molecule of interest. In a preferred embodiment, an antibody of the invention is a monoclonal antibody. Techniques for preparing and characterizing antibodies are known in the art. See e.g., Harlow & Lane (1988) Antibodies: A Laboratory Manual, Cold Spring Harbor Laboratory Press, Cold Spring Harbor, N.Y. A monoclonal antibody of the present invention can be readily prepared through use of well-known techniques such as the hybridoma techniques exemplified in U.S. Pat. No. 4,196,265 and the phage-displayed techniques disclosed in U.S. Pat. No. 5,260,203.

The phrase “specifically (or selectively) binds to an antibody”, or “specifically (or selectively) immunoreactive with”, when referring to a protein or peptide, refers to a binding reaction which is determinative of the presence of the protein in a heterogeneous population of proteins and other biological materials. Thus, under designated immunoassay conditions, the specified antibodies bind to a particular protein and do not show significant binding to other proteins present in the sample. Specific binding to an antibody under such conditions can require an antibody that is selected based on its specificity for a particular protein. For example, antibodies raised to a protein with an amino acid sequence encoded by any of the nucleic acid sequences of the invention can be selected to obtain antibodies specifically immunoreactive with that protein and not with unrelated proteins.

The use of a molecular cloning approach to generate antibodies, particularly monoclonal antibodies, and more particularly single chain monoclonal antibodies, are also provided. The production of single chain antibodies has been described in the art. See e.g., U.S. Pat. No. 5,260,203. For this approach, combinatorial immunoglobulin phagemid libraries are prepared from RNA isolated from the spleen of the immunized animal, and phagemids expressing appropriate antibodies are selected by panning on tissue that expresses the polypeptide. The advantages of this approach over conventional hybridoma techniques are that approximately 10⁴ times as many antibodies can be produced and screened in a single round, and that new specificities are generated by heavy (H) and light (L) chain combinations in a single chain, which further increases the chance of finding appropriate antibodies. Thus, an antibody of the present invention, or a “derivative” of an antibody of the present invention, pertains to a single polypeptide chain binding molecule which has binding specificity and affinity substantially identical to the binding specificity and affinity of the light and heavy chain aggregate variable region of an antibody described herein.

The term “immunochemical reaction”, as used herein, refers to any of a variety of immunoassay formats used to detect antibodies specifically bound to a particular protein, including but not limited to competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (e.g., using colloidal gold, enzyme or radioisotope labels), Western blot analysis, precipitation reactions, agglutination assays (e.g., gel agglutination assays, hemagglutination assays), complement fixation assays, immunofluorescence assays, protein A assays, and immunoelectrophoresis assays, etc. See Harlow & Lane (1988) Antibodies: A Laboratory Manual, Cold Spring Harbor Laboratory Press, Cold Spring Harbor, N.Y. for a description of immunoassay formats and conditions.

I.E. Transgenic Organisms

It is also within the scope of the present invention to prepare a transgenic organism to express a transgene comprising nucleic acid sequences of the present invention. The term “transgenic organism”, indicates an organism comprising a germline insertion of a heterologous nucleic acid. A transgenic organism can be an animal or a plant. Transgenic organisms of the present invention are understood to encompass not only the end product of a transformation method, but also transgenic progeny thereof.

The term “transgene”, as used herein indicates a heterologous nucleic acid molecule that has been transformed into a host cell. For intended use in the creation of a transgenic organism, the transgene can include genomic sequences of the host organism at a selected locus or site of transgene integration to mediate a homologous recombination event. A transgene further comprises nucleic acid sequences of interest, for example a targeted modification of the gene residing within the locus, a reporter gene, or a expression cassette, each defined herein above.

II. Nuclear Receptors

II.A. Conserved Features

The steroid/nuclear receptor superfamily comprises soluble receptor proteins that function as ligand-inducible transcription factors. Nuclear receptor polypeptides are characterized by the presence of five or six evolutionarily conserved domains: A/B, C, D, E and F (Evans (1988) Science 240:889-895; Laudet et al. (1992) EMBO J. 11:1003-1013). Various functions are ascribed to each domain, e.g., “A/B” refers to the transactivation domain, “C” refers to the DNA binding domain, “D” refers to the hinge/linker domain, “E” refers to the ligand binding domain, and “F” refers to the variable C-terminal domain that is present in some receptor polypeptides. In general, the domains can be modular in that the function of an individual domain is preserved in the context of a chimeric protein. See e.g., Green & Chambon (1987) Nature 325:75-78; Green et al. (1988) EMBO J. 7:3037-3044; Giguere et al. (1987) Nature 330:624-629.

The “A/B” region is of variable size and poorly conserved. In some cases, the A/B region has a transcriptional activation function. See Ptashne (1988) Nature 335:683-689; Hadzic et al. (1995) Mol Cell Biol 15:4507-4517; Pakdel et al. (1993) Mol Endocrinol 7:1408-1417; Thompson et al. (1989) Proc Natl Acad Sci USA 86:3493-3498. Amino acids that confer a transcriptional activation function can facilitate repeated transcription initiation events leading to greater levels of gene expression from a target gene.

The “C” (DNA binding) domain is a highly conserved region of approximately 198 nucleotides that encode an approximately 66 amino acid and polypeptide that comprises two Cys₂-Cys₂ zinc finger DNA binding motifs (Danielsen et al. (1989) Cell 57:1131-1138; Green et al. (1988) EMBO J. 7:3037-3044; Umesono & Evans (1989) Cell 57:1139-1146). The DNA-binding domain also facilitates receptor dimerization (Perlmann et al. (1993) Genes Dev 7:1411-1422; Zechel et al. (1994) EMBO J. 13:1414-1424; Mader et al. (1993) EMBO J. 12:5029-5041; Hard et al. (1990) Science 249:397-404). CH4 The term “core DNA binding domain” is defined as the 66 amino acid sequence that generally begins with a conserved CYS residue and ends with conserved residues GLU-MET. See e.g., Rastinejad (1998) in Freedman, ed, Molecular Biology of Steroid and Nuclear Hormone Receptors, pp. 107, Birkhäuser, Boston, Mass. The DNA binding domain is also characterized by a conserved three-dimensional fold when backbone atoms are compared (Rastinejad et al. (1995) Nature 375:203-211).

The affinity of the DNA binding domain for a DNA recognition site can be influenced by residues that are N-terminal or C-terminal to the core DNA-binding domain. See e.g., Ueda et al. (1992) Mol Cell Biol 12:5667-5672; Rastinejad (1998) in Freedman, ed, Molecular Biology of Steroid and Nuclear Hormone Receptors, pp. 103-131, Birkhäuser, Boston, Mass. and references cited therein. In particular, the term “P Box” refers to sequences that are adjacent to the C-terminal end of the DNA binding domain that facilitate binding to the 5′ end, generally an A/T-rich sequence of a target response element.

The “D” (hinge/linker) domain is located between the DNA binding domain and the ligand binding domain and can contribute to the strength and specificity of DNA-binding. See e.g., Ueda et al. (1992) Mol Cell Biol 12:5667-5672; Rastinejad (1998) in Freedman, ed, Molecular Biology of Steroid and Nuclear Hormone Receptors, pp. 103-131, Birkhäuser, Boston, Mass. and references cited therein. In some cases, an extended DNA binding site that include sequences within the hinge domain can enable monomeric binding (e.g., Ueda et al., (1992) Mol Cell Biol 112:5667-5672; Wilson et al. (1992) Science 256:106-110). The hinge region is also implicated in conferring flexibility between the ligand and DNA binding domains (Bourguet et al. (1995) Nature 375:377-382; Wagner et al. (1995) Nature 378:690-697).

The “E” (ligand binding) domain, as used herein, is interchangeably referred to as the “ligand binding domain” or the “hormone binding domain”. The ligand binding domain comprises a hydrophobic pocket that enables regulation of nuclear receptor activity by small chemical ligands. Unlike the DNA binding domain, the ligand binding domain is not clearly delineated by amino acid sequence. However, the general position of the ligand binding domain is conserved at the carboxyl end of the protein. The ligand binding domain is further characterized by a conserved tertiary structure (Wurtz et al. (1996) Nat Struct Biol 3:87-94).

The functional ligand binding domain within the carboxyl region of a nuclear receptor polypeptide is operationally defined as the amino acids required for high affinity binding of any ligand and can be determined according to methods known in the art. See e.g., Rusconi & Yamamoto (1987) EMBO J 6:1309-1315; Zhang et al. (1996a) Mol Endocrinol 10:24-34; Lanz & Rusconi (1994) Endocrinology 135:2183-2194; Xu et al. (1996) J Biol Chem 271:21430-21438; Zhang et al. (1996b) J Biol Chem 271:14825-14833).

The ligand binding domain also mediates receptor dimerization. See Simons in Freedman, ed, Molecular Biology of Steroid and Nuclear Hormone Receptors, pp. 35-104, Birkhäuser, Boston, Mass., and references cited therein. A series of heptad repeats in the ligand binding domain, specifically helix 10 with some contribution from helix 9, forms the main dimer interface (Forman et al. (1989) Mol Endocrinol 3:1610-1626; Forman & Samuels (1990) Mol Endocrinol 4:1293-1301; Bourguet et al. (1995b) Nature 375:377-382; Wurtz et al. (1996) Nat Struct Biol 3:87-94).

II.B. Identification of Novel Drosophila Nuclear Receptors

The present invention provides novel Drosophila nuclear receptor nucleic acid and polypeptide sequences. Preferably, a Drosophila nuclear receptor nucleic acid molecule of the present invention comprises the sequence set forth as any one of the SEQ ID NOs:1, 5, 9, 13, 17, 19, 21, 23, and 25; or a nucleic acid molecule that is substantially identical to any one of SEQ ID NOs:1, 5, 9, 13, 17, 19, 21, 23, and 25. Also preferably, a nuclear receptor polypeptide of the present invention comprises an amino acid sequence set forth as any one of SEQ ID NOs:2, 6, 10, 14, 18, 20, 22, 24, and 26; or a polypeptide that is substantially identical to any one of SEQ ID NOs:2, 6, 10, 14, 18, 20, 22, 24, and 26.

To identify new Drosophila proteins, a database of predicted proteins (referred to herein as “the GeneMark database”) was assembled using the GeneMark program (Borodovsky & McIninch (1993) Computers & Chemistry 17:123-133) and template 50 kb genomic sequence scaffolds generated by Celera Corporation (Rockville Md.). A profile Hidden Markov Model (HMM) was built using Drosophila and C. elegans sequences as described in Example 1. The profile HMM was used to query predicted protein databases, including the GeneMark database and a predicted protein database generated by Celera using an alternative protein prediction program (referred to herein as “the Celera database”).

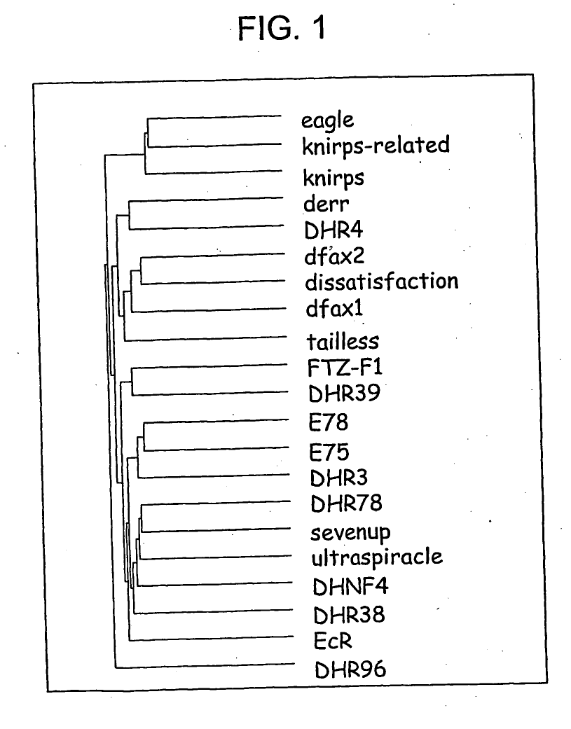

Three new nuclear receptor sequences were identified (SEQ ID NOs:2, 6, and 10) in the GeneMark database, and three similar sequences were identified in the Celera database (SEQ ID NOs:4, 8, and 12) (FIG. 1). A fourth nuclear receptor, which was designated DHR4 based on its close homology to Tenbrio and Manduca THR4 sequences, was variably predicted based on the sequences of both databases as well as other genomic clones (Accession No. AL035245). In contrast to the predicted cDNA and protein sequences, the DHR4 nucleotide sequence disclosed herein (SEQ ID NO:13) comprises an isolated DHR4 cDNA, which encodes a DHR4 protein (SEQ ID NO:14) that is different than the predicted protein sequences noted herein above.

Nuclear receptor cDNAs that encode the novel nuclear receptors identified in the GeneMark database were predicted by using the predicted receptor polypeptides to perform a TBLASTN search the genomic sequence. Nucleotide sequences retrieved by this search were assembled as the predicted nuclear receptor cDNAs. The corresponding genes were subsequently cloned as described in Example 2.

The novel Drosophila nuclear receptor sequences were named according to the most closely related nuclear receptor as shown in Table 2.

TABLE 2
BLAST Analysis of New Nuclear Receptors

| | reference | (ACCESSION) | (bits) | | | |
|---|---|---|---|---|---|---|
| 2 | DERR | human ERRB2, complete cds (AF094517) | 266 | 6e−70 | 158/375 (42%) | 202/375 (53%) |
| 6 | DFAX1 | C. elegans FAX-1, complete cds (AF176087) | 118 | 3e−25 | 57/104 (54%) | 66/104 (62%) |
| 10 | DFAX2 | C. elegans FAX-1, complete cds (AF176087) | 150 | 4e−35 | 72/88 (81%) | 72/88 (81%) |
| 14 | DHR4 | D. melanogaster BAC clone, predicted cds (AL035245) | 605 | e−171 | 358/731 (48%) | 358/731 (48%) |
| | | D. melanogaster genomic scaffold, predicted cds (AE003422) | 506 | e−142 | 271/331 (81%) | 271/331 (81%) |
| | | Bombyx GRF, complete cds (AF124981) | 367 | e−100 | 194/308 (62%) | 234/308 (74%) |

II. C. Identification of New Heliothis Nuclear Receptors

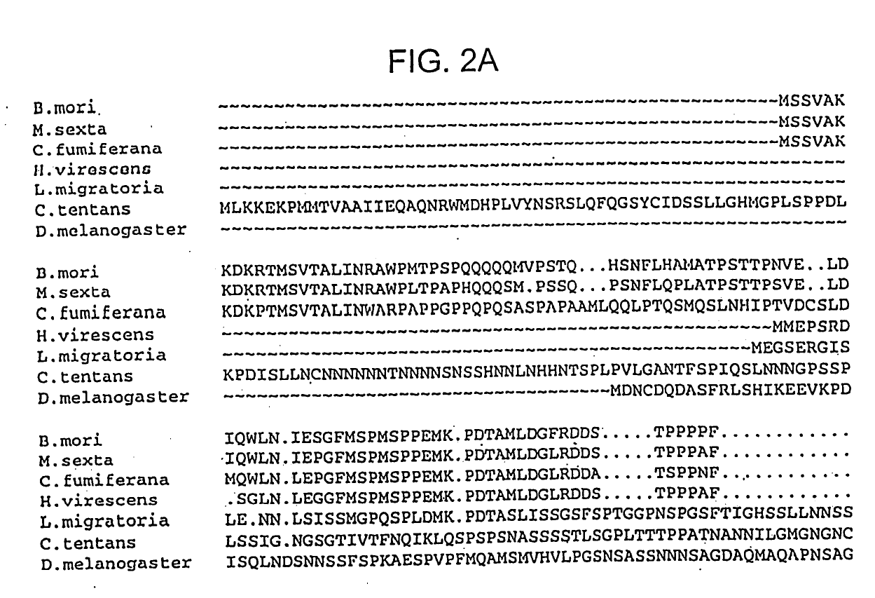

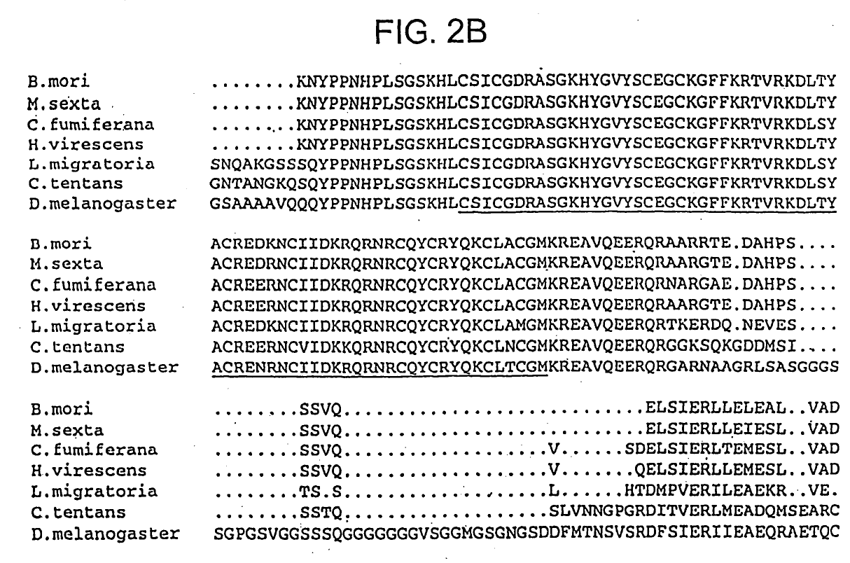

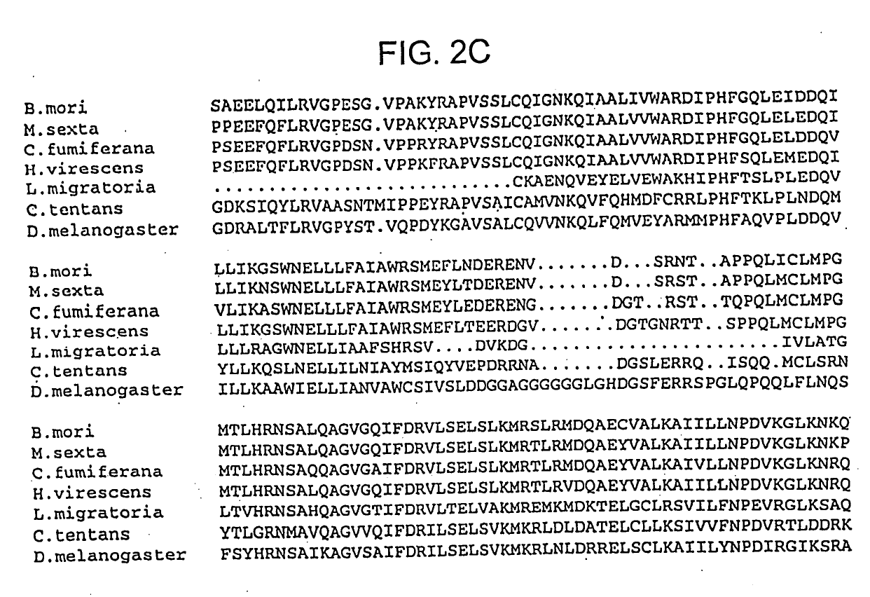

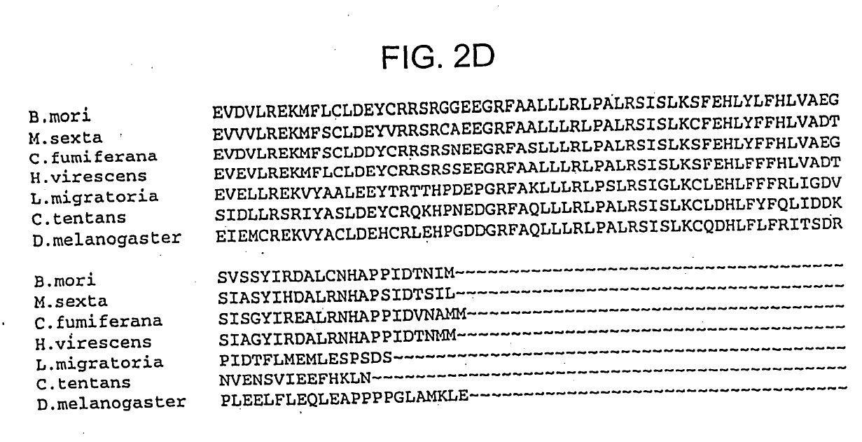

To identify new nuclear receptors in a pest insect, Heliothis virescens (hereinafter “Heliothis”), a cDNA library derived from Heliothis transcripts was screened using a mixture of labeled Drosophila nuclear receptor sequences as probe, as described in Example 3. Additional Heliothis nuclear receptor sequences were obtained by PCR using degenerate primers designed according to Drosophila nuclear receptor sequences, as described in Example 4. Heliothis nuclear receptor fragments derived from both methods were assembled by recognition of overlapping sequence. New Heliothis nuclear receptors were named based on the most closely related insect nuclear receptor (Table 3 and FIGS. 2-4). Heliothis FTZ-F1 is most similar to the β isoform encoded by Drosophila FTZ-F1, βFTZ-F1, in that it lacks an extended N-terminal domain characteristic of the αFTZ-F1 isoform.

TABLE 3
BLAST Analysis of Heliothis Nuclear Receptors

| | | Drosophila | | | | |
|---|---|---|---|---|---|---|
| | inventor's | homologue | Score | | | |
| SEQ ID NO. | reference | (ACCESSION) | (bits) | E value | Identities | Positives |
| 18 | FTZ-F1 | AAF49231 | 380 | e−104 | 182/236 (77%) | 202/236 (85%) |
| 20 | E75 | P17671 | 631 | e−180 | 312/391 (79%) | 343/391 (86%) |
| 22 | USP | X52591 | 337 | 2e−91 | 186/413 (45%) | 241/413 (58%) |
| 24 | HNF4 | P49866 | 311 | 4e−84 | 154/216 (71%) | 177/216 (81%) |
| 26 | HR3 | M90806 | 261 | 5e−69 | 145/219 (66%) | 160/219 (72%) |

III. Functional Analysis of Drosophila melanogaster Nuclear Receptors

Many insect pests inflict plant damage by the feeding activity during larval stages. Therefore, functional analyses to assess phenotypes associated with modulation of a nuclear receptor during larval development can be used to identify candidate insecticide targets. The present invention discloses nuclear receptors whose regulation is relevant to larval viability. Thus, modulators that alter nuclear receptor activity in a manner analogous to the changes resulting from genetic manipulations described herein below, can be useful as insecticide compositions.

III.A. Loss-Of-Function Analyses

RNA-mediated interference (RNAi) is a recently discovered method to determine gene function in a number of organisms, wherein double-stranded RNA (dsRNA) directs gene-specific, post-transcriptional silencing. See e.g., Kuwabara & Olson (2000) Parasitol Today 16(8): 347-349; Bass (2000) Cell 101 (3):235-238; Hunter (2000) Curr Biol 10(4):R137-140; Bosher & Labouesse (2000) Nat Cell Biol 2(2):E31-36; Sharp (1999) Genes Dev 13(2):139-141. The double-stranded RNA molecule can be synthesized in vitro and then introduced into the organism by injection or other methods. Alternatively, a heritable transgene exhibiting dyad symmetry can provide a transcript that folds as a hairpin structure. Methods for examining gene functions using dsRNAi in Drosophila are disclosed in Example 5 and further in Kennerdell & Carthew (2000) Nat Biotech 18(8):896-898; Lam & Thummel (2000) Curr Biol 10(16):957-963; Misquitta & Paterson (1999) Proc Natl Acad Sci USA 96 (4):1451-1456.

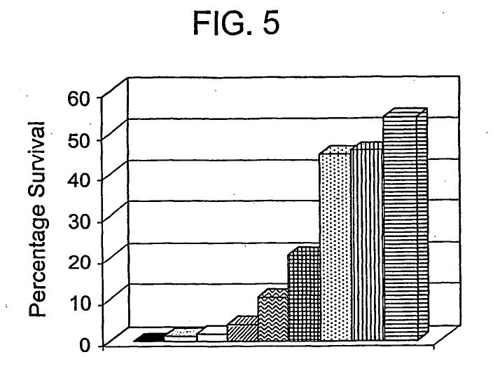

The present invention discloses RNA-mediated interference of Drosophila nuclear receptors DERR (SEQ ID NO:2), DFAX1 (SEQ ID NO:6), DFAX2 (SEQ ID NO:10), DHR4 (SEQ ID NO:14), DSF (GenBank Accession No. AF10667, SEQ ID NO:16), FTZ-F1 (GenBank Accession M98387), EGON (GenBank Accession No. D43634), and DHR3 (GenBank Accession No. M90806) (FIG. 5). Double-stranded RNA complementary to each nuclear receptor sequence was synthesized in vitro and injected into early Drosophila embryos, as described in Example 5. Development of injected embryos was assessed by scoring: (a) morphological criteria using a light microscope (Campos-Ortega & Hartenstein (1985) The Embryonic Development of Drosophila melanogaster, Springer-Verlag, Berlin), (b) embryo hatching to become a larvae, (c) puparium formation, and (d) eclosion of the pupae as an adult fly, as indicated in Table 4 herein below. Buffer-injected embryos were injected and monitored in parallel as a control. The percentage of embryos injected with dsRNA that survive to the adult stage is depicted in FIG. 5.

TABLE 4
Results of dsRNAi Analysis

| | | # embryos | | | | | | | |
|---|---|---|---|---|---|---|---|---|---|
| | | showing | | | | | | | |
| | # eggs | morphological | | | | % embryonic | % larval | % pupal | |
| dsRNA | injected | development | # larvae | # pupae | # adults | lethality | lethality | lethality | viable |
| DHR3 | 212 | 181 | 15 | 0 | 0 | 92% | 8% | 0 | 0 |
| DHR4 | 405 | 352 | 7 | 7 | 4 | 98% | 0 | <1% | 1% |
| EGON | 166 | 127 | 93 | 3 | 2 | 27% | 71% | <1% | 2% |
| FTZ-F1 | 373 | 235 | 13 | 10 | 9 | 94% | 1% | <1% | 4% |
| DSF | 103 | 88 | 13 | 9 | 99 | 85% | 5% | 0 | 10% |
| DERR | 304 | 270 | 223 | 59 | 55 | 17% | 61% | 1% | 20% |
| DFAX1 | 333 | 273 | 147 | 124 | 123 | 46% | 8% | <1% | 46% |
| DFAX2 | 468 | 418 | 251 | 199 | 192 | 40% | 12% | 2% | 46% |
| buffer | 941 | 806 | 580 | 500 | 433 | 28% | 10% | 8% | 54% |

Percentage lethality at each developmental stage was calculated as follows:
% embryonic lethality = [(# embryos showing morphological development) − (# larvae)]/# embryos showing morphological development
% larval lethality = [(# larvae) − (# pupae)]/# embryos showing morphological development
% pupal lethality = [(# pupae) − (# adults)]/# embryos showing morphological development
% viable = # adults/# embryos showing morphological development

Injection of double-stranded DHR3, FTZ-F1, and EGON RNA conferred significant embryonic lethality (92% and 94% respectively). Embryos injected with double-stranded EGON RNA also showed lethality during larval stages (71%). The observed lethal phases resulting from loss of DHR3, FTZ-F1, and EGON, as determined using dsRNAi, are consistent with published loss-of-function phenotypes for DHR3 (Carney et al. (1997) Proc Natl Acad Sci USA 94(22):12024-12029), FTZ-F1 (Yu et al. (1997) Nature 385:552-555; Guichet et al. (1997) Nature 385:548-552), and EGON (Dittrich et al. (1997) Development 124(13):2515-2525).

Embryos injected with double-stranded DHR4 or DSF RNA showed significant embryonic lethality (98% and 85%, respectively). DHR4 and DSF can also be required during larval development as many genes are essential at multiple developmental stages. Injection of double-stranded DERR RNA resulted in lethality predominantly during larval stages (61%). By contrast, injection of double-stranded DFAX1 or DFAX2 RNA showed some lethality during embryonic stages, although not substantially different than buffer-injected control animals.

Lethality resulting from loss of nuclear receptor function is predicted to be mimicked by provision of an antagonist substance that specifically binds a given receptor. Nuclear receptor antagonists can be identified by methods known in the art and as further disclosed in the section entitled Identification of Insect Nuclear Receptor Modulators, herein below. The essentiality of nuclear receptors DHR4, DSF, and DERR, disclosed herein for the first time, identifies the utility of antagonists that block or mitigate the activity of DHR4, DSF, and DERR as insecticides.

III.B. Gain-Of-Function Analyses

Ectopic expression systems have been used to elucidate gene function when classical loss-of-function genetics is not straightforward. For example, heat-induced expression of spaghetti squash, which encodes the nonmuscle myosin II regulatory light chain, can effectively rescue the early lethality of spaghetti squash mutants, facilitating the analysis of phenotypes later in development (Edwards & Kiehart (1996) Development 122:1499). Similarly, dominant phenotypes generated by overexpressing a gene of interest have been used to address post-embryonic gene functions, particularly in cases where gene mutation results in embryonic lethality. See e.g., Lam et al. (1999) Dev Biol 212(1):204-216; Woodard et al. (1994) Cell 79(4):607-615).

Transgenic methods for ectopic expression in Drosophila utilize promoters that drive either constitutive or regulated expression of the gene of interest. Constructs designed for ectopic expression can be prepared in a transformation vector, and are introduced into the fly genome by germ line transformation. A transgenic line is established, and ectopic expression of the gene of interest can be analyzed in a wild type or mutant genetic background.

In one embodiment, a heat shock promoter can be used to temporally regulate gene expression (Lis et al. (1983) Cell 35:403; Struhl (1985) Nature 318:677; Schneuwly et al. (1987) Nature 325:816). Using this approach, the level of ectopic gene expression can be easily modulated by altering the temperature and/or duration of the heat treatment.

Overexpression of nuclear receptors can reveal the role of an activated nuclear receptor. Provision of ligand and/or apo-receptor (a receptor not bound by ligand) favors formation of the liganded receptor. Similarly, provision of excess nuclear receptor overexpression can also lead to an excess of active receptor. See e.g., Tsai et al. (1998) in Wilson et al., eds, Williams Textbook of Endocrinology, pp. 55-94, W.B. Saunders Company, Philadelphia, Pa., and references cited therein. To create this situation in vivo, a nuclear receptor can be overexpressd using a heterologous transgene. This strategy enables a functional assessment of orphan nuclear receptors, wherein a ligand has not yet been identified. A phenotype observed following nuclear receptor overexpression is predicted to also be generated by abnormally elevated levels of endogenous ligand or by administration of a nuclear receptor agonist.

The present invention discloses overexpression of nuclear receptors during Drosophila larval development. Transgenic Drosophila lines were employed that carry heat inducible nuclear receptor transgenes, as described in Example 6. During Drosophila development, the first larval instar begins with hatching of the embryo, and culminates with the first larval molt at approximately 24 hours after hatching (25° C.). Transgenic larvae were briefly heat treated (1 hr at 37° C.) at the beginning of larval development (0-2 hrs +/−15 min) or alternatively at the end of larval development (20-22 hrs +/−15 min), immediately prior to molting. Control experiments omitted heat treatment. The genotype from which the transgenic lines are derived, w¹¹¹⁸, were treated in parallel experiments as an additional control. The developmental progress of larvae was monitored at 24 hours following heat treatment and at puparium formation.

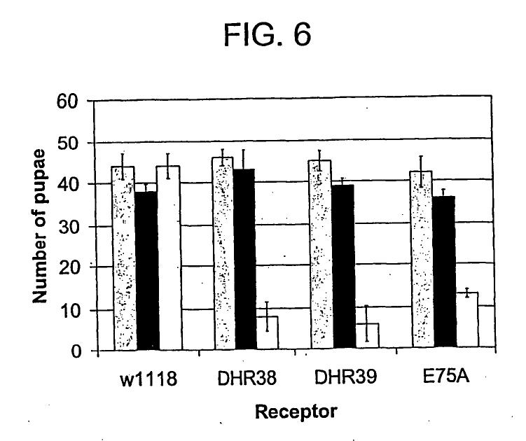

Heat-induced expression of DHR38, DHR39, and E75A at the end of the first instar also resulted in larval lethality (Table 5, FIG. 6). By contrast, transgenic larvae that were not heat-treated were substantially viable, demonstrating that the presence of the transgene, in the absence of induced nuclear receptor expression, is not responsible for the observed lethality. Further, transgenic larvae that were heat treated at the beginning of first instar larval development, w¹¹¹⁸ larvae that were heat treated either at the beginning or end of larval development, and non-heat-treated w¹¹¹⁸ larvae were also substantially viable. Thus, the lethality observed in transgenic larvae that were heat treated just prior to molting (20-22 hr) is attributable to induced nuclear receptor expression, and is not a consequence of heat treatment alone.

TABLE 5
Percent Viability of Heat-Treated Larvae

| | heat treatment | | |
|---|---|---|---|
| larvae | none | 0-2 hr | 20-22 hr |
| control | 88% | 76% | 88% |
| hs-DHR38 | 92% | 86% | 16% |
| hs-DHR39 | 90% | 78% | 12% |
| hs-E75A | 84% | 72% | 26% |

Examination of lethal larvae that were heat treated to induce expression of DHR39 revealed the presence of multiple mouthhooks (28%). This phenotype is consistent with a defect in epidermal molting, whereby mouthhooks are normally expelled (Perrimon et al. (1985) Genetics 11:23-41; Demerec (1965) Biology of Drosophila, Hafner Publishing, New York, N.Y.

Lethality resulting from overexpression of DHR38, DHR39, and E75A is predicted to be mimicked by provision of agonists that bind these receptors. Nuclear receptor agonists can be identified by methods known in the art and as further disclosed in the section entitled Identification of Insect Nuclear Receptor Modulators, herein below. Prior to the disclosure of the subject application, the larval lethal phenotypes conferred by overexpression of DHR38, DHR39, and E75A was unknown. The phenotypic characterization of nuclear receptor modulation during larval development, disclosed herein, identifies the utility of nuclear receptor agonists that activate DHR38, DHR39, E75A, and homologues of the indicated nuclear receptors as insecticides.

Gain-of-function phenotypes of new Drosophila nuclear receptors DERR (SEQ ID NO:2), DFAX1 (SEQ ID NO:6), and DFAX2 (SEQ ID NO:10) disclosed herein can be addressed using ectopic expression techniques in Drosophila that are known in the art. The present invention provides nucleotide sequences (SEQ ID NOs:1, 5, and 9) encoding such receptors that can be used to construct vectors for ectopic expression.

IV. Recombinant Expression of Insect Nuclear Receptors

For recombinant production of a protein of the invention in a host organism, a nucleotide sequence encoding the protein is inserted into an expression cassette designed for the chosen host and introduced into the host where it is recombinantly produced. The choice of the specific regulatory sequences such as promoter, signal sequence, 5′ and 3′ untranslated sequence, and enhancer appropriate for the chosen host is within the level of ordinary skill in the art. The resultant molecule, containing the individual elements linking in the proper reading frame, is inserted into a vector capable of being transformed into the host cell.

Expression constructs can be transfected into a host cell by a standard method suitable for the selected host, including electroporation, calcium phosphate precipitation, DEAE-Dextran transfection, liposome-mediated transfection, infection using a retrovirus, transposon-mediated transfer, and particle bombardment techniques. The expression cassette sequence carried in the expression construct can be stably integrated into the genome of the host or it can be present as an extrachromosomal molecule.

Suitable expression vectors and methods for recombinant production of proteins are known for host organisms such as E coli, yeast, and insect cells. See e.g., Lucknow & Summers (1988) Bio/Technol 6:47. Representative methods for recombinant production of an insect nuclear receptor in E. coli are disclosed in Example 7.

Additional suitable expression vectors are baculovirus expression vectors, e.g., those derived from the genome of Autographica californica nuclear polyhedrosis virus (AcMNPV). A preferred baculovirus/insect system is PVL1392/PVL1393 used to transfect Spodoptera frugiperda (SF9) cells in the presence of linear Autographica californica baculovirus DNA (Pharmingen of San Diego, Calif.). The resulting virus is used to infect HighFive Trichoplusia ni cells (Invitrogen Corporation of Carlsbad, Calif.). Representative methods for recombinant production of an insect nuclear receptor in insect cells are disclosed in Example 8.

Recombinantly produced proteins can be isolated and purified using a variety of standard techniques. The actual techniques used varies depending upon the host organism used, whether the protein is designed for secretion, and other such factors. Such techniques are known to the skilled artisan. See Ausubel et al. (1992) Current Protocols in Molecular Biology, John Wylie and Sons, Inc., New York, N.Y.

The present invention further encompasses recombinant expression of the disclosed insect nuclear receptors, or portion thereof, in plants, as described further herein below under the section entitled Transgenic Plants.

V. Production of Insect Nuclear Receptor Antibodies

In another aspect, the present invention provides a method of producing an antibody immunoreactive with an insect nuclear receptor polypeptide, the method comprising recombinantly or synthetically producing an insect nuclear receptor polypeptide, or portion thereof, to be used as an antigen. The insect nuclear receptor polypeptide is formulated so that it is can be used as an effective immunogen. An animal is immunized with the formulated insect nuclear receptor polypeptide to generate an immune response in the animal. The immune response is characterized by the production of antibodies that can be collected from the blood serum of the animal. Optionally, cells producing an insect nuclear receptor antibody can be fused with myeloma cells, whereby a monoclonal antibody can be selected. Exemplary methods for producing a monoclonal antibody that recognizes an insect nuclear receptor protein are described in Example 4. Preferred embodiments of the method use a polypeptide set forth as any one of SEQ ID NOs:2, 6, 10, 14, 18, 20, 22, 24, and 26.

The present invention also encompasses antibodies and cell lines that produce monoclonal antibodies as described herein.

The foregoing antibodies can be used in methods known in the art relating to the localization and activity of the insect nuclear receptor polypeptide sequences of the invention, e.g., for cloning of insect nuclear receptor nucleic acids, immunopurification of insect nuclear receptor polypeptides, imaging insect nuclear receptor polypeptides in a biological sample, and measuring levels thereof in appropriate biological samples.

VI. Methods for Detecting an Insect Receptor Nucleic Acid

In another aspect of the invention, a method is provided for detecting a nucleic acid molecule that encodes an insect nuclear receptor polypeptide. Such methods can be used to detect insect nuclear receptor gene variants and related resistance gene sequences. The disclosed methods facilitate genotyping, cloning, gene mapping, and gene expression studies.

The nucleic acids of the present invention can be used to clone genes and genomic DNA comprising the disclosed sequences. Alternatively, the nucleic acids of the present invention can be used to clone genes and genomic DNA of related sequences, preferably nuclear receptor genes in pest insects and nematodes. Using the nucleic acid sequences disclosed herein, such methods are known to one skilled in the art. See, for example, Sambrook et al., eds (1989) Molecular Cloning, Cold Spring Harbor Laboratory Press, Cold Spring Harbor, N.Y. Representative methods are also disclosed in Examples 3 and 4. Preferably, the nucleic acids used for this method comprise sequences set forth as any one of SEQ ID NOs:1, 5, 9, 13, 17, 19, 21, 23, and 25.

In one embodiment, genetic assays based on nucleic acid molecules of the present invention can be used to screen for genetic variants by a number of PCR-based techniques, including single-strand conformation polymorphism (SSCP) analysis (Orita et al. (1989) Proc Natl Acad Sci USA 86(8):2766-2770), SSCP/heteroduplex analysis, enzyme mismatch cleavage, direct sequence analysis of amplified exons (Kestila et al. (1998) Mol Cell 1(4):575-582; Yuan et al. (1999) Hum Mutat 14(5):440-446), allele-specific hybridization (Stoneking et al. (1991) Am J Hum Genet 48(2):370-82), and restriction analysis of amplified genomic DNA containing the specific mutation. Automated methods can also be applied to large-scale characterization of single nucleotide polymorphisms (Brookes (1999) Gene 234(2):177-186; Wang et al. (1998) Science 280(5366):1077-1082). Preferred detection methods are non-electrophoretic, including, for example, the TAQMAN™ allelic discrimination assay, PCR-OLA, molecular beacons, padlock probes, and well fluorescence. See Landegren et al. (1998) Genome Res 8:769-776.

VII. Methods for Detecting an Insect Nuclear Receptor Polypeptide

In another aspect of the invention, a method is provided for detecting a level of insect nuclear receptor polypeptide using an antibody that specifically recognizes an insect nuclear receptor polypeptide, or portion thereof. In a preferred embodiment, biological samples from an experimental subject and a control subject are obtained, and insect nuclear receptor polypeptide is detected in each sample by immunochemical reaction with the insect nuclear receptor antibody. More preferably, the antibody recognizes amino acids of any one of SEQ ID NOs:2, 6, 10, 14, 18, 20, 22, 24, and 26; and is prepared according to a method of the present invention for producing such an antibody.

In one embodiment, an insect nuclear receptor antibody is used to screen a biological sample for the presence of an insect nuclear receptor polypeptide. A biological sample to be screened can be a biological fluid such as extracellular or intracellular fluid, or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide. In accordance with a screening assay method, a biological sample is exposed to an antibody immunoreactive with an insect nuclear receptor polypeptide whose presence is being assayed, and the formation of antibody-polypeptide complexes is detected. Techniques for detecting such antibody-antigen conjugates or complexes are known in the art and include but are not limited to centrifugation, affinity chromatography and the like, and binding of a labeled secondary antibody to the antibody-candidate receptor complex.

A modulator that shows specific binding to an insect modulator can also be used to detect an insect nuclear receptor. Representative techniques for assaying specific binding include are described herein above under the heading “Identification of Insect Nuclear Receptor Modulators”.

The disclosed methods for detecting an insect nuclear receptor polypeptide can be useful to determine altered levels of gene expression that are associated with particular conditions such as enhanced tolerance to insecticides that target a particular insect nuclear receptor polypeptide.

VIII. Identification of Nuclear Receptor Modulators

The present invention further discloses a method for identifying a compound that modulates an insect nuclear receptor. As used herein, the terms “candidate substance” and “candidate compound” are used interchangeably and refer to a substance that is believed to interact with another moiety, wherein a biological activity is modulated. For example, a representative candidate compound is believed to interact with an insect nuclear receptor polypeptide, or fragment thereof, and can be subsequently evaluated for such an interaction. Exemplary candidate compounds that can be investigated using the methods of the present invention include, but are not restricted to, viral epitopes, peptides, enzymes, enzyme substrates, co-factors, lectins, sugars, oligonucleotides or nucleic acids, oligosaccharides, proteins, chemical compounds, small molecules, and antibodies. A candidate compound to be tested can be a purified molecule, a homogenous sample, or a mixture of molecules or compounds.

As used herein, the term “modulate” means an increase, decrease, or other alteration of any or all chemical and biological activities or properties of a wild-type insect nuclear receptor polypeptide, preferably an insect nuclear receptor polypeptide of any one of the even-numbered SEQ ID NOs:2-34. Preferably, an insect nuclear receptor modulator is an agonist of an insect nuclear receptor protein activity. As used herein, the term “agonist” means a substance that synergizes or potentiates the biological activity of a functional insect nuclear receptor protein. As used herein, the term “antagonist” refers to a substance that blocks or mitigates the biological activity of an insect nuclear receptor polypeptide.

In accordance with the present invention there is also provided a rapid and high throughput screening method that relies on the methods described above. This screening method comprises separately contacting each compound with a plurality of substantially identical target molecules. In such a screening method the plurality of target molecules preferably comprises more than about 10⁴ samples, or more preferably comprises more than about 5×10⁴ samples. In an alternative high-throughput strategy, each target molecule can be contacted with a plurality of candidate compounds.

The disclosed methods can also be used to identify a modulator that interacts with an insect nuclear receptor ligand binding domain. Such assays can employ a target molecule comprising the full-length nuclear receptor polypeptide. Alternatively, an isolated ligand binding domain can be recombinantly produced for use in the assay. See Coffer et al. (1996) J Steroid Biochem Mol Biol 58:467-477; Tetel et al. (1997) Mol Endocrinol 11:1114-1128; Rochel et al. (1997) Biochem Biophys Res Commun 230:293-296). Optionally, additional sequences, such as receptor, GST, or polyhistidine sequences, can be fused to the amino terminal of the ligand binding domain to stabilize the conformation of the recombinantly expressed ligand binding domain. See e.g., Simental et al. (1991) J Biol Chem 266:510-518; Nemoto et al. (1992) J Steroid Biochem Mol Biol 42:803-812; Cooper et al. (1996) J Steroid Biochem Mol Biol 57:251-257; Eul et al. (1989) EMBO J. 8:83-90; Lin et al. (1991) Mol Endocrinol 5:485-492; Wagner et al. (1995) Nature 378:690-697; Dallery et al. (1993) Biochem 32:12428-12436; Lupisella et al. (1995) J Biol Chem 270:24884-24890; Rochel et al. (1997) Biochem Biophys Res Commun 230:293-296; Leng et al. (1995) Mol Cell Biol 15:255-263.

In one embodiment, the disclosed methods for identifying modulators of insect nuclear receptors are performed using nuclear receptor sequences set forth as any one of even-numbered 2-34. In particular, the loss-of-function larval lethality that is observed when DHR4, DERR, or DSF function is disrupted (FIG. 5) suggests that antagonists of DHR4, DERR, or DSF can be useful as insecticides. The larval lethal phenotype that is observed when DHR38, DHR39, or E75A is overexpressed in Drosophila (FIG. 6) suggest that agonists of DHR38, DHR39, or E75A can be useful as insecticides.

The disclosed methods for identifying modulators of insect nuclear receptors can be performed using nucleic acid sequences derived from a pest organism. The nuclear receptor sequences disclosed herein provide methods for identifying homologous sequences in pest species. Such techniques are well know to those in the art. See for example, Sambrook et al., eds (1989) Molecular Cloning, Cold Spring Harbor Laboratory Press, Cold Spring Harbor, N.Y., and Examples 3 and 4 herein below.

In a preferred embodiment, the disclosed methods for identifying modulators employ a Heliothis βFTZ-F1 (SEQ ID NO:18), Heliothis E75 (SEQ ID NO:20), or Heliothis USP (SEQ ID NO:22) polypeptide. The larval lethal phenotype that is observed when E75A is overexpressed in Drosophila (FIG. 5), suggest that agonists of E75A can be useful as insecticides. Genetic data in Drosophila shows that loss-of-function mutation in any one of USP, βFTZ-F1, or E75A, also confers larval lethality (Yu et al. (1997) Nature 385:552-555; Johnson & Garza (1998) Ann Dros Res Conf 39:430A), suggesting that antagonists of USP, βFTZ-F1, and E75A can also be useful as insecticides.

Representative methods for identification of a substance that binds and thereby modulates an insect nuclear receptor are disclosed herein below. The term “binding” refers to an affinity between two molecules, for example, a ligand and a receptor. As used herein, “binding” means a preferential binding of one molecule for another in a mixture of molecules. The binding of the molecules can be considered specific if the binding affinity is about 1×10⁴ M⁻¹ to about 1×10⁶ M⁻¹ or greater. Binding of two molecules also encompasses a quality or state of mutual action such that an activity of one protein or compound on another protein is inhibitory (in the case of an antagonist) or enhancing (in the case of an agonist). To demonstrate saturable binding of a candidate compound, identified by any such method, to a nuclear receptor ligand binding domain, Scatchard analysis can be carried out as described, for example, by Mak et al. (1989) J Biol Chem 264:21613:21618.

VIII.A. Protein Binding Assays

Several techniques can be used to detect interactions between a protein and a chemical ligand without employing an in vivo ligand. Representative methods include, but are not limited to, Fluorescence Correlation Spectroscopy, Surface-Enhanced Laser Desorption/Ionization Time-Of-flight Spectroscopy, and Biacore technology, as described herein below. These methods are amenable to automated, high-throughput screening.

Fluorescence Correlation Spectroscopy (FCS) measures the average diffusion rate of a fluorescent molecule within a small sample volume (Madge et al. (1972) Phys Rev Lett 29:705-708; Maiti et al. (1997) Proc Natl Acad Sci USA 94:11753-11757). The sample size can be as low as 10³ fluorescent molecules and the sample volume as low as the cytoplasm of a single bacterium. The diffusion rate is a function of the mass of the molecule and decreases as the mass increases. FCS can therefore be applied to polypeptide-ligand interaction analysis by measuring the change in mass and therefore in diffusion rate of a molecule upon binding. In a typical experiment, the target to be analyzed is expressed as a recombinant polypeptide with a sequence tag, such as a poly-histidine sequence, inserted at the N-terminus or C-terminus. The expression takes place in E coli, yeast or mammalian cells. The polypeptide is purified using chromatographic methods. For example, the poly-histidine tag can be used to bind the expressed polypeptide to a metal chelate column such as Ni²⁺ chelated on iminodiacetic acid agarose. The polypeptide is then labeled with a fluorescent tag such as carboxytetramethylrhodamine or BODIPY™ (Molecular Probes of Eugene, Oreg.). The polypeptide is then exposed in solution to the potential ligand, and its diffusion rate is determined by FCS using instrumentation available from Carl Zeiss, Inc. (Thornwood, New York). Ligand binding is determined by changes in the diffusion rate of the polypeptide.

Surface-Enhanced Laser Desorption/Ionization (SELDI) was developed by Hutchens & Yip (1993) Rapid Commun Mass Spectrom 7:576-580. When coupled to a time-of-flight mass spectrometer (TOF), SELDI provides a technique to rapidly analyze molecules retained on a chip. It can be applied to ligand-protein interaction analysis by covalently binding the target protein, or portion thereof, on the chip and analyzing by MS the small molecules that bind to this protein (Worrall et al. (1998) Anal Biochem 70:750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system able to pipet the ligands in a sequential manner (autosampler). The chip is then washed in solutions of increasing stringency, for example a series of washes with buffer solutions containing an increasing ionic strength. After each wash, the bound material is analyzed by submitting the chip to SELDI-TOF. Ligands that specifically bind the target are identified by the stringency of the wash needed to elute them.

Biacore relies on changes in the refractive index at the surface layer upon binding of a ligand to a target polypeptide immobilized on the layer. In this system, a collection of small ligands is injected sequentially in a 2-5 microliter cell, wherein the target polypeptide is immobilized within the cell. Binding is detected by surface plasmon resonance (SPR) by recording laser light refracting from the surface. In general, the refractive index change for a given change of mass concentration at the surface layer is practically the same for all proteins and peptides, allowing a single method to be applicable for any protein (Liedberg et al. (1983) Sensors Actuators 4:299-304; Malmquist (1993) Nature 361:186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics of on rate and off rate allows the discrimination between non-specific and specific interaction.

VIII.B. Peptide Interaction Assays

Methods for displaying diverse peptide libraries enable rapid library construction, amplification, and selection of ligands directed against a target molecule. See Lowman (1997) Annu Rev Biophys Biomol Struct 26:401-424; Sidhu (2000) Curr Opin Biotech 11(6):610-616; and U.S. Pat. No. 5,510,240. Assays can also be employed that select peptides capable of disrupting the interaction between a nuclear receptor and a requisite co-factor, as described by Hall et al. (2000) Mol Enodcrinol 14(12):2010-2023; Northrop et al. (2000) Mol Endocrinol 14(5)605-622; International Publication No. WO 00/37077, herein incorporated by reference.

VIII.C. Transcriptional Assays

The present invention also provides methods for identifying modulators of insect nuclear receptor transcriptional activation. One strategy employs an expression system comprising: (1) an insect nuclear receptor comprising a functional ligand binding domain of an insect nuclear receptor, (2) a target gene expression cassette comprising a response element regulated by the chimeric nuclear receptor operatively linked to a reporter gene, and (3) a test compound. Methods for constructing a chimeric nuclear receptor gene and a target gene expression cassette are described herein below under the heading “Chimeric Receptors for Inducible Gene Expression”. See also, Wentworth et al. (2000) J Endocrinol 166(3):R11-16; Yang & Chen (1999) Cancer Res 59(18):4519-4524, and U.S. Pat. No. 4,981,784, herein incorporated by reference.

The term “reporter gene” refers to a heterologous gene encoding a product that is readily observed and/or quantitated. A reporter gene is heterologous in that it originates from a source foreign to an intended host cell or, if from the same source, is modified from its original form. Any suitable reporter and detection method can be used in accordance with the disclosed methods. Non-limiting examples of detectable reporter genes that can be operatively linked to a transcriptional regulatory region can be found in Alam and Cook (1990) Anal Biochem 188:245-254 and International Publication No. WO 97/47763. Preferred reporter genes for transcriptional analyses include the lacZ gene (See e.g., Rose and Botstein (1983) Meth Enzymol 101:167-180), Green Fluorescent Protein (GFP) (Cubitt et al. (1995) Trends Biochem Sci 20:448-455), luciferase, or chloramphenicol acetyl transferase (CAT).

An amount of reporter gene can be assayed by any method for qualitatively, or preferably quantitatively, determining presence or activity of the reporter gene product. The amount of reporter gene expression directed by each test substance is compared to an amount of reporter gene expression in the absence of a test substance. A test substance is identified as having agonist activity when there is significant increase in a level of reporter gene expression in the presence of the substance when compared to a level of reporter gene expression in the absence of the test substance. The term “significant increase”, as used herein, refers to an quantified change in a measurable quality that is larger than the margin of error inherent in the measurement technique, preferably an increase by about 2-fold or greater relative to a control measurement, more preferably an increase by about 5-fold or greater, and most preferably an increase by about 10-fold or greater.

VIII.D. Rational Design

The knowledge of the structure a native nuclear receptor polypeptide provides an approach for rational pesticide design. See e.g. Schapira et al. (2000) Proc Natl Acad Sci USA 97(3):1008-1013. The structure of a nuclear receptor polypeptide can be determined by X-ray crystallography or by computational algorithms that generate three-dimensional representations. See Huang et al. (2000) Pac Symp Biocomput 230-41; Saqi et al. (1999) Bioinformatics 15:521-522; International Publication No. WO 99/26966, herein incorporated by reference. Alternatively, a working model of a nuclear receptor structure can be derived by homology modeling (Maalouf et al. (1998) J Biomol Struc Dynamics 15(5):841-851). Computer models can further predict binding of a protein structure to various substrate molecules, which can be synthesized and tested. Additional compound design techniques are described in U.S. Pat. Nos. 5,834,228 and 5,872,011.

IX. Methods for Pest Control

Another aspect of the present invention is a method for pest control by modulation of insect nuclear receptor biological activity. Substances having such activity can be discovered by the methods disclosed herein and include, but are not limited to, chemical compounds, antibodies, and gene products encoded by plant transgenes.

The present invention provides methods for preventing the onset or progression of a pest infestation in a plant. The method comprises administering a modulator of a nuclear receptor set forth as any one of the even-numbered SEQ ID NOs:2-34, wherein modulation of the nuclear receptor results in organismal lethality. Preferably, the lethality occurs during larval development.

IX.A. Formulation

An insect nuclear receptor modulator of the present invention is typically formulated using acceptable vehicles, adjuvants, and carriers as desired. Representative formulations include emulsifiable concentrates, water-miscible liquids, wettable powders, water-soluble powders, oil solutions, flowable powders, aerosols, vapors, granulars, microcapsules, fumigants, ultra-low volume concentrates, fogging concentrates, vapors, impregnating materials, poison baits, and seed dressings. See e.g., Perry et al. (1997) Insecticides in Agriculture and Environment: Retrospects and Prospects, pp. 7-10, Springer-Verlag, New York, N.Y. A formulation can be further selected based on its ability to improve insecticide properties such as storage, handling, application, effectiveness, safety to the applicator and the environment, and cost.

An insecticide formulation can further include a synergist that can enhance the activity of an insect nuclear receptor modulator of the present invention. See Yamamoto (1973) in Casida, ed, Pyrethrum, The Natural Insecticide, pp. 191-170, Academic Press, New York, N.Y.; Hodgson & Tate (1976) in Wilkinson, ed, Insecticide Biochemistry and Physiology, pp. 115-148, Plenum Press, New York, N.Y.; Wilkinson (1976a) in Tahori, ed, Proc 2^{nd} Int Congr on Pesticides and Chemistry, Vol. 2, pp. 117-159, Gordon & Breach, New York, N.Y.; Wilkinson (1976b) in Metcalf & McKelvey, eds, The Future for Insecticides: Needs and Prospects, Vol. 6, pp. 191-178, Wiley, New York, N.Y.; Casida & Quistad (1995) in Casida & Quistad, eds, Pyrethrum Flowers: Production, Chemistry, Toxicology, and Uses, pp. 258-276, Oxford University Press, New York, N.Y. Alternatively, synergism can be accomplished by treatment of a plant prior to application of an insect nuclear receptor modulator, or by application of a synergist at sites on a plant distinct form sites of application of an insect nuclear receptor modulator.

IX.B. In Vivo Assays

The insecticidal activity of a modulator of an insect nuclear receptor can be tested using standard techniques in the art, including topical application, injection, dipping, contact or residual exposure, and feeding/drinking. See e.g., Perry et al. (1997) Insecticides in Agriculture and Environment: Retrospects and Prospects, pp. 12-13, Springer-Verlag, New York, N.Y. As one example, a formulation comprising a modulator is sprayed on a plant, insect larvae are then applied to the plant, and after an appropriate temporal duration, a degree of plant destruction by the larvae is quantitated.

IX.C. Dose and Administration

The toxicity of an insecticide to an organism can be expressed in terms of the amount of compound per unit weight of the organism required to kill 50% of the test population, also referred to as the lethal dose (LD₅₀). The LD₅₀ is usually expressed in milligrams per kilogram of body weight or micrograms per insect. The lethal concentration (LC₅₀) is the concentration of a compound in an external medium that is required to kill 50% of the test population, and is expressed as the percentage or parts per million (ppm) of the active ingredient (Al) in the medium. This value can be used when the exact dose administered to an insect cannot be determined. The effectiveness of a candidate insecticidal substance can also be assayed in terms of lethal time (LC₅₀). LC₅₀ represents the time required to elicit 50% mortality of the test organisms at a specified dose or concentration and is a suitable measure for field tests.

In some instances, a rate of knockdown rather than lethality is measured as a criterion of effectiveness. In such cases the knockdown dose (KD₅₀) or the knockdown time (KT₅₀) can be used to express insecticidal activity.

The present invention also envisions the identification of insecticidal substances wherein killing or knockdown does not constitute the desired criterion. For example, useful assays can also assess non-lethal measures such as, for example, progression to developmental stages, fecundity, egg viability, attractant or repellant activity, paralysis, and anti-feeding activity.

Insect nuclear receptor modulators identified in accordance with methods of the present invention are useful for preventing or treating an insect infestation, and in some cases a nematode infestation, in a plant or animal. Prevention and treatment methods employ an effective amount of the modulator. The term “effective amount” as used herein refers to an amount effective to prevent or ameliorate infestation.

An effective amount can comprise a range of amounts. One skilled in the art can readily assess the potency and efficacy of an insect nuclear receptor modulator of the present invention and adjust the administration regimen accordingly. A modulator of insect nuclear receptor biological activity can be evaluated by a variety of techniques, for example, by using a responsive reporter gene in an transcriptional assay, by assaying interaction of insect nuclear receptor polypeptides with a monoclonal antibody, or by assaying insect viability when a modulator is administered to an insect, each technique described herein. One of ordinary skill in the art can tailor the dosages to a particular application, taking into account the particular formulation and method of administration to be used with the composition as well as the type of plant or animal, the development stage of the plant or animal, and the severity of the infestation to be treated.

IX.D. Transgenic Plants

The present invention also encompasses methods for pest control wherein an insect nuclear receptor modulator is expressed in a plant. Preferably, a nucleic acid, peptide or polypeptide encoded by a transgene in a plant modulates the activity of any of SEQ ID NOs:1-34. In one embodiment, a transgene can encode a peptide that specifically binds an insect nuclear receptor of the present invention. In another embodiment, a construct encoding an antibody that specifically binds an insect nuclear receptor of the present invention can be expressed in plants to confer insect control. See e.g., U.S. Pat. No. 5,686,600, the contents of which are herein fully incorporated by reference. Methods for generating a transgenic plant are known in the art and are discussed further herein below.

IX.E. Target Organisms

Insect nuclear receptor modulators discovered according to the methods disclosed herein can be used for the prevention or amelioration of a pest infestation. The term “pest” as used herein refers to any organism that damages a plant, including mature plants, seedlings, and stored grain. The term “pest” also refers to any organism that causes disease in an animal. The compositions and methods disclosed herein are envisioned to be particularly useful to prevent or to treat infestation of insect pests, including but not limited to aphids, locusts, spider mites, boll weevils, and pests that attack stored grains (e.g., Tribolium and Tenebrio). The present disclosure is also relevant to methods for controlling soil nematodes and plant-parasitic nematodes such as Melooidogyne.

X. Chimeric Receptors for Inducible Gene Expression

Transgenic methods have enabled the generation of plants with improved traits by expression of a transgene encoding a heterologous polypeptide of interest. Ideally, the temporal profile of transgene expression can be controlled.

The present invention envisions a gene switch method that employs three or more components: (1) a nuclear receptor expression cassette, (2) a ligand that binds the polypeptide encoded by the nuclear receptor expression cassette, and (3) a target gene expression cassette that is modulated in the presence of the encoded nuclear receptor polypeptide further bound by a ligand.

A nuclear expression receptor cassette of the present invention encodes a nuclear receptor polypeptide. In one embodiment, the nuclear receptor polypeptide is composed of a hinge region, a ligand binding domain, a DNA binding domain, and a transactivation domain. The DNA binding domain enables binding of the nuclear receptor polypeptide to a sequence-specific response element in the 5′ regulatory region of a target expression cassette. The hinge domain of the receptor polypeptide resides between the DNA binding and ligand binding domains and influences the activity of the ligand binding domain. The ligand binding domain of the receptor polypeptide can bind a chemical ligand, thereby eliciting a conformational change in the receptor polypeptide that allows the transactivation domain to affect transcription of the target nucleotide sequence.

A “target expression cassette” comprises a nucleotide sequence for a 5′ regulatory region operatively linked to a target nucleotide sequence, the expression of which is activated by a receptor polypeptide in the presence of a chemical ligand. The 5′ regulatory region of the target gene comprises a core promoter sequence, an initiation of transcription sequence, and one or more sequence-specific response elements required for receptor binding to the target gene regulatory region. The promoter sequence can be a minimal promoter. The target expression cassette can also possess a 3′ termination region (stop codon and polyadenylation sequence). The target nucleotide sequence can encode, for example, a polypeptide, an antisense RNA, or a double-stranded RNA molecule. A target sequence can be part of a target cassette transformed into a host organism, or it can be a target sequence of a native host organism gene.

The chimeric receptor polypeptides used in the present invention can have one or more domains obtained from a heterologous source. The use of chimeric receptor polypeptides has the benefit of combining domains from different sources, thus providing a receptor polypeptide activated by a choice of chemical ligands and possessing desirable ligand binding, DNA binding and transactivation characteristics.

It is also considered a part of the present invention that the transactivation (A/B), ligand-binding (E), and DNA-binding (C) domains can be assembled in the chimeric receptor polypeptide in any functional arrangement. For example, where one subdomain of a transactivation domain is found at the N-terminal portion of a naturally-occurring receptor, a chimeric receptor polypeptide of the present invention can include a transactivation domain at the C-terminus in place of, or in addition to, a transactivation domain at the N-terminus. Chimeric receptor polypeptides as disclosed herein can also have multiple domains of the same type, for example, more than one transactivation domain per receptor polypeptide.

X.A. DNA Binding Domain of a Receptor Expression Cassette

A chimeric receptor of the present invention can comprise a DNA-binding domain from any one of SEQ ID NOs:2, 6, 10, 14, 18, 20, 22, 24, and 26. The term “DNA binding domain” as used in the context of a chimeric receptor of the present invention comprises a functional domain that shows high affinity sequence-specific DNA binding. A functional DNA binding domain will generally include the core DNA binding domain, and optionally, sequences adjacent to the core DNA binding domain that contribute to high affinity specific-specific DNA binding.

A gene switch receptor cassette preferably encodes a chimeric nuclear receptor that modulates gene expression as a monomer or dimer. Drosophila βFTZ-F1 and the vertebrate homologue of βFTZ-F1, SF-1, can bind to an extended half-site response element as a monomer and can further function as a transcriptional activator in this context (Ueda et al. (1992) Mol Cell Biol 12(12):5667-5672; Wilson et al. (1993) Mol Cell Biol 13(9):5794-5804). Similarly, a vertebrate homologue of E75, RevErb, represses transcription on both monomeric and dimeric binding sites (Harding & Lazar (1995) Mol Cell Biol 15(11):6479; Lazar & Harding (1998) in Freedman, ed, Molecular Biology of Steroid and Nuclear Hormone Receptors, pp. 269-270, Birkhäuser, Boston, Mass.). The Drosophila DNA-binding domain and C-terminated extension of the core DNA binding domain is highly homologous to RevErb and shows similar DNA-binding properties (Segraves & Hogness (1990) Genes Dev 4:204-219; Lazar & Harding (1998) in Freedman, ed, Molecular Biology of Steroid and Nuclear Hormone Receptors, pp. 270, Birkhäuser, Boston, Mass.).

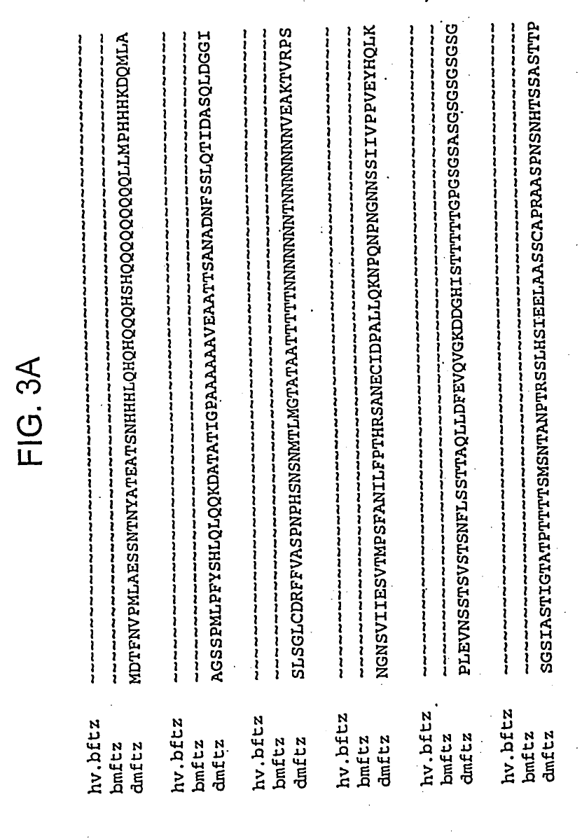

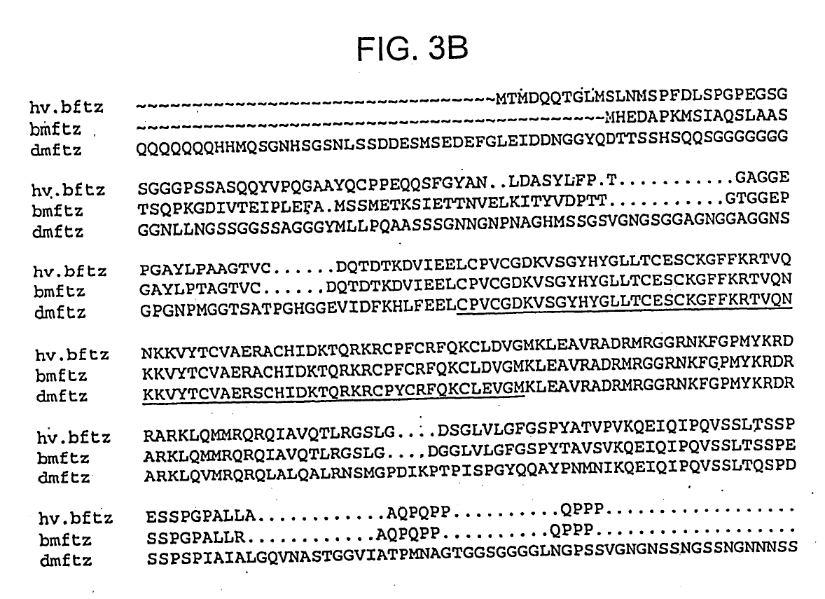

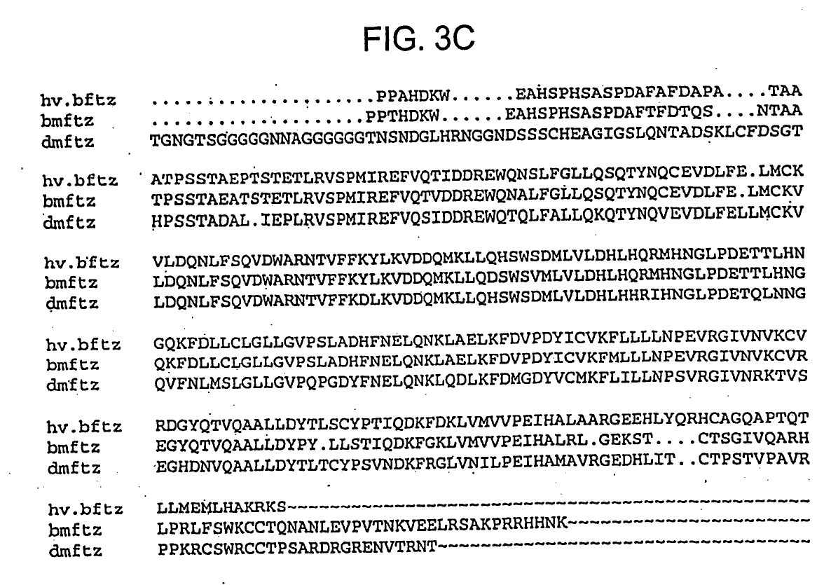

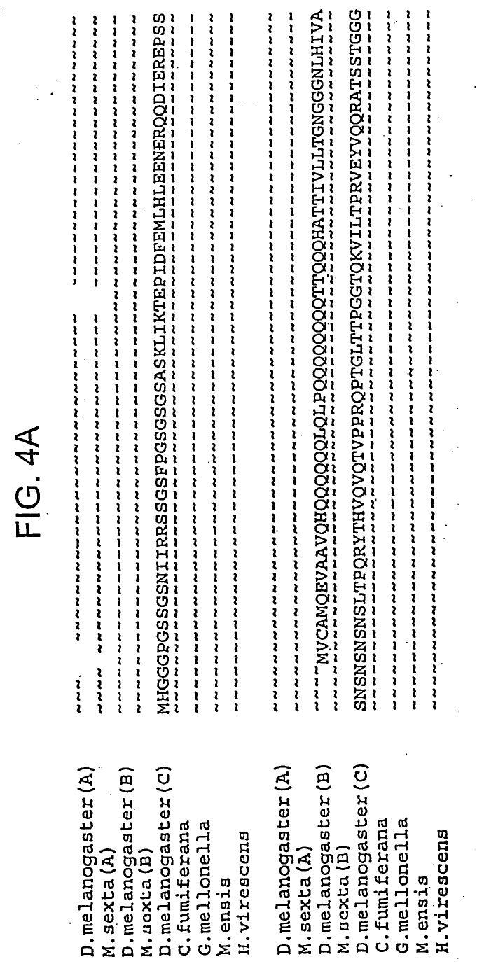

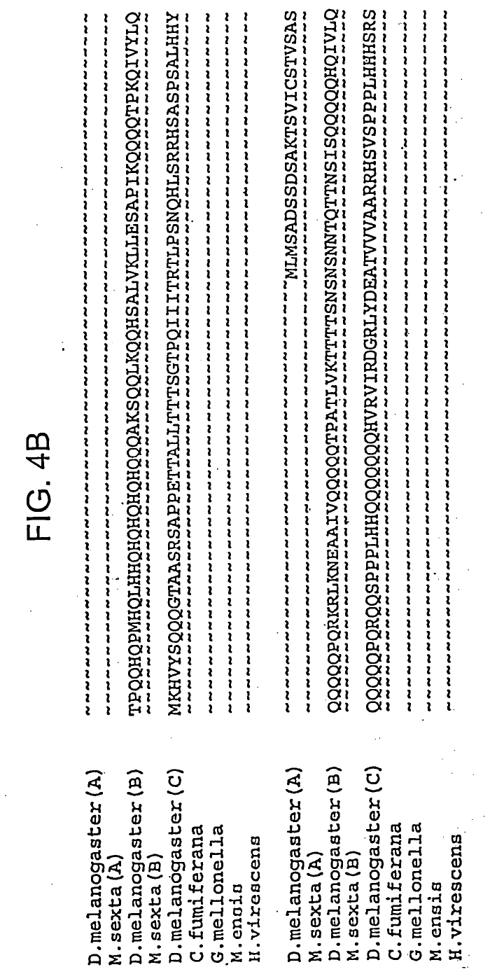

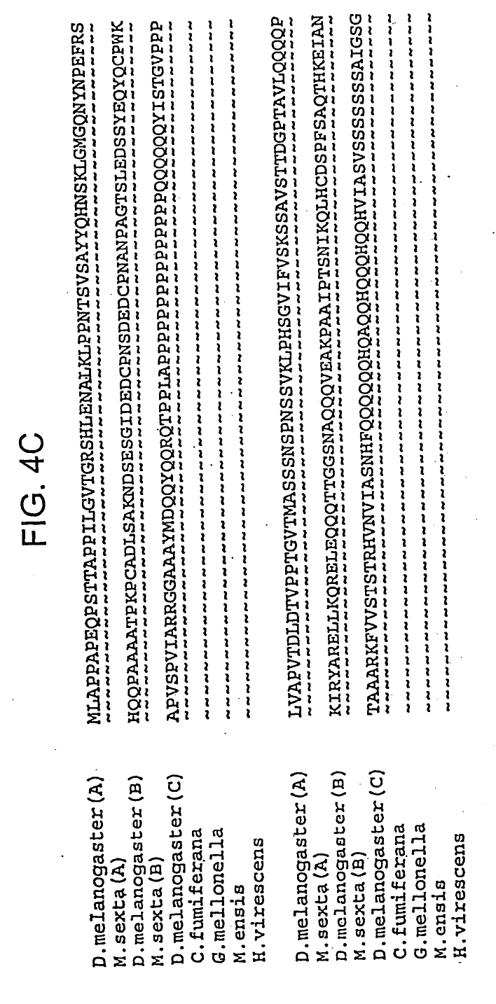

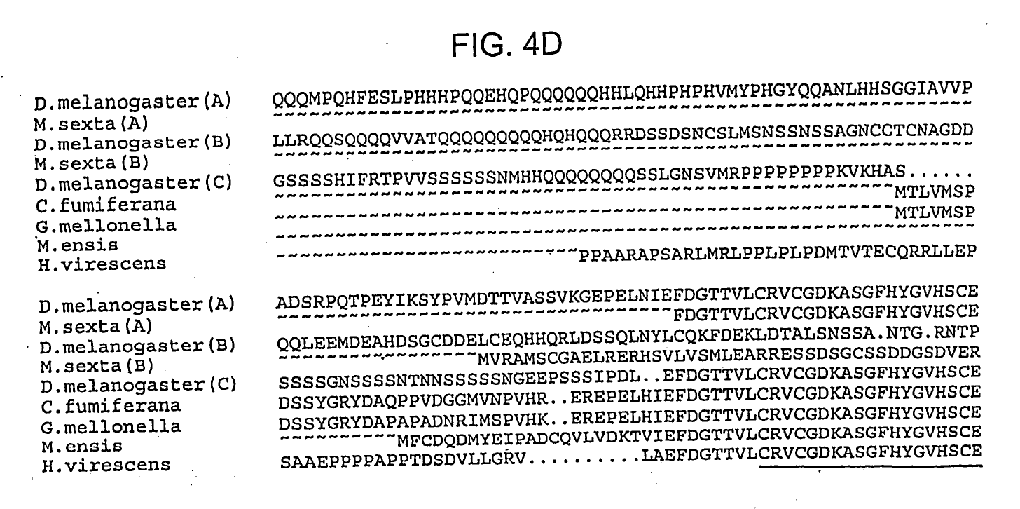

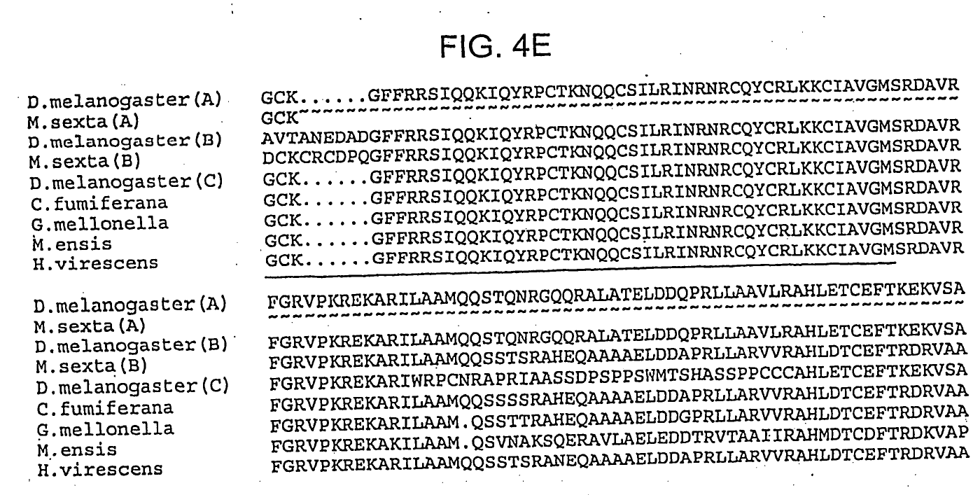

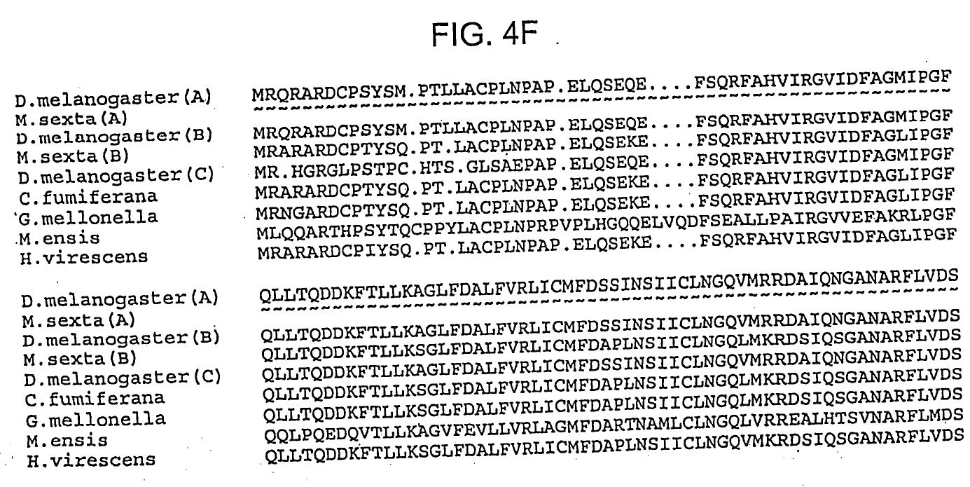

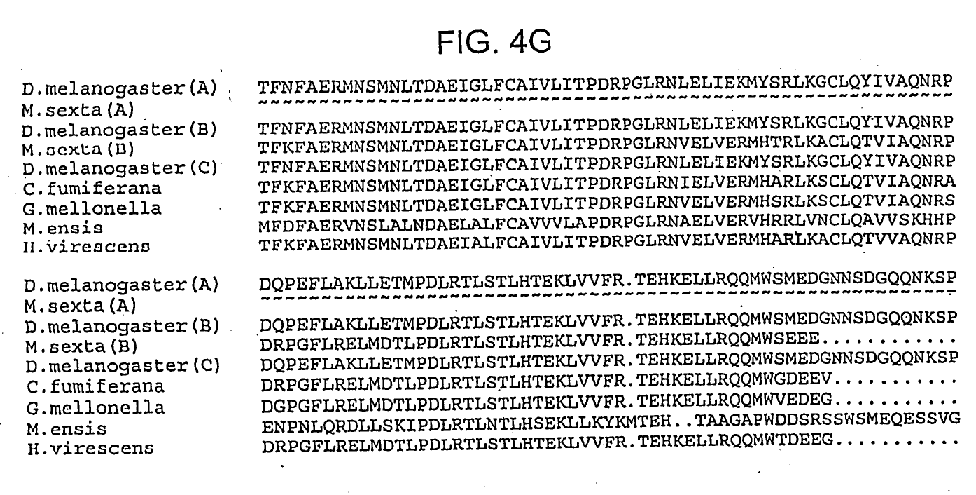

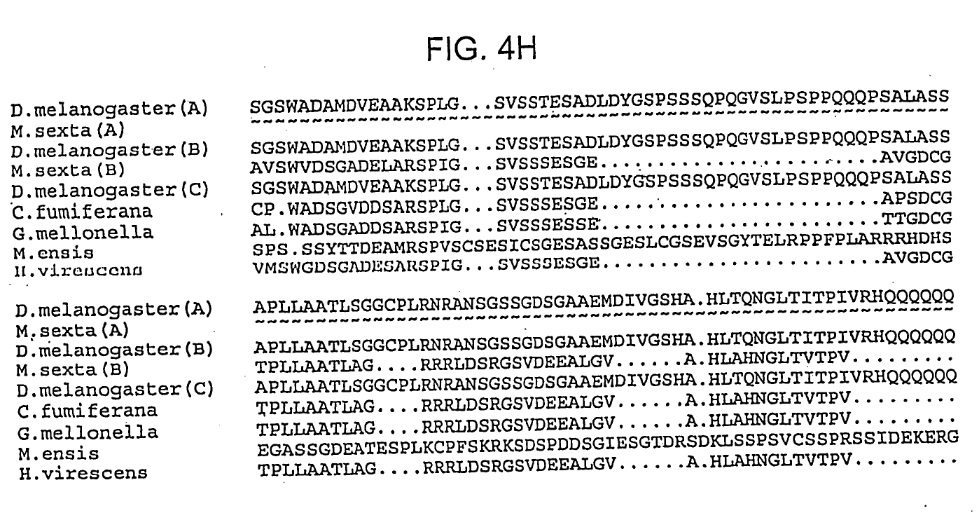

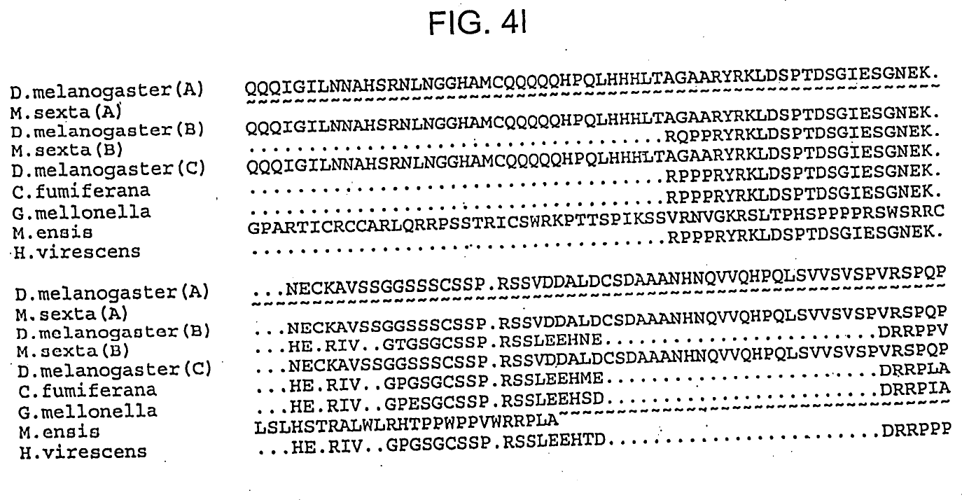

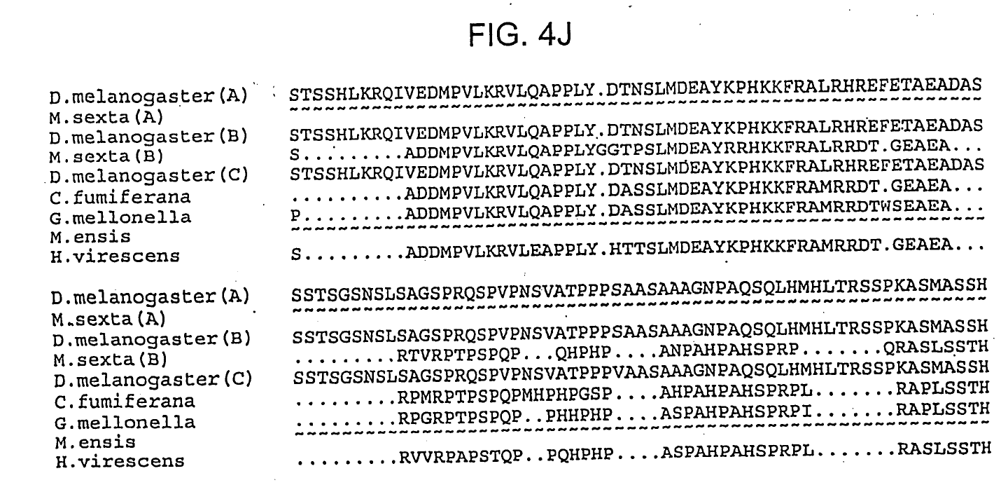

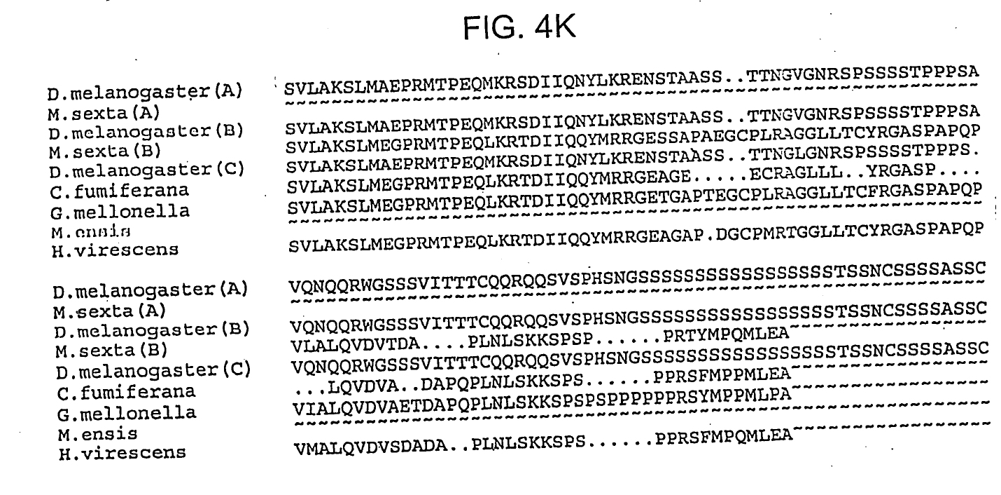

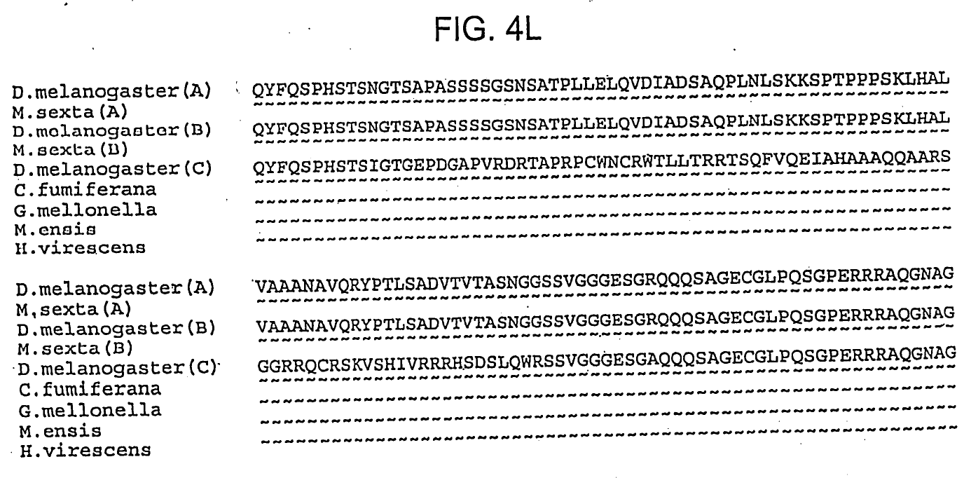

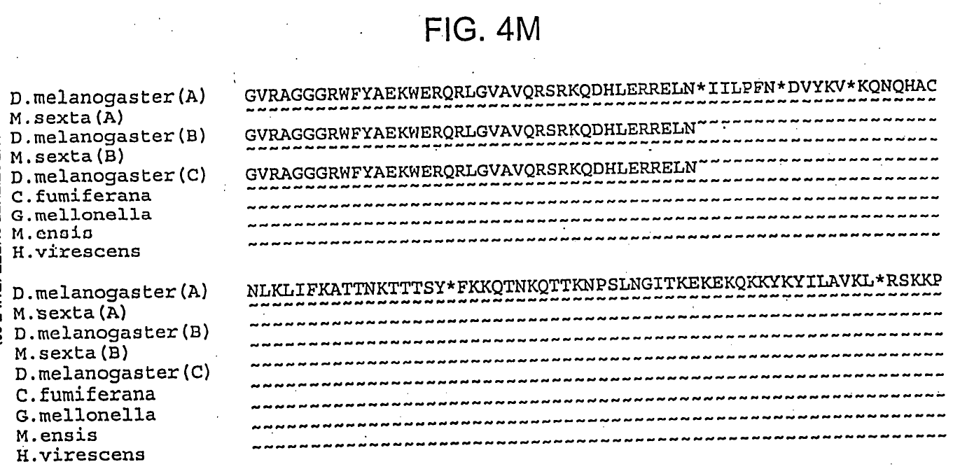

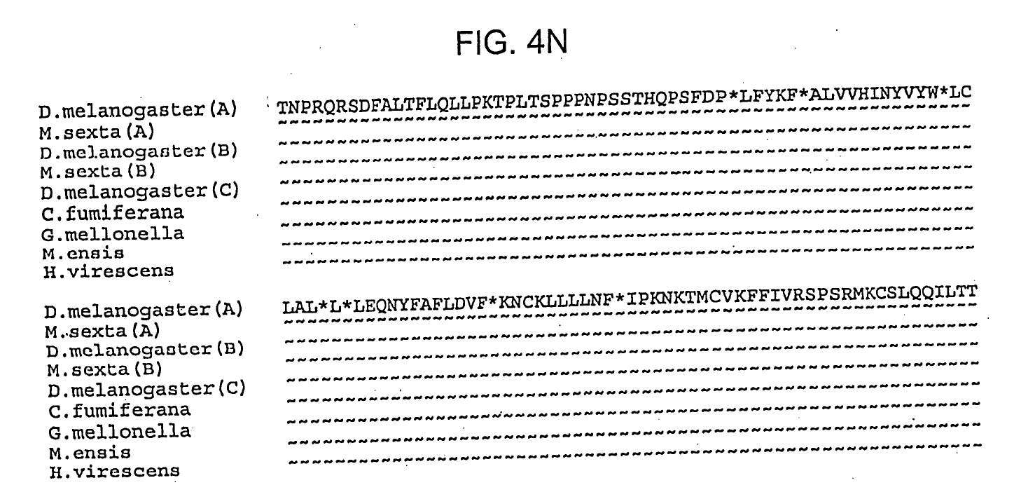

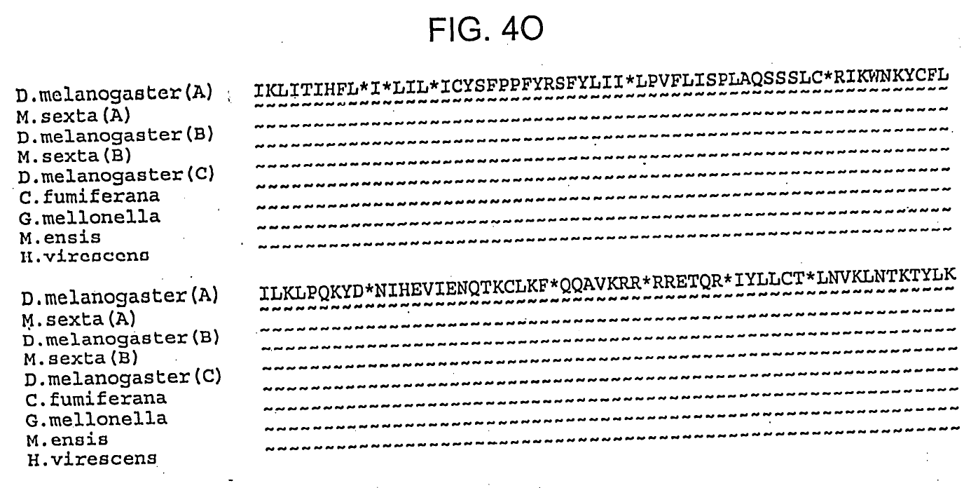

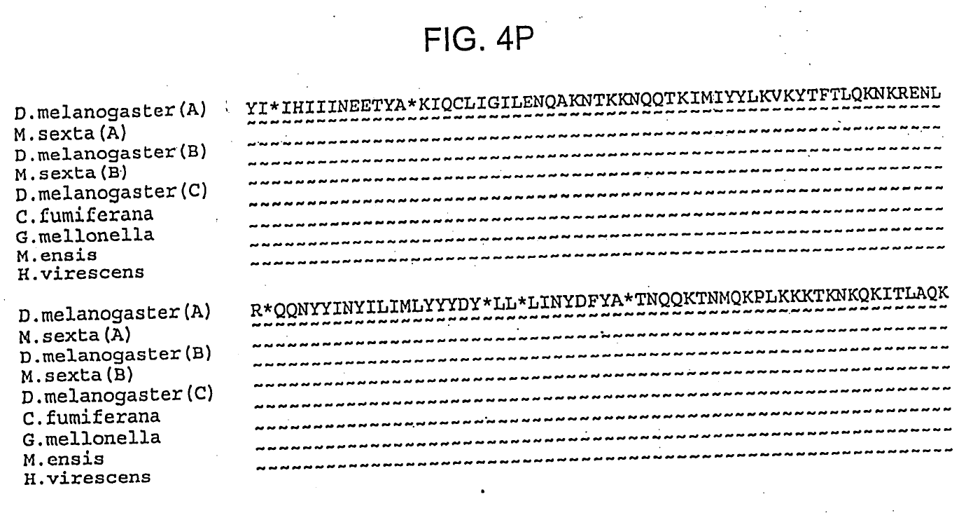

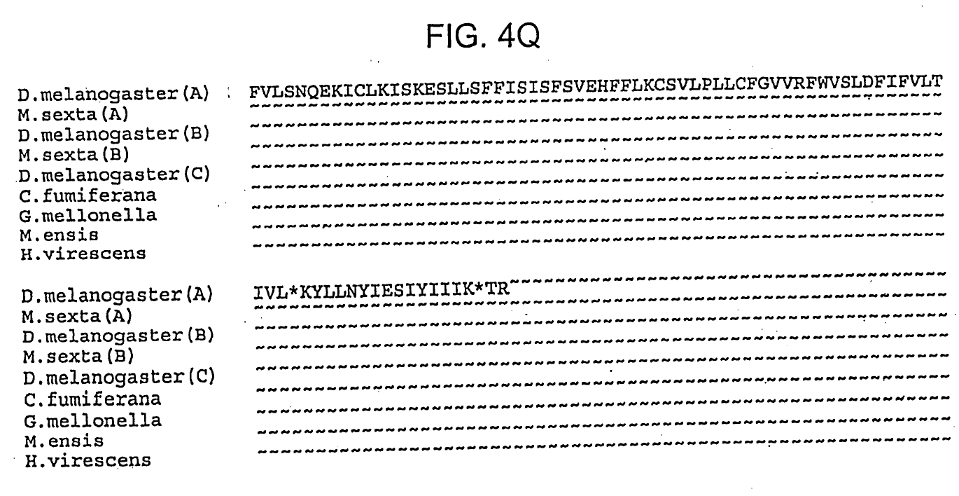

The Heliothis βFTZ-F1 and E75A nuclear receptors disclosed herein are highly conserved compared to their Drosophila and vertebrate homologues (FIGS. 3 and 4). In particular, the DNA binding domain and the C-terminal extension of the core DNA-binding domain of Heliothis βFTZ-F1 (SEQ ID NO:18) is substantially identical to Drosophila βFTZ-F1. The DNA binding domain and the C-terminal extension of the core DNA-binding domain of Heliothis E75 (SEQ ID NO:20) is substantially identical compared to Drosophila E75A. Given the similar DNA binding properties between Drosophila βFTZ-F1 and vertebrate SF-1, and between Drosophila E75A and vertebrate RevErb, Heliothis βFTZ-F1 and E75 likely function as monomeric and/or dimeric transcriptional regulators as well. Thus, the DNA binding domains, optionally with the extended C-terminal DNA binding sequence, can be useful in gene switch receptor expression cassettes that operate as monomeric transcriptional regulators.

The vertebrate nuclear receptor most closely related to DERR, estrogen-related receptor, also binds and activates transcription as a monomer, again utilizing an extended C-terminal DNA binding sequence (Bonnelye et al. (1997) Mol Endocrinol 11 (7):905-916; Johnston et al. (1997) Mol Endocrinol 11 (3):342-352). Thus, the DERR DNA binding domain, optionally also including the C-terminal extended DNA binding sequence, can be useful in gene switch receptor expression cassettes.

Additional flexibility in controlling gene expression by the present invention can be obtained by using DNA binding domains and response elements from other transcriptional activators, which include but are not limited to the bacterial LexA or yeast GAL4 proteins (Brent & Ptashne (1985) Cell 43:729-736i; Sadowski et al. (1988) Nature 335:563-564).

An additional degree of flexibility in controlling gene expression can be obtained by using synthetic DNA binding domains and response elements. Protein engineering experiments can rationally alter the DNA binding characteristics of zinc finger domains to bind to a DNA target sequence of choice (Liu et al. (1997) Proc Natl Acad Sci USA 94:5525-5530; Desjarlais & Berg (1993) Proc Natl Acad Sci USA 90:2252-1860). For example, the use of a synthetic zinc finger binding domain allows the chimeric receptor polypeptide to recognize a target sequence of choice.

A chimeric receptor expression cassette of the present invention comprises a suitable promoter operatively linked to the coding sequence intended for expression. The expression of the nucleotide sequence in the expression cassette can be under the control of a constitutive promoter, an inducible promoter, or a tissue-specific promoter. Depending upon the host cell system utilized, any one of a number of suitable promoters can be used. Promoter selection can be based on expression profile and expression level.

X.B. Target Gene Expression Cassette

A target gene expression cassette includes a response element that is operatively linked to a target gene of interest.

In one embodiment, gene switch systems of the present invention can employ a chimeric receptor expression polypeptide having a DNA binding domain that binds a well-characterized response element, for example, the LexA and GAL4 binding sites (Brent & Ptashne (1985) Cell 43:729-736; Sadowski et al. (1988) Nature 335:563-564).

In another embodiment, a gene switch system of the present invention can employ a chimeric receptor polypeptide comprising a DNA binding domain derived from a novel insect nuclear receptor disclosed herein (SEQ ID NOs:2, 6, 10, 14, 18, 20, 22, 24, and 26). Response elements that are recognized by DNA binding domains of novel nuclear receptors can be determined according to standard methods. Briefly, in vivo footprinting assays can demonstrate protection of DNA sequences from chemical and enzymatic modification within living or permeabilized cells. Similarly, in vitro footprinting assays can protect DNA sequences from chemical or enzymatic modification using protein extracts. Nitrocellulose filter-binding assays and gel electrophoresis mobility shift assays (EMSAs) can track the presence of radiolabeled regulatory DNA elements based on provision of candidate transcription factors. Computer analysis programs, for example TFSEARCH version 1.3 (Yutaka Akiyama: “TFSEARCH: Searching Transcription Factor Binding Sites”, http://www.rwcp.or.jp/papia/), can be used to locate consensus sequences of known cis-regulatory elements within a genomic region.

In a preferred embodiment of the invention, multiple copies of the appropriate response element are placed in the 5′ regulatory region, which allows multiple sites for binding of receptor polypeptide resulting in a greater degree of activation.

X.C. Ligand Binding Domain of a Receptor Expression Cassette

Preferably, the ligand binding domain of a chimeric receptor of the present invention comprises the “E” domain of any one of SEQ ID NOs:2, 6, 10, 14, 18, 20, 22, 24, and 26. For the purpose of creating a receptor expression cassette, the “E” domain is defined operationally as a sequence that is sufficient for ligand binding and can be determined by methods known in the art. A ligand binding domain of an insect nuclear receptor disclosed herein can further be modified to permit and/or optimize binding to a selected ligand. Such modifications can include point mutations and truncation.

X.D. Activation/Repression Domain of the Receptor Expression Cassette

Transactivation (A/B) domains can be defined as amino acid sequences that, when combined with the DNA binding domain in a receptor polypeptide, increase productive transcription initiation by RNA polymerases. See, generally, Ptashne (1988) Nature 335:683-689; Meshi (1995) Plant Cell Physiol 36:1405-1420. Different transactivation domains are known to have different degrees of effectiveness in their abilities to increase transcription initiation. In the present invention, it is desirable to use transactivation domains that have superior transactivating effectiveness in host cells in order to create a high level of target expression cassette expression in response to the presence of chemical ligand. Representative transactivation domains include but are not limited to herpes simplex virus VP16 (Triezenberg et al. (1988) Genes Dev 2(6):718-729), maize C1 (Goff et al. (1991) Genes and Dev 5:298-309), Arabidopsis AP1, and maize Dof1.

As described above, the method of the present invention can be used to increase gene expression over a minimal, basal level. One of the outstanding benefits of the present method, however, is that it can also be used for decreasing or inhibiting gene expression, e.g., gene repression. Controlling gene expression through repression can be accomplished using a repression domain in place of the transactivation domain. Repression domains can be defined as amino acid sequences that, when combined with the DNA binding domain in a receptor polypeptide, decrease the productive transcription initiation by RNA polymerases (Ng (2000) Trends Biochem Sci 25:121-126). Repression domains that can be used with the present invention to decrease expression of a target cassette include but are not limited to the repression domains of AtHD2A (Wu (2000) Plant J 22:19-27), Oshox1, and Oshox3 (Meijer (2000) Mol Gen Genet 263:12-21).

X.E. Additional Features of Gene Switch Expression Cassettes

The transgenic expression of genes can also involve modification of the relevant transgenes to achieve and optimize their expression in a selected host. Modifications can include but are not limited to cloning of open reading frames normally encoded by a single gene in separate expression cassettes, utilization of host codon preferences, adjustment of GC/AT content, inclusion of sequences adjacent to the initiating methionine codon that promote efficient translation, and removal of illegitimate splice sites.

An expression cassette can also comprise any additional sequences required or selected for the expression of the transgene. Such sequences include, but are not limited to, transcription terminators, introns, sequences that can enhance gene expression, and sequences that mediate intracellular targeting of the gene product.

X.F. Ligands for Inducible Gene Expression

A ligand that activates a chimeric receptor polypeptide and that can be used in a gene switch expression system can be identified, for example, using methods described herein above under the heading “Identification of Insect Nuclear Receptor Modulators”.

XI. Transgenic Plants

The present invention envisions expression of insect nuclear receptor modulators and components of nuclear receptor gene switch expression systems in plants. Representative techniques for transforming dicotyledonous and monocotyledonous plants are described herein below.

The phrase “a plant, or parts thereof”, as used herein shall mean an entire plant; and shall mean the individual parts thereof, including but not limited to seeds, leaves, stems, and roots, as well as plant tissue cultures. Transgenic plants of the present invention are understood to encompass not only the end product of a transformation method, but also transgenic progeny thereof.

Representative plants that can be used in transgenic methods disclosed herein include but are not limited to rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, tobacco, tomato, sorghum and sugarcane.

XI.A. Promoters

For in vivo production of an insect nuclear receptor modulator or a chimeric receptor expression cassette in plants, exemplary constitutive promoters are derived from the CaMV 35S, rice actin, and maize ubiquitin genes. See Binet et al. (1991) Plant Sci 79:87-94, Christensen et al. (1989) Plant Mol Biol 12:619-632, Callis et al. (1990) J Biol Chem 265:12486-12493, Norris et al. (1993) Plant Mol Biol 21:895-906, European Patent Application Nos. 0 342 926 and 0 392 225, Taylor et al (1993) Plant Cell Rep 12:491-495, McElroy et al (1990) Plant Cell 2:163-171, McEclroy et al. (1991) Mol Gen Genet 231:150-160, Chibbar et al. (1993) Plant Cell Rep 12:506-509. Representative inducible promoters suitable for use with the present invention include the chemically inducible PR-1 promoter, the PR-1a promoter, an ethanol-inducible promoter, a glucocorticoid inducible promoter, and a wound-inducible promoter. See Uknes et al. (1992) Plant Cell 4:645-656, Lebel et al. (1998) Plant J 16:223-233, Caddik et al. (1998) Nat Biotechnol 16:177-180, Aoyama & Chua (1997) The Plant Journal 11:605-612, Xu et al. (1993) Plant Mol Biol 22:573-588; Logemann et al. (1989) Plant Cell 1:151-158, Rohrmeier & Lehle (1993) Plant Mol Biol 22:783-792, Firek et al. (1993) Plant Mol Biol 22:129-142, and Warner et al. (1993) Plant J 3:191-201. Selected promoters can direct expression in specific cell types (such as leaf epidermal cells, mesophyll cells, root cortex cells) or in specific tissues or organs (roots, leaves or flowers, for example). Representative promoters that direct cell- or tissue-specific expression in plants and can be used in accordance with the present invention include but are not limited to a root-specific promoter (de Framond (1991) FEBS 290:103-106, U.S. Pat. No. 5,466,785), a pith-preferred promoter (International Publication No. WO 93/07278), a leaf-specific promoter (Hudspeth & Grula (1989) Plant Mol Biol 12:579-589), and a pollen-specific promoter (International Publication No. WO 93/07278).

XI.B. Vectors

The expression cassette is cloned into a vector suitable for transformation. Suitable expression vectors which can be used include, but are not limited to, the following vectors or their derivatives: plant transformation vectors, viruses such as vaccinia virus or adenovirus, baculovirus vectors, yeast vectors, bacteriophage vectors (e.g., lambda phage), plasmid and cosmid DNA vectors, and transposon-mediated transformation vectors.

Numerous vectors available for plant transformation are known to those of ordinary skill in the plant transformation arts, and the genes pertinent to this invention can be used with any such vectors. Exemplary vectors include pCIB200, pCIB2001, pCIB10, pCIB3064, pSOG19, and pSOG35. The selection of vector will depend upon the preferred transformation technique and the target species for transformation.

Many vectors are available for transformation using Agrobacterium tumefaciens. These typically carry at least one T-DNA border sequence and include vectors such as pBIN19 (Bevan (1984) Nuc Acids Res 12:8711-8721) and pXYZ. See also European Patent Application No. 0 332 104, herein incorporated by reference.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g., electroporation), and microinjection. The choice of vector depends largely on the preferred selection for the plant species being transformed.

For certain target species, different antibiotic or herbicide selection markers can be preferred. Selection markers used routinely in transformation include the nptII gene, which confers resistance to kanamycin and related antibiotics (Messing & Vierra (1982) Gene 19: 259-268; Bevan et al. (1983) Nature 304:184-187), the bar gene, which confers resistance to the herbicide phosphinothricin (White et al. (1990) Nuc Acids Res 18:1062, Spencer et al. (1990) Theor Appl Genet 79:625-631), the hph gene, which confers resistance to the antibiotic hygromycin (Blochlinger & Diggelmann (1984) Mol Cell Biol 4:2929-2931), and the dhfr gene, which confers resistance to methatrexate (Bourouis et al. (1983) EMBO J. 2(7): 1099-1104), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

XI.C. Transformation of Dicotyledons

Transformation techniques for dicotyledons are known in the art and include Agrobacterium-based techniques and techniques that do not require Agrobacterium. Non-Agrobacterium techniques involve the uptake of exogenous genetic material directly by protoplasts or cells. This can be accomplished by polyethylene glycol (PEG) electroporation, particle bombardment-mediated uptake, or microinjection. Examples of these techniques are described by Paszkowski et al. (1984) EMBO J. 3:2717-2722; Potrykus et al. (1985) Mol Gen Genet 199:169-177; Reich et al. (1986) Biotechnology 4:1001-1004; Klein et al. (1987) Nature 327:70-73; and U.S. Pat. Nos. 4,945,050, 5,036,006, and 5,100,792. Using any of the aforementioned methods, the transformed cells can be regenerated to whole plants using standard techniques known in the art.

XI.D. Transformation of Monocotyledons

Transformation of most monocotyledon species has now also become routine. Preferred techniques include direct gene transfer into protoplasts using PEG or electroporation techniques, and particle bombardment into callus tissue. See European Patent Application Nos. 0 292 435, 0 392 225, and 0 332 581; International Publication Nos. WO 93/07278 and WO 93/21335; Gordon-Kamm et al. (1990) Plant Cell 2:603-618; Fromm et al. (1990) Biotechnology 8:833-839; Koziel et al. (1993) Biotechnology 11:194-200; Zhang et al. (1988) Plant Cell Rep 7:379-384; Shimamoto et al. (1989) Nature 338:274-277; Datta et al. (1990) Biotechnology 8:736-740; Christou et al. (1991) Biotechnology 9:957-962; Vasil et al. (1992) Biotechnology 10:667-674; Vasil et al. (1993) Biotechnology 11:1553-1558; and Weeks et al. (1993) Plant Physiol 102:1077-1084. More recently, transformation of monocotyledons using Agrobacterium has been described. See International Publication No. WO 94/00977 and U.S. Pat. No. 5,591,616, both of which are incorporated herein by reference.

XII. Methods of Inducible Gene Expression

The present invention further provides a method of controlling gene expression in an organism, the method comprising: (a) transforming the organism with a receptor expression cassette comprising a 5′ regulatory region capable of promoting expression operatively linked to a receptor cassette encoding a chimeric receptor polypeptide of the invention, and a 3′ terminating region; (b) transforming the organism with a target expression cassette comprising a 5′ regulatory region operatively linked to a target nucleotide sequence, wherein the 5′ regulatory region comprises one or more response elements that are recognized by the DNA binding domain of the chimeric receptor polypeptide; (c) expressing the chimeric receptor polypeptide in the organism; and (d) contacting the organism with a chemical ligand that binds to the ligand binding domain of the chimeric receptor polypeptide, whereby the chimeric receptor polypeptide activates expression of the target nucleotide sequence.

Methods employing a gene switch system as disclosed herein are useful for regulated expression in any organism that can express a nuclear receptor expression cassette, including both plants and animals.

In a preferred embodiment, nuclear receptor cassettes comprising disclosed nuclear receptor sequences are useful for the regulation of expression of target polypeptides in plants in the presence of appropriate chemical ligands. For example, U.S. Pat. No. 5,880,333 is drawn to a method for controlling gene expression in plants comprising transforming a plant with expression cassette encoding a nuclear receptor polypeptide and a target sequence. The method is useful for controlling various traits of agronomic importance.

The gene switch system disclosed herein can also be adopted to gene therapy methods. The insect nuclear receptor components of a receptor cassette disclosed herein are particularly relevant to regulated expression in mammals based on their heterologous derivation. Utilization of insect nuclear receptor ligand binding domains, and ligands that specifically activate such nuclear receptors, will minimize the possibility of cross-reactivity with endogenous mammalian receptors.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Example 1

### Database Searches

A pileup alignment of Drosophila and C. elegans nuclear receptor DNA-binding domains was generated using the GCG program (Devereux et al. (1984) Nuc Acids Res 12: 387-395). The pileup was used to build a profile Hidden Markov Model (HMM) according to the HMMER 2.1.1 program (available from Washington University School of Medicine, St. Louis, Mo.). HMMER 2.1.1 hmmbuild parameters were selected for maximal sensitivity for identifying complete fragments and excluding local alignments. The profile HMM was further calibrated according to the program instructions. A database (referred to herein as “the GeneMark database”) was assembled by predicting proteins using the GeneMark program (Borodovsky & McIninch (1993) Computers & Chemistry 17:123-133) based on 50 kb Drosophila genomic sequence scaffolds that had been generated at Celera Genomics, Inc. (Rockville, Md.). The profile HMM was also used to search a database generated by Celera using alternative protein prediction programs (referred to herein as the “Celera predicted protein database”).

### Example 2

### Isolation of Drosophila melanogaster Nuclear Receptor cDNAs

cDNA clones of Drosophila nuclear receptors were cloned by PCR using a first strand Drosophila cDNA pool as template. PCR primers were designed to include the predicted start and stop codons of each receptor using the primer3 application (available through Whitehead/MIT Center for Genome Research of Cambridge, Mass.). Amplified products were cloned in the pUNI/V5-His-TOPO vector (Invitrogen Corporation of Carlsbad, Calif.). Cloned inserts were sequences on both strands by primer walking using an ABI PRISM® 3700 DNA Analyzer (Applied Biosystems of Foster City, Calif.) to an accuracy of <1/10,000 nucleotide errors.

### Example 3

### Cloning Heliothis virescens Nuclear Receptors by Library Screening

A cDNA library was constructed in the Uni-ZAP XR vector Stratagene of La Jolla, Calif.) using oligo-dT-primed transcripts from Heliothis virescens embryos aged 0-24 hours at 25° C. Library clones were excised from Uni-ZAP XR as pBluescript phagemids. The library was transformed into E. coli by electroporation and plated into 384-well plates with an average calculated density of 4-6 colonies per well. Plates were arrayed by a Q-Bot (Genetix Pharmaceuticals, Inc. of Cambridge, Mass.) onto a 22×22 cm nylon filter. The filter was probed under low stringency conditions. Representative low-stringency conditions are overnight hybridization at 55° C. in Church's Buffer followed by washing in 2×SSC 0.1% SDS, twice at room temperature and once at 42° C., each wash approximately 30 min). The probe was a mixture of random primed radioactively labeled DNA binding domains (DBDs) of Drosophila melanogaster nuclear receptors EcR, usp, DHR38, DHR39, βFTZ-F1 and E75. The DBD fragments were generated by PCR using nondegenerate primers corresponding to the indicated DBDs.

For secondary screening, E. coli from positive wells were spread onto agar plates, 96 colonies corresponding to each primary screen well were picked into 384-well plates. The colonies were arrayed onto a nylon membrane and re-probed using identical conditions. Positive clones were sequenced.

### Example 4

### Cloning Heliothis virescens Nuclear Receptors by PCR

polyA⁺ RNA was made from Heliothis virescens embryos aged 0-24 hours at 25° C. cDNA was prepared by reverse transcription using random primers and a Gene Amp kit (Perkin Elmer of San Jose, Calif.). The reverse transcription reaction was allowed to proceed for 15 min at 42° C., and the reaction was stopped by incubating the reaction for 5 min at 99° C. For amplification of nuclear receptors, nested, degenerate primers designed according to the Drosophila βFTZ-F1 sequence (SEQ ID NOs:35-38). Primers included restriction enzyme sites to facilitate cloning into pBluescript (Stratagene of La Jolla, Calif.). Cycling parameters for amplification using degenerate primers were as follows: initial amplification—2 min at 95° C.; 35 cycles—15 sec at 95° C., 30 sec at 60° C.; 7 min at 72° C.; hold at 4° C.; second amplification—2 min at 95° C.; 35 cycles—15 sec at 95° C., 30 sec at 60° C., 2.5 min at 72° C.; 7 min at 72° C.; hold at 4° C.

Amplification of cDNA ends was performed using a MARATHON RACE kit (Clontech Laboratories, Inc. of Palo Alto, Calif.). Gene-specific primers (SEQ ID NOs:39-42) were designed according to Heliothis virescens nuclear receptor sequences obtained as described herein above. cDNA libraries generated from Heliothis virescens adult head and/or larval gut were used as template. RACE products were cloned into the TOPO-A vector (Invitrogen Corporation of Carlsbad, Calif.).

### Example 5

### Double-Stranded RNA Interference

Preparation of dsRNA for Injection. Sequences to be expressed as dsRNA were cloned into Bluescript KS(+) (Stratagene of La Jolla, Calif.), linearized with the appropriate restriction enzymes, and transcribed in vitro with the Ambion T3 and T7 MEGASCRIPT® high yield transcription kits following the manufacturer's instructions (Ambion Inc. of Austin, Tex.). Transcripts were annealed in injection buffer (0.1 mM NaPO₄ pH 7.8, 5 mM KCl) after heating to 85° C. and cooling to room temperature over a 1- to 24-hr period. All annealed transcripts were analyzed on agarose gels with DNA markers to confirm the size of the annealed RNA and quantitated as described previously (Fire et al. (1998) Nature 391(6669):806-811). Injected RNA was not gel-purified. Injection of 0.1 nl of a 0.1- to 1.0-mg/ml solution of a 1-kb dsRNA corresponds to roughly 10⁷ molecules/injection.

Injection of Drosophila melanogaster Embryos. Fly cages were set up using 2- to 4-day flies. Agar-grape juice plates were replaced every hour to synchronize the egg collection for 1-2 days. The eggs were collected over a 30- to 60-min period for subsequent injection. The eggs were washed into a nylon mesh basket with tap water. The chorion was removed by brief soaking in a dilute bleach solution. Eggs were positioned on a glass slide such that each egg was in a same orientation. Double-stranded RNA was injected into middle of each egg using an Eppendorf transjector (Eppendorf Scientific, Inc. of Westbury, N.Y.). Following injection, slides were stored in a moist chamber to prevent dessication of the embryos. Embryos were monitored for development and transferred as first instar larvae to vials containing Drosophila medium. Methods for rearing Drosophila staging and common genetic techniques can be found, for example, in Roberts (1986) Drosophila melanogaster, A Practical Approach, IRL Press, Washington, D.C.; Ashburner (1989a) Drosophila: A Laboratory Handbook, Cold Spring Harbor Laboratory Press, New York, N.Y.; Ashburner (1989b) Drosophila: A Laboratory Manual, Cold Spring Harbor Laboratory Press, New York, N.Y.; Goldstein & Fyrberg, eds (1994) in Methods in Cell Biology, Vol. 44, Academic Press, San Diego, Calif.

### Example 6

### Overexpression of Nuclear Receptors in Drosophila melanogaster

The following Drosophila melanogaster lines were used for overexpression analysis:
w¹¹¹⁸
w¹¹¹⁸; +/SM5; P[hs-E75A w⁺]/TM3
w¹¹¹⁸; P[hs-E75A-H w⁺]/CyO; Dr/TM3
w¹¹¹⁸; P[hs-DHR39-6 w⁺]
w¹¹¹⁸; P[hs-DHR39-3 w⁺]
yw, P[hs-DHR38 w⁺]-II

All lines were acquired from the public Drosophila melanogaster Stock Center of Bloomington, Indiana (http://flystocks.bio.indiana.edu) or from Dr. Carl Thummel of Howard Hughes Medical Institute, Salt Lake City, Utah. Drosophila melanogaster genotypes are indicated according to standard nomenclature in the field. See Lindsley & Zimm (1992) The Genome of Drosophila melanogaster, Academic Press, San Diego, Calif.

Embryos were collected on grape juice/agar plates at 25° C. First instar larvae were staged at embryo hatching. Collections of first instar larvae aged 0-2 hours +/−15 minutes post-hatching were allowed to develop to the desired stage at 25° C.

Heat treatments were performed by placing staged larvae on grape juice/agar plates in a 37° C. warm room for 2 hours. Larvae were then transferred to vials containing Drosophila medium and returned to 25° C. for post-heat treatment recovery. The duration of heat treatment was determined empirically to result in minimal lethality of control lines. Viability was scored at 24 hours post heat treatment by observation of larval movement and/or at four days post heat treatment by counting the number of pupae. All heat treatment experiments were performed using collections of 50 larvae per genotype.

### Example 7

### Recombinant Production of in E. coli

A cDNA clone of the present invention is subcloned into an appropriate expression vector and transformed into E. coli using the manufacturers conditions. Specific examples include plasmids such as pBluescript (Stratagene of La Jolla, Calif.), pFLAG (International Biotechnologies, Inc. of New Haven, Conn.), and pTrcHis (Invitrogen Corporation of Carlsbad, Calif.). E. coli are cultured, expression of the recombinant protein is confirmed, and recombinant protein is isolated using standard techniques.

### Example 8

### Recombinant Production of a Nuclear Receptor in Insect Cells

Baculovirus vectors, which are derived from the genome of AcNPV virus, are designed to provide high levels of expression of cDNA in the Spodoptera frugiperda (SF9) line of insect cells (ATCC CRL# 1711). Recombinant baculovirus expressing the cDNA of the present invention is produced by the following standard methods (Invitrogen MaxBac Manual, Invitrogen Corporation of Carlsbad, Calif.): cDNA constructs are ligated into the polyhedrin gene in any one of a variety of baculovirus transfer vectors, including the pAC360 and the BleBAc vector (Invitrogen Corporation of Carlsbad, Calif.). Recombinant baculoviruses are generated by homologous recombination following co-transfection of the baculovirus transfer vector and linearized AcNPV genomic DNA (Kitts (1990) Nucleic Acid Res 18:5667) into SF9 cells. Recombinant pAC360 viruses are identified by the absence of inclusion bodies in infected cells and recombinant pBlueBac viruses are identified on the basis of β-galactosidase expression (Summers & Smith, Texas Agriculture Exp Station Bulletin No. 1555).

A cDNA encoding an entire open reading frame for gene is inserted into the BamH I site of pBlueBacII (Invitrogen Corporation of Carlsbad, Calif.). Constructs in the positive orientation, identified by sequence analysis, are used to transfect SF9 cells in the presence of linear AcNPV wild type DNA. The recombinant insect nuclear receptor is present in the cytoplasm of infected cells. The recombinant insect nuclear receptor is extracted from infected cells by hypotonic or detergent lysis.

### Example 9

### In Vitro Binding Assays

Recombinant protein can be obtained, for example, according to the approach described in Example 7 or 8 herein above. The protein is immobilized on chips appropriate for ligand binding assays. The protein immobilized on the chip is exposed to a candidate substance according to methods known in the art. While the sample compound is in contact with the immobilized protein, measurements capable of detecting protein-ligand interactions are conducted. Measurement techniques include, but are not limited to, SEDLI, Biacore, and FCS, as described above. Substances that bind the protein are readily discovered using this approach and are subjected to further characterization.

### REFERENCES

The references listed below as well as all references cited in the specification are incorporated herein by reference to the extent that they supplement, explain, provide a background for or teach methodology, techniques and/or compositions employed herein.
Adelman et al. (1983) DNA 2:183.
Alam and Cook (1990) Anal Biochem 188:245-254.
Altschul et al. (1990) J Mol Biol 215: 403-410.
Aoyama & Chua (1997) The Plant Journal 11:605-612.
Ashburner (1989a) Drosophila: A Laboratory Handbook, Cold Spring Harbor Laboratory Press, New York, N.Y.
Ashburner (1989b) Drosophila: A Laboratory Manual, Cold Spring Harbor Laboratory Press, New York, N.Y.
Ausubel et al. (1992) Current Protocols in Molecular Biology, John Wylie and Sons, Inc., New York, N.Y.
Barton (1998) Acta Crystallogr D Biol Crystallogr 54:1139-1146.
Bass (2000) Cell 101(3):235-238.
Batzer et al. (1991) Nuc Acids Res 19:5081.
Bevan (1984) Nuc Acids Res 12:8711-8721.
Bevan et al. (1983) Nature 304:184-187.
Binet et al. (1991) Plant Sci 79:87-94.
Blochlinger & Diggelmann (1984) Mol Cell Biol 4:2929-2931.
Bodanszky, et al. (1976) Peptide Synthesis, John Wiley and Sons, Second Edition, New York, N.Y.
Bonnelye et al. (1997) Mol Endocrinol 11(7):905-916.
Borodovsky & McIninch (1993) Computers & Chemistry 17:123-133.
Bosher & Labouesse (2000) Nat Cell Biol 2(2):E31-36.
Bourguet et al. (1995) Nature 375:377-382.
Bourouis et al. (1983) EMBO J. 2(7):1099-1104.
Brent & Ptashne (1985) Cell 43: 729-736.
Brookes (1999) Gene 234(2):177-186.
Caddik et al. (1998) Nat Biotechnol 16:177-180.
Callis et al. (1990) J Biol Chem 265:12486-12493.
Campos-Ortega & Hartenstein (1985) The Embryonic Development of Drosophila melanogaster, Springer-Verlag, Berlin.
Carney et al. (1997) Proc Natl Acad Sci USA 94(22):12024-12029.
Casida & Quistad (1995) in Casida & Quistad, eds, Pyrethrum Flowers: Production, Chemistry, Toxicology, and Uses, pp. 258-276, Oxford University Press, New York, N.Y.
Chibbar et al. (1993) Plant Cell Rep 12:506-509.
Christensen et al. (1989) Plant Mol Biol 12:619-632.
Christou et al. (1991) Biotechnology 9:957-962.
Coffer et al. (1996) J Steroid Biochem Mol Biol 58:467-477.
Conner et al. (1983) Proc Natl Aced Sci USA 80:278.
Cooper et al. (1996) J Steroid Biochem Mol Biol 57:251-257.
Cubitt et al. (1995) Trends Biochem Sci 20:448-455.
Dallery et al. (1993) Biochem 32:12428-12436.
Danielsen et al. (1989) Cell 57:1131-1138.
Datta et al. (1990) Biotechnology 8:736-740.
de Framond (1991) FEBS 290:103-106.
Demerec (1965) Biology of Drosophila, Hafner Publishing, New York, N.Y.
Desjarlais & Berg (1993) Proc Natl Acad Sci USA 90:2252-1860.
Devereux et al. (1984) Nuc Acids Res 12:387-395.
Dhadialla et al. (1998) Annu Rev Entom 43:545-569.
Dittrich et al. (1997) Development 124(13):2515-2525.
Edwards & Kiehart (1996) Development 122:1499.
Eul et al. (1989) EMBO J. 8:83-90.
European Patent Application No. 0 292 435
European Patent Application No. 0 332 104
European Patent Application No. 0 332 581
European Patent Application No. 0 342 926
European Patent Application No. 0 392 225
Evans (1988) Science 240:889-895.
Fire et al. (1998) Nature 391 (6669):806-811.
Firek et al. (1993) Plant Mol Biol 22:129-142.
Forman & Samuels (1990) Mol Endocrinol 4:1293-1301.
Forman et al. (1989) Mol Endocrinol 3:1610-1626.
Freeman et al. (1999) Development 126:4591-4602.
Fromm et al. (1990) Biotechnology 8:833-839/
Giguere et al. (1987) Nature 330:624-629.
Glover, ed. (1985) DNA Cloning: A Practical Approach, MRL Press, Ltd., Oxford, United Kingdom.
Goff et al. (1991) Genes and Dev 5:298-309.
Goldstein & Fyrberg, eds (1994) in Methods in Cell Biology, Vol. 44, Academic Press, San Diego, Calif.
Gordon-Kamm et al. (1990) Plant Cell 2:603-618.
Green & Chambon (1987) Nature 325:75-78.
Green et al. (1988) EMBO J. 7:3037-3044.
Guichet et al. (1997) Nature 385:548-552.
Hadzic et al. (1995) Mol Cell Biol 15:4507-4517.
Hall et al. (2000) Mol Enodcrinol 14(12):2010-2023.
Hard et al. (1990) Science 249:397-404.
Harding & Lazar (1995) Mol Cell Biol 15(11):6479.
Harlow & Lane (1988) Antibodies: A Laboratory Manual, Cold Spring Harbor Laboratory Press, Cold Spring Harbor, N.Y.
Henikoff & Henikoff (1989) Proc Natl Acad Sci USA 89:10915.
Henikoff & Henikoff (2000) Adv Protein Chem 54:73-97.
Henikoff et al. (2000) Electrophoresis 21(9):1700-1706.
Henrich et al. (1987) Dev Biol 120(1):50-55.
Henrich et al. (1993) Dev Genet 14(5):469-477.
Higashijima et al. (1996) Development 122(2):527-536.
Hodgson & Tate (1976) in Wilkinson, ed, Insecticide Biochemistry and Physiology, pp. 115-148, Plenum Press, New York, N.Y.
Huang et al. (2000) Pac Symp Biocomput 230-241.
Hudspeth & Grula (1989) Plant Mol Biol 12:579-589.
Hunter (2000) Curr Biol 10(4):R137-140.
Hutchens & Yip (1993) Rapid Commun Mass Spectrom 7:576-580.
International Publication No. WO 00/37077
International Publication No. WO 93/07278
International Publication No. WO 93/21335
International Publication No. WO 94/00977
International Publication No. WO 97/47763
International Publication No. WO 99/26966
Johnson & Garza (1998) Ann Dros Res Conf 39:430A.
Johnston et al. (1997) Mol Endocrinol 11(3):342-352.
Karlin & Altschul (1993) Proc Natl Acad Sci USA 90:5873-5887.
Kennerdell & Carthew (2000) Nat Biotech 18(8):896-898.
Kestila et al. (1998) Mol Cell 1(4):575-582.
Kitts (1990) Nucleic Acid Res 18:5667.
Klein et al. (1987) Nature 327:70-73.
Koelle (1992) Ph.D. Thesis, Stanford University, Stanford, Calif.
Koelle et al. (1991) Cell 67:59-77.
Koziel et al. (1993) Biotechnology 11:194-200.
Kuwabara & Olson (2000) Parasitol Today 16(8):347-349.
Kyte et al. (1982) J Mol Biol 157:105.
Lam & Thummel (2000) Curr Biol 10(16):957-963.
Lam et al. (1999) Dev Biol 212(1):204-216.
Landegren et al. (1998) Genome Res 8:769-776.
Landgren et al. (1988) Science 241:1007.
Landgren et al. (1988) Science 242:229-237.
Lanz & Rusconi (1994) Endocrinology 135:2183-2194.
Laudet et al. (1992) EMBO J. 11:1003-1013.
Lazar & Harding (1998) in Freedman, ed, Molecular Biology of Steroid and Nuclear Hormone Receptors, pp. 269-270, Birkhäuser, Boston, Mass.
Lebel et al. (1998) Plant J 16:223-233.
Leng et al. (1995) Mol Cell Biol 15:255-263.
Liedberg et al. (1983) Sensors Actuators 4:299-304.
Lin et al. (1991) Mol Endocrinol 5:485-492.
Lindsley & Zimm (1992) The Genome of Drosophila melanogaster, Academic Press, San Diego, Calif.
Lis et al. (1983) Cell 35:403.
Liu et al. (1997) Proc Natl Acad Sci USA 94:5525-5530.
Logemann et al. (1989) Plant Cell 1:151-158.
Lowman (1997) Annu Rev Biophys Biomol Struct 26:401-424.
Lucknow & Summers (1988) Bio/Technol 6:47.
Lupisella et al. (1995) J Biol Chem 270:24884-24890.
Maalouf et al. (1998) J Biomol Struc Dynamics 15(5):841-851.
Mader et al. (1993) EMBO J. 12:5029-5041.
Madge et al. (1972) Phys Rev Lett 29:705-708.
Maiti et al. (1997) Proc Natl Aced Sci USA 94:11753-11757.
Mak et al. (1989) J Biol Chem 264:21613:21618.
Malmquist (1993) Nature 361:186-187.
McEclroy et al. (1991) Mol Gen Genet 231:150-160.
McElroy et al (1990) Plant Cell 2:163-171.
Meijer (2000) Mol Gen Genet 263:12-21.
Meshi (1995) Plant Cell Physiol 36:1405-1420.
Messing & Vierra (1982) Gene 19:259-268.
Misquitta & Paterson (1999) Proc Natl Acad Sci USA 96 (4):1451-1456.
Nauber et al. (1988) Nature 336(6198):489-492.
Needleman & Wunsch (1970) J Mol Biol 48:443.
Nemoto et al. (1992) J Steroid Biochem Mol Biol 42:803-812.
Ng (2000) Trends Biochem Sci 25:121-126.
Nijhout (1994) Insect Hormones, Princeton University Press, Princeton, N.J.
Norris et al. (1993) Plant Mol Biol 21:895-906.
Northrop et al. (2000) Mol Endocrinol 14(5)605-622.
Ohtsuka et al. (1985) J Biol Chem 260:2605-2608.
Orita et al. (1989) Proc Natl Aced Sci USA 86(8):2766-2770.
Oro et al. (1988) Nature 336(61989):493-496.
Oro et al. (1992) Development 115(2): 449-462.
Pakdel et al. (1993) Mol Endocrinol 7:1408-1417.
Paszkowski et al. (1984) EMBO J. 3:2717-2722.
Pearson & Lipman (1988) Proc Natl Acad Sci USA 85:2444-2448.
Perlmann et al. (1993) Genes Dev 7:1411-1422.
Perrimon et al. (1985) Genetics 11:23-41.
Perry et al. (1997) Insecticides in Agriculture and Environment: Retrospects and Prospects, pp. 7-10, Springer-Verlag, New York, N.Y.
Perry et al. (1997) Insecticides in Agriculture and Environment: Retrospects and Prospects, pp. 12-13, Springer-Verlag, New York, N.Y.
Potrykus et al. (1985) Mol Gen Genet 199:169-177.
Ptashne (1988) Nature 335:683-689.
Rastinejad (1998) in Freedman, ed, Molecular Biology of Steroid and Nuclear Hormone Receptors, pp. 107, Birkhäuser, Boston, Mass.
Rastinejad (1998) in Freedman, ed, Molecular Biology of Steroid and Nuclear Hormone Receptors, pp. 103-131, Birkhäuser, Boston, Mass.
Rastinejad et al. (1995) Nature 375:203-211.
Reich et al. (1986) Biotechnology 4:1001-1004.
Roberts (1986) Drosophila melanogaster, A Practical Approach, IRL Press, Washington, D.C.
Rochel et al. (1997) Biochem Biophys Res Commun 230:293-296.
Rohrmeier & Lehle (1993) Plant Mol Biol 22:783-792.
Rose and Botstein (1983) Meth Enzymol 101:167-180.
Rossolini et al. (1994) Mol Cell Probes 8:91-98.
Rusconi & Yamamoto (1987) EMBO J. 6:1309-1315.
Sadowski et al. (1988) Nature 335:563-564.
Saiki et al. (1985) Bio/Technology 3:1008-1012.
Sambrook et al., eds (1989) Molecular Cloning: A Laboratory Manual, Cold Spring Harbor Laboratory Press, Cold Spring Harbor, N.Y.
Saqi et al. (1999) Bioinformatics 15(6):521-522.
Schapira et al. (2000) Proc Natl Acad Sci USA 97(3):1008-1013.
Schneuwly et al. (1987) Nature 325:816.
Segraves & Hogness (1990) Genes Dev 4:204-219.
Sharp (1999) Genes Dev 13(2):139-141.
Shimamoto et al. (1989) Nature 338:274-277.
Sidhu (2000) Curr Opin Biotech 11(6):610-616.
Silhavy et al. (1984) Experiments with Gene Fusions, Cold Spring Harbor Laboratory Press, Cold Spring Harbor, N.Y.
Simental et al. (1991) J Biol Chem 266:510-518.
Simons in Freedman, ed, Molecular Biology of Steroid and Nuclear Hormone Receptors, pp. 35-104, Birkhäuser, Boston, Mass.
Sliter & Gilbert (1992) Genetics 130:555-568.
Smith & Waterman (1981) Adv Appl Math 2:482.
Spencer et al. (1990) Theor Appl Genet 79:625-631.
Stoneking et al. (1991) Am J Hum Genet 48(2):370-82.
Struhl (1985) Nature 318:677.
Summers & Smith Texas Agriculture Exp Station Bulletin No. 1555.
Sutherland et al. (1995) Proc Natl Acad Sci USA 92(17):7966-7970.
Taylor et al (1993) Plant Cell Rep 12:491-495.
Tetel et al. (1997) Mol Endocrinol 11:1114-1128.
Thomas et al. (1993) Nature 362:471-475.
Thompson et al. (1989) Proc Natl Acad Sci USA 86:3493-3498.
Thummel (1995) Cell 83:1-20.
Thummel (1997) BioEssays 19(8):669-672.
Tijssen (1993) Laboratory Techniques in Biochemistry and Molecular Biology-Hybridization with Nucleic Acid Probes, part I chapter 2, Elsevier, New York, N.Y.
Triezenberg et al. (1988) Genes Dev 2(6): 718-729.
Tsai et al. (1998) in Wilson et al., eds, Williams Textbook of Endocrinology, pp. 55-94, W.B. Saunders Company, Philadelphia, Pa.
U.S. Pat. No. 4,196,265
U.S. Pat. No. 4,554,101
U.S. Pat. No. 4,940,935
U.S. Pat. No. 4,945,050
U.S. Pat. No. 4,981,784
U.S. Pat. No. 5,036,006
U.S. Pat. No. 5,100,792
U.S. Pat. No. 5,188,642
U.S. Pat. No. 5,260,203.
U.S. Pat. No. 5,466,785
U.S. Pat. No. 5,510,240
U.S. Pat. No. 5,591,616
U.S. Pat. No. 5,686,600
U.S. Pat. No. 5,767,378
U.S. Pat. No. 5,834,228
U.S. Pat. No. 5,872,011
U.S. Pat. No. 5,880,333
U.S. Pat. No. 5,994,629
Ueda et al. (1992) Mol Cell Biol 12(12):5667-5672.
Uknes et al. (1992) Plant Cell 4:645-656.
Umesono & Evans (1989) Cell 57:1139-1146.
Vasil et al. (1992) Biotechnology 10:667-674.
Vasil et al. (1993) Biotechnology 11:1553-1558.
Wagner et al. (1995) Nature 378:690-697.
Wang et al. (1998) Science 280(5366): 1077-1082.
Warner et al. (1993) Plant J 3:191-201.
Weeks et al. (1993) Plant Physiol 102:1077-1084
Wentworth et al. (2000) J Endocrinol 166(3):R11-16.
White et al. (1990) Nuc Acids Res 18:1062.
White et al. (1997) Science 276(5309):114-117.
Wilkinson (1976a) in Tahori, ed, Proc 2^{nd} nt Congr on Pesticides and Chemistry, Vol. 2, pp. 117-159, Gordon & Breach, New York, N.Y.
Wilkinson (1976b) in Metcalf & McKelvey, eds, The Future for Insecticides: Needs and Prospects, Vol. 6, pp. 191-178, Wiley, New York, N.Y.
Wilson et al. (1992) Science 256:106-110.
Wilson et al. (1993) Mol Cell Biol 13(9):5794-5804.
Wing (1988) Science 241:467-469.
Woodard et al. (1994) Cell 79(4):607-615.
Worrall et al. (1998) Anal Biochem 70:750-756.
Wu (2000) Plant J 22:19-27.
Wurtz et al. (1996) Nat Struct Biol 3:87-94.
Xu et al. (1993) Plant Mol Biol 22:573-588.
Xu et al. (1996) J Biol Chem 271:21430-21438.
Yamamoto (1973) in Casida, ed, Pyrethrum, The Natural Insecticide, pp. 191-170, Academic Press, New York, N.Y.
Yang & Chen (1999) Cancer Res 59(18):4519-4524.
Yao (1993) Cell 71:63-72.
Yu et al. (1997) Nature 385:552-555.
Yuan et al. (1999) Hum Mutat 14(5):440-446.
Zechel et al. (1994) EMBO J. 13:1414-1424.
Zhang et al. (1988) Plant Cell Rep 7:379-384.
Zhang et al. (1996a) Mol Endocrinol 10:24-34.
Zhang et al. (1996b) J Biol Chem 271:14825-14833.
Zimmer et al. (1993) Peptides, pp. 393-394, ESCOM Science Publishers, B. V.

It will be understood that various details of the invention can be changed without departing from the scope of the invention. Furthermore, the foregoing description is for the purpose of illustration only, and not for the purpose of limitation—the invention being defined by the claims appended hereto.

1. An isolated insect nuclear receptor polypeptide comprising:
(a) a polypeptide encoded by the nucleotide sequence of any one of SEQ ID NOs:1, 5, 9, 17, 19, 21, 23, and 25;
(b) a polypeptide encoded by a nucleic acid molecule that is substantially identical to any one of SEQ ID NOs:1, 5, 9, 17, 19, 21, 23, and 25;
(c) a polypeptide comprising the amino acid sequence of any one of SEQ ID NOs:2, 6, 10, 18, 20, 22, 24, and 26;
(d) a polypeptide that is a biological equivalent of the polypeptide of any one of SEQ ID NOs:2, 6, 10, 18, 20, 22, 24, and 26; or
(e) a polypeptide which is immunologically cross-reactive with an antibody that shows specific binding with a polypeptide of any one of SEQ ID NOs:2, 6, 10, 18, 20, 22, 24, and 26.

2. An isolated nucleic acid molecule encoding an insect nuclear receptor polypeptide comprising:
(a) a nucleotide sequence of any one of SEQ ID NOs:1, 5, 9, 17, 19, 21, 23, and 25; or
(b) a nucleic acid molecule substantially identical to any one of SEQ ID NOs:1, 5, 9, 17, 19, 21, 23, and 25.

3. A chimeric gene, comprising the nucleic acid molecule of claim 2 operatively linked to a heterologous promoter.

4. A vector comprising the chimeric gene of claim 3.

5. A host cell comprising the chimeric gene of claim 3.

6. The host cell of claim 5, wherein the cell is selected from the group consisting of a bacterial cell, an insect cell, and a plant cell.

7. A method of detecting a nucleic acid molecule that encodes an insect nuclear receptor polypeptide, the method comprising:
(a) procuring a biological sample comprising nucleic acid material;
(b) hybridizing the nucleic acid molecule of claim 2 under stringent hybridization conditions to the biological sample of (a), thereby forming a duplex structure between the nucleic acid of claim 2 and a nucleic acid within the biological sample; and
(c) detecting the duplex structure of (b), whereby a nuclear receptor nucleic acid molecule is detected.

8. An antibody that specifically recognizes an insect nuclear receptor polypeptide of claim 1.

9. A method for producing an antibody that specifically recognizes an insect nuclear receptor polypeptide, the method comprising:
(a) recombinantly or synthetically producing an insect nuclear receptor polypeptide, or portion thereof, as set forth in any of SEQ ID NOs:2, 6, 10, 18, 20, 22, 24, and 26;
(b) formulating the polypeptide of (a) whereby it is an effective immunogen;
(c) administering to an animal the formulation of (b) to generate an immune response in the animal comprising production of antibodies, wherein antibodies are present in the blood serum of the animal; and
(d) collecting the blood serum from the animal of (c), the blood serum comprising antibodies that specifically recognize a nuclear receptor polypeptide.

10. A method for detecting a level of nuclear receptor polypeptide, the method comprising
(a) obtaining a biological sample comprising peptidic material; and
(b) detecting a nuclear receptor polypeptide in the biological sample of (a) by immunochemical reaction with the antibody of claim 8, whereby a level of nuclear receptor polypeptide in a sample is determined.

11. A method for identifying a substance that modulates nuclear receptor function, the method comprising:
(a) isolating an insect nuclear receptor polypeptide of claim 1;
(b) exposing the isolated insect nuclear receptor polypeptide to a plurality of candidate substances;
(c) assaying binding of a candidate substance to the isolated nuclear receptor polypeptide; and
(d) selecting a candidate substance that demonstrates specific binding to the isolated insect nuclear receptor polypeptide.

12. A method for identifying an insecticidal substance that modulates nuclear receptor function, the method comprising:
(a) isolating an insect nuclear receptor polypeptide of any one of even numbered SEQ ID NOs:2-34, wherein modulation of the insect nuclear receptor polypeptide confers lethality of an insect during a larval stage;
(b) exposing the isolated insect nuclear receptor polypeptide to a plurality of substances;
(c) assaying binding of a substance to the isolated nuclear receptor polypeptide; and
